(19) United States
(12) Patent Application Publication
(10) Pub. No.: US 2026/0311803 A1
(43) Pub. Date: Oct. 8, 2026

(54) COMPUTATIONAL METHODS FOR SELECTING PERSONALIZED NEOANTIGEN VACCINES

(71) Applicant: Icahn School of Medicine at Mount Sinai, New York, NY (US)

(72) Inventors: Tim O'DONNELL, New York, NY (US); Julia KODYSH, New York, NY (US); Nina BHARDWAJ, New York, NY (US)

(21) Appl. No.: 19/474,041

(22) PCT Filed: Apr. 12, 2024
(86) PCT No.: PCT/US2024/024443

§ 371 Date: Oct. 9, 2025

Related U.S. Application Data
(60) Provisional application No. 63/495,637, filed on Apr. 12, 2023.

(51) Int. Cl. G16B 20/20; A61K 39/00; C12Q 1/6886; G16H 20/10

(52) U.S. Cl. CPC G16B 20/20; A61K 39/0011; C12Q 1/6886; G16H 20/10

(57) ABSTRACT

Systems and methods for identifying a personalized tumor vaccine are provided. Somatic variants for a subject are determined and RNA sequence reads from a tumor of the subject are obtained. From the RNA sequence reads, fusion proteins are determined, each fusion protein a fusion of a portion of a first protein and a portion of a second protein. Candidate neoantigens are selected, including a first subset of candidate neoantigens encoding somatic variants and a second subset of candidate neoantigens encoding residues of the portions of the first and second proteins. A score is determined for each candidate neoantigen using a scoring function including a first scoring term that upweights candidate neoantigens having a higher relative class I MHC affinity, given a class I HLA type of the subject. Two or more candidate neoantigens are selected for the tumor vaccine as a final set of neoantigens based on the respective scores.

### CROSS REFERENCE TO RELATED APPLICATIONS

This application claims priority to U.S. Provisional Patent Application Ser. No. 63/495,637, filed Apr. 12, 2023, which is hereby incorporated by reference in its entirety.

### REFERENCE TO SEQUENCE LISTING SUBMITTED ELECTRONICALLY

This application contains a Sequence Listing in XML format submitted electronically herewith via EFS-Web. The contents of the XML copy, created on Apr. 12, 2024, is named “1045935046WO_SL.xml” and is 22000 bytes in size. The Sequence Listing is incorporated herein by reference in its entirety.

### TECHNICAL FIELD

The present disclosure relates generally to systems and methods for identifying a tumor vaccine personalized to a human subject afflicted with a cancer, where the tumor vaccine includes neoantigens.

### BACKGROUND

Cancer-specific neoantigens, resulting from genetic alterations accumulated by tumor cells, encode novel stretches of amino acids that are not present in the normal genome. These tumor-specific peptides therefore have not been negatively selected by the immune system as “self” proteome. Thus, total neoantigen load inferred through in silico analysis of whole-exome sequencing data from patients' tumors can be used as a predictor of positive responses to immunotherapy regimens as well as used for vaccine design, including but not limited to peptide-based vaccine, RNA vaccine, and/or DNA vaccine design (Luksza, M. et al. Nature 551, 517-520 (2017); Balachandran, V. P. et al. Nature 551, S12-S16 (2017); Charoentong, P. et al. Cell Reports 18, 248-262 (2017); Hugo, W. et al. Cell 165, 35-44 (2016)). Neoantigen vaccines are developed by comparing the genotype of tumor cells with patient's matching normal tissue or blood. Collected somatic missense and frameshift mutations are then converted to corresponding tumor-specific peptides, which then are screened for MHC-I epitopes through running either experimental functional tests or in silico prediction algorithms. Several algorithms exist, optimizing prediction of epitope-HLA interactions in silico, making it possible to predict MHC class I, and to a lesser extent, MHC class II tumor neoepitopes. Alternatively, mass-spectrometry based approaches to predict tumor epitopes now exist as well. Subsequently, these epitopes can be used for short and long peptide-based vaccines, boosting dendritic-cell (DC) based vaccinations, priming adoptive autologous T cell transfer, and gene-modified cell therapies (Branca, M. A. Nat. Biotechnol. 34, 1019-1024 (2016)).

In this regard, multiple research groups have reported encouraging results of neoantigen-based cancer vaccines that generate tumor antigen specific immune responses, both in mouse models and clinical trials. Additionally, both the quantity and quality of neoantigens has been shown to have predictive value for clinical outcomes in checkpoint-blockade immunotherapy in certain tumor types. Neoantigen recognition by vaccination or through adoptive T cell therapy may have unprecedented potential to advance cancer immunotherapy in combination with other approaches (Roudko V. et al. Front. Immunol. 11:27, 1-11 (2020)). Given the above background, what is needed in the art are systems and methods for improved treatment of human cancers. Further, given the above background, there is a need for developing tumor vaccines that utilize tumor-associated antigens or neoantigens for improved immune response.

### SUMMARY

The present disclosure addresses the need in the art for systems and methods for determining the personalized treatment of human cancers. One of the barriers to overcome in cancer treatment is the immunosuppressive tumor microenvironment (see, e.g., Quail D F, Joyce J A. Cancer Cell. 2017; 31(3): 326-341, which is hereby incorporated herein by reference in its entirety). Tumor mutations lead to the formation of tumor-specific antigens, called neoantigens, that are absent from normal tissue, and which can be recognized by the immune system, providing a specific target for anti-tumor treatment. Neoantigen vaccines are a new tool to treat patients with cancer. They can be instrumental in immunomodulation as expected to amplify the endogenous repertoire of tumor-specific T cells to eliminate any remaining tumor cells. Generally, such therapeutic approaches based on the manipulation of the adaptive immune system are likely to have a relatively low risk for toxicity and thus a favorable side-effect profile due to the inherent specificity of the induced immune response; in addition, these immune-based therapeutics can potentially promote lasting disease remissions due to the introduction and persistence of activated immune cell populations in patients following a course of treatment.

Several groups have used therapeutic vaccines targeting neoantigens to clear tumors in murine models. Consequently, many human neoantigen vaccine trials have been initiated and several have published promising early results. However, since very few cancer mutations are recurrent between patients, the identification of neoantigens is improved using a personalized genomic approach (see, e.g., Rubinsteyn A. et al. Front. Immunol. 8:1807, 1-7 (2018), which is hereby incorporated herein by reference in its entirety).

As such, one aspect of the present disclosure provides a method for identifying a tumor vaccine personalized to a human subject afflicted with a cancer. The method includes determining a first plurality of somatic variants of the subject. A first plurality of sequence reads is obtained from RNA molecules in a sample of a tumor obtained from the subject. From the first plurality of sequence reads, one or more fusion proteins encoded by the first plurality of sequence reads are determined, where each respective fusion protein in the one or more fusion proteins is a fusion of a portion of a respective first human protein and a portion of a respective second human protein. A plurality of candidate neoantigens comprising a first subset of candidate neoantigens and a second subset of candidate neoantigens is selected, where each neoantigen in the first subset of candidate neoantigens encodes a somatic variant in the first plurality of somatic variants, and each neoantigen in the second subset of candidate neoantigens encodes one or more residues from the portion of the respective first human protein and one or more residues from the portion of the respective second human protein. A respective score for each respective candidate neoantigen in the plurality of candidate neoantigens is determined using a scoring function that includes a first scoring term that upweights respective candidate neoantigens having a higher class I major histocompatibility complex (MHC) affinity relative to respective candidate neoantigens having a lower class I MHC affinity, given a class I human leukocyte antigen (HLA) type of the human subject. The method further includes selecting, for the tumor vaccine, two or more candidate neoantigens in the plurality of candidate neoantigens as a final set of neoantigens based on the respective score of each candidate neoantigen in the plurality of candidate neoantigens.

Another aspect of the present disclosure provides a tumor vaccine including a plurality of neoantigenic peptides and an adjuvant personalized for a subject afflicted with a cancer, where a first neoantigen in the tumor vaccine encodes a single nucleotide polymorphism present in RNA molecules in a tumor biopsy obtained from the subject, and a second neoantigen in the tumor vaccine encodes one or more residues from a portion of a first human protein and one or more residues from a portion of a second human protein, where the tumor biopsy includes RNA molecules that are a fusion of the first human protein and the second human protein.

Another aspect of the present disclosure includes a method for identifying a tumor vaccine personalized to a human subject afflicted with a cancer, the method including determining a first plurality of somatic variants of the subject and selecting a plurality of candidate neoantigens where each neoantigen in at least a subset of candidate neoantigens in the plurality of candidate neoantigens encodes a somatic variant in the first plurality of somatic variants. The method further includes determining a respective score for each respective candidate neoantigen in the plurality of candidate neoantigens using a scoring function that comprises a first scoring term that upweights candidate neoantigens having a higher class I major histocompatibility complex (MHC) affinity relative to candidate neoantigens having a lower class I MHC affinity, given a class I human leukocyte antigen (HLA) type of the human subject, and a second scoring term that upweights candidate neoantigens having higher hydrophobicity relative to candidate neoantigens having lower hydrophobicity. The method further includes selecting, for the tumor vaccine, two or more candidate neoantigens in the plurality of candidate neoantigens as a final set of neoantigens based on the respective score of each candidate neoantigen in the plurality of candidate neoantigens.

Yet another aspect of the present disclosure includes a method for identifying a tumor vaccine personalized to a human subject afflicted with a cancer. The method includes obtaining a first plurality of sequence reads from RNA molecules in a sample of a tumor obtained from the subject; obtaining a second plurality of sequence reads from DNA molecules in the tumor sample obtained from the subject; obtaining a third plurality of sequence reads from DNA molecules in a normal tissue sample proximate to an original location of the tumor in the subject; and using the second plurality of sequence reads and the third plurality of sequence reads to identify a plurality of somatic variants of the subject by including in the plurality of somatic variants those somatic variants observed in the second plurality of sequence reads that are not observed in the third plurality of sequence reads. The method further includes selecting a plurality of candidate neoantigens, where each neoantigen in at least a subset of candidate neoantigens in the plurality of candidate neoantigens encodes a somatic variant in the plurality of somatic variants; and determining a respective score for each respective candidate neoantigen in the plurality of candidate neoantigens using a scoring function that comprises a first scoring term that upweights candidate neoantigens having a higher class I major histocompatibility complex (MHC) affinity relative to candidate neoantigens having a lower class I MHC affinity, given a class I human leukocyte antigen (HLA) type of the human subject. The method further includes selecting, for the tumor vaccine, two or more candidate neoantigens in the plurality of candidate neoantigens as a final set of neoantigens based on the respective score of each candidate neoantigen in the plurality of candidate neoantigens.

Still another aspect of the present disclosure includes a method for identifying a tumor vaccine personalized to a human subject afflicted with a cancer. The method includes determining a first plurality of somatic variants of the subject; and selecting a plurality of candidate neoantigens wherein each neoantigen in at least a subset of candidate neoantigens in the plurality of candidate neoantigens encodes a somatic variant in the plurality of somatic variants. The method further includes determining a respective score for each respective candidate neoantigen in the plurality of candidate neoantigens using a scoring function that comprises: a first scoring term that upweights respective candidate neoantigens having a higher class I major histocompatibility complex (MHC) affinity relative to respective candidate neoantigens having a lower class I MHC affinity, given a class I human leukocyte antigen (HLA) type of the human subject, and a second scoring term that upweights respective candidate neoantigens having a higher class II MHC affinity relative to respective candidate neoantigens having a lower class II MHC affinity, given a class II HLA type of the human subject. The method further includes selecting, for the tumor vaccine, two or more candidate neoantigens in the plurality of candidate neoantigens as a final set of neoantigens based on the respective score of each candidate neoantigen in the plurality of candidate neoantigens.

Another aspect of the present disclosure includes a system for identifying a tumor vaccine personalized to a human subject afflicted with a cancer, including a memory; one or more processors; and one or more modules stored in the memory and configured for execution by the one or more processors, the one or more modules including instructions for performing any of the methods disclosed above.

Another aspect of the present disclosure includes a non-transitory computer readable storage medium for identifying a tumor vaccine personalized to a human subject afflicted with a cancer, the non-transitory computer readable storage medium storing one or more programs for execution by one or more processors of a computer system, the one or more computer programs including instructions for performing any of the methods disclosed above.

### BRIEF DESCRIPTION OF THE DRAWINGS

FIG. 1 illustrates an exemplary system topology for identifying a tumor vaccine personalized to a human subject afflicted with a cancer, in accordance with an embodiment of the present disclosure.

FIGS. 2A and 2B collectively illustrate a device for identifying a tumor vaccine personalized to a human subject afflicted with a cancer, in accordance with an embodiment of the present disclosure.

FIGS. 3A, 3B, 3C, 3D, 3E, and 3F collectively provide a flow chart of processes and features for identifying a tumor vaccine personalized to a human subject afflicted with a cancer, in which optional steps are indicated by dashed lines, in accordance with some embodiments of the present disclosure.

FIG. 4 illustrates an example platform for prediction of immunogenic neoantigens, in accordance with an embodiment of the present disclosure.

FIG. 5 illustrates an example platform for prediction of immunogenic neoantigens, in accordance with an embodiment of the present disclosure.

FIG. 6 illustrates an example pipeline for personalized genome vaccine preparation and use, in accordance with an embodiment of the present disclosure.

FIG. 7 illustrates example coverage of patients vaccinated with a combination of somatic variant-based neoantigens and fusion protein-based neoantigens, in accordance with an embodiment of the present disclosure.

FIG. 8 illustrates example approaches for detecting fusion proteins in sequence reads from RNA molecules, in accordance with some embodiments of the present disclosure.

FIG. 9 illustrates example fusion proteins detected in tumor samples, in accordance with an embodiment of the present disclosure.

FIG. 10 illustrates an example approach for detecting breakpoints to identify fusion proteins in sequence reads from RNA molecules, in accordance with some embodiments of the present disclosure.

FIG. 11 illustrates example immunogenicity data in which hydrophobic neoantigens are more likely to elicit a T cell response, in accordance with an embodiment of the present disclosure.

FIGS. 12A and 12B collectively illustrate example priming of T cells from healthy donors using predicted fusion sequences for gene fusions in prostate cancer, in accordance with an embodiment of the present disclosure.

Like reference numerals refer to corresponding parts throughout the several views of the drawings.

### DETAILED DESCRIPTION

Tumor progression is typically accompanied by an accumulation of driver and passenger somatic mutations. A handful of those mutations occur in protein coding genes which introduce non-synonymous polymorphisms. Certain substitutions may give rise to novel, tumor-associated antigens or neoantigens, presentable by cancer cells to the host adaptive immune system. As antigen recognition is the core of an effective immune response, the identification of patient tumor specific antigens derived from transformed cells is of importance for immunotherapeutic approaches. Recent technological advances in DNA sequencing of tumor genomes, advances in gene expression analysis, algorithm development for antigen predictions and methods for T cell receptor (TCR) repertoire sequencing have facilitated the selection of candidate immunogenic neoantigens. See, e.g., Roudko V. et al. Front. Immunol. 11:27, 1-11 (2020), which is hereby incorporated herein by reference in its entirety.

Several groups have used therapeutic vaccines targeting neoantigens to clear tumors in murine models. Consequently, many human neoantigen vaccine trials have been initiated and several have published promising early results. Nevertheless, tumor-based heterogeneity is a common cause of cancer therapy failure. A major impediment to the development of effective cancer vaccines has been the lack of truly tumor specific antigens. As indicated, above, there is a clear unmet need for developing targeted treatments based on the genetic signature of a patient's tumor. Since very few cancer mutations are recurrent between patients, the identification of neoantigens is improved using a personalized genomic approach (see, e.g., Rubinsteyn A. et al. Front. Immunol. 8:1807, 1-7 (2018), which is hereby incorporated herein by reference in its entirety).

Advantageously, the presently disclosed subject matter provides strategies for improving cancer therapy (e.g., immunotherapy) by providing systems and methods for identifying a personalized tumor vaccine. Somatic variants for a subject are determined and RNA sequence reads from a tumor of the subject are obtained. From the RNA sequence reads, fusion proteins are determined, each fusion protein a fusion of a portion of a first protein and a portion of a second protein. Candidate neoantigens are selected, including a first subset of candidate neoantigens encoding somatic variants and a second subset of candidate neoantigens encoding residues of the portions of the first and second proteins. A score is determined for each candidate neoantigen using a scoring function including a first scoring term that upweights candidate neoantigens having a higher relative class I MHC affinity, given a class I HLA type of the subject. Two or more candidate neoantigens are selected for the tumor vaccine as a final set of neoantigens based on the respective scores.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### 1. Definitions

Unless defined otherwise, all technical and scientific terms used herein have the meaning commonly understood by a person skilled in the art to which this invention belongs. The following references provide one of ordinary skill in the art with a general definition of many of the terms used herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991); Molecular Cloning: a Laboratory Manual 3rd edition, J. F. Sambrook and D. W. Russell, ed. Cold Spring Harbor Laboratory Press 2001; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions are hereby incorporated by reference as part of the presently disclosed subject matter. As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “neoantigen,” “neoepitope” or “neopeptide” refers to a tumor-specific antigen that arises from one or more tumor-specific mutation, which alters the amino acid sequence of genome encoded proteins.

As used herein, the term “neoantigen number” or “neoantigen burden” refers to the number of neoantigen(s) measured, detected, or predicted in a sample (e.g., a biological sample from a subject). In certain embodiments, the neoantigen number is measured by using whole exome sequencing and in silico prediction.

As used herein, the term “mutation” refers to permanent change in the DNA sequence that makes up a gene. In certain embodiments, mutations range in size from a single DNA building block (DNA base) to a large segment of a chromosome. In certain embodiments, mutations can include missense mutations, frameshift mutations, duplications, insertions, nonsense mutation, deletions, and/or repeat expansions. In some embodiments, mutations include insertion-deletion (indels), single nucleotide variants (SNVs), multi-nucleotide variants (MNVs), copy number variations (CNVs), and/or genomic rearrangements. In certain embodiments, a missense mutation is a change in one DNA base pair that results in the substitution of one amino acid for another in the protein made by a gene. In certain embodiments, a nonsense mutation is also a change in one DNA base pair. Instead of substituting one amino acid for another, however, the altered DNA sequence prematurely signals the cell to stop building a protein. In certain embodiments, an insertion changes the number of DNA bases in a gene by adding a piece of DNA. In certain embodiments, a deletion changes the number of DNA bases by removing a piece of DNA. In certain embodiments, small deletions can remove one or a few base pairs within a gene, while larger deletions can remove an entire gene or several neighboring genes. In certain embodiments, a duplication consists of a piece of DNA that is abnormally copied one or more times. In certain embodiments, frameshift mutations occur when the addition or loss of DNA bases changes a gene's reading frame. A reading frame consists of groups of 3 bases that each code for one amino acid. In certain embodiments, a frameshift mutation shifts the grouping of these bases and changes the code for amino acids. In certain embodiments, insertions, deletions, and duplications can all be frameshift mutations. In certain embodiments, a repeat expansion is another type of mutation. In certain embodiments, nucleotide repeats are short DNA sequences that are repeated a number of times in a row. For example, a trinucleotide repeat is made up of 3-base-pair sequences, and a tetranucleotide repeat is made up of 4-base-pair sequences. In certain embodiments, a repeat expansion is a mutation that increases the number of times that the short DNA sequence is repeated.

As used herein, the term “somatic variant” refers to a variant arising as a result of dysregulated cellular processes associated with neoplastic cells, e.g., a mutation. In some embodiments, somatic variants are detected via subtraction from a matched normal sample. In some embodiments, a somatic variant includes missense mutations, frameshift mutations, duplications, insertions, nonsense mutation, deletions, and/or repeat expansions. In some embodiments, a somatic variant includes insertion-deletion (indels), single nucleotide variants (SNVs), multi-nucleotide variants (MNVs), copy number variations (CNVs), genomic rearrangements, fusions and/or splice variants. As used herein, the term “splice variant” refers to one or more RNA variants (e.g., isoforms) generated from the transcript of a same gene through different combinations of exons and introns (e.g., alternative splicing). As used herein, the term “fusion” refers to a hybrid polymer (e.g., a fusion gene and/or a fusion protein) formed from two previously separate polymers (e.g., genes and/or proteins). In some embodiments, fusions occur as a result of one or more of translocation, interstitial deletion, and/or chromosomal inversion.

As used herein, the “median” value (e.g., median neoantigen number, median neoantigen-microbial homology—e.g., median cross-reactivity score, median recognition potential score), or median activated T cell number-refers to the median value obtained from a population of subjects having a cancer (e.g., pancreatic cancer, e.g., PDAC). The median values may be previously determined reference values or may be contemporaneously determined values.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, or at least about 1000 cells expressing similar or different phenotypes.

As used herein, the terms “antibody” and “antibodies” refer to antigen-binding proteins of the immune system. As used herein, the term “antibody” includes whole, full-length antibodies having an antigen-binding region, and any fragment thereof in which the “antigen-binding portion” or “antigen-binding region” is retained, or single chains, for example, single chain variable fragment (scFv), thereof. The term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab. F(ab′)2, and Fab fragments that lack the Fc fragment of intact antibody, clear more rapidly from the circulation, and can have less non-specific tissue binding of an intact antibody (Wahl et al., J. Nucl. Med. 24:316-325 (1983). In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, C_{H} 1, C_{H} 2, and C_{H} 3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each VH and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1 q) of the classical complement system.

The term “antigen-binding portion,” “antigen-binding fragment,” or “antigen-binding region” of an antibody, as used herein, refers to that region or portion of an antibody that binds to the antigen and which confers antigen specificity to the antibody; fragments of antigen-binding proteins. It has been shown that the antigen-binding function of an antibody can be performed by fragments of a full-length antibody. Examples of antigen-binding portions encompassed within the term “antibody fragments” of an antibody include a Fab fragment, a monovalent fragment consisting of the V_{L}, V_{H}, C_{L} and C_{H}1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR).

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al. Hyrbidoma (Larchmt) 2008 27(6):455-51; Peter et al., J Cachexia Sarcopenia Muscle 2012 Aug. 12; Shich et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Mooscaner et al., Ther Immunol 1995 2(10:31-40). Agonistic scFvs having stimulatory activity have been described (see, e.g., Peter et al., J Bio Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “F (ab)” refers to a fragment of an antibody structure that binds to an antigen but is monovalent and does not have a Fc portion, for example, an antibody digested by the enzyme papain yields two F (ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “antigen-binding protein” refers to a protein or polypeptide that comprises an antigen-binding region or antigen-binding portion, that is, has a strong affinity to another molecule to which it binds. Antigen-binding proteins encompass antibodies, chimeric antigen receptors (CARs) and fusion proteins.

As used herein, the term “treating” or “treatment” refers to clinical intervention in an attempt to alter the disease course of the individual or cell being treated and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, and non-human animals (including, but not limited to, non-human primates, dogs, cats, rodents, horses, cows, pigs, mice, rats, hamsters, rabbits, and the like (e.g., which is to be the recipient of a particular treatment, or from whom cells are harvested). In certain embodiments, the subject is a human.

As used herein, an “effective amount” or “therapeutically effective amount” is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse, or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

As used herein, the term “response” or “responsiveness” refers to an alteration in a subject's condition that occurs as a result of or correlates with treatment. In certain embodiments, a response is a beneficial response. In certain embodiments, a beneficial response can include stabilization of the condition (e.g., prevention or delay of deterioration expected or typically observed to occur absent the treatment), amelioration (e.g., reduction in frequency and/or intensity) of one or more symptoms of the condition, and/or improvement in the prospects for cure of the condition, etc. In certain embodiments, “response” can refer to response of an organism, an organ, a tissue, a cell, or a cell component or in vitro system. In certain embodiments, a response is a clinical response. In certain embodiments, presence, extent, and/or nature of response can be measured and/or characterized according to particular criteria. In certain embodiments, such criteria can include clinical criteria and/or objective criteria. In certain embodiments, techniques for assessing response can include, but are not limited to, clinical examination, positron emission tomography, chest X-ray CT scan, MRI, ultrasound, endoscopy, laparoscopy, and/or presence or level of a particular marker in a sample, cytology, and/or histology. Where a response of interest is a response of a tumor to a therapy, ones skilled in the art will be aware of a variety of established techniques for assessing such response, including, for example, for determining tumor burden, tumor size, tumor stage, etc. The likelihood of a subject having predictive features identified herein (e.g., number of neoantigens, number of activated T cells) exhibiting a particular response is relative to a similarly situated second subject (e.g., a second subject having the same type cancer, optionally the same type of cancer with similar characteristics (e.g., stage and/or location/distribution)) or group of subjects that lack one or more predictive feature.

As used herein, the term “sample” refers to a biological sample obtained or derived from a source of interest, as described herein. In certain embodiments, a source of interest comprises an organism, such as an animal or human. In certain embodiments, a biological sample is a biological tissue or fluid. Non-limiting biological samples include bone marrow, blood, blood cells, ascites, (tissue or fine needle) biopsy samples, cell-containing body fluids, free floating nucleic acids, sputum, saliva, urine, cerebrospinal fluid, peritoneal fluid, pleural fluid, feces, lymph, gynecological fluids, swabs (e.g., skin swabs, vaginal swabs, oral swabs, and nasal swabs), washings or lavages such as a ductal lavages or broncheoalveolar lavages, aspirates, scrapings, specimens (e.g., bone marrow specimens, tissue biopsy specimens, and surgical specimens), feces, other body fluids, secretions, and/or excretions, and cells therefrom, etc.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “vaccine” refers to a composition for generating immunity for the prophylaxis and/or treatment of diseases (e.g., neoplasia/tumor). In certain embodiments, vaccines are medicaments that comprise antigens and are intended to be used in humans or animals for generating specific defense and protective substance by vaccination.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

The terminology used in the present disclosure is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

### 2. Neoantigens

The presently disclosed subject matter provides identification of neoantigens in subjects having cancer.

### 2.1 Neoantigens

Neoantigen is a tumor-specific antigen that arises from one or more tumor-specific mutation. In certain embodiments, the neoantigen is a tumor-specific antigen that arises from a tumor-specific mutation. In certain embodiments, a neoantigen is not expressed by healthy cells (e.g., non-tumor cells or non-cancer cells) in a subject.

Many antigens expressed by cancer cells are self-antigens which are selectively expressed or overexpressed on the cancer cells. These self-antigens are difficult to target with immunotherapy because they require overcoming both central tolerance (whereby autoreactive T cells are deleted in the thymus during development) and peripheral tolerance (whereby mature T cells are suppressed by regulatory mechanisms). Targeting neoantigens can abrogate these tolerance mechanisms. In certain embodiments, a neoantigen is recognized by cells of the immune system of a subject (e.g., T cells) as “non-self.” Neoantigens are not recognized as “self-antigens” by immune system, T cells that are capable of targeting neoantigens are not subject to central and peripheral tolerance mechanisms to the same extent as T cells which recognize self-antigens.

In certain embodiments, the tumor-specific mutation that results in a neoantigen is a somatic mutation. Somatic mutations comprise DNA alterations in non-germline cells and commonly occur in cancer cells. Certain somatic mutations in cancer cells result in the expression of neoantigens, that in certain embodiments, transform a stretch of amino acids from being recognized as “self” to “non-self.” Human tumors without a viral etiology can accumulate tens to hundreds of fold somatic mutations in tumor genes during neoplastic transformation, and some of these somatic mutations can occur in protein-coding regions and result in the formation of neoantigens. The exome is the protein-encoding part of the genome. Based on the mutations present within the protein-encoding part of the genome (i.e., the exome) of an individual tumor, potential neoantigens can be predicted for the individual tumor. In certain embodiments, whole exome sequencing is performed in a biological sample (e.g., a tumor sample) obtained from a subject having cancer.

In certain embodiments, a neoantigen is a neoantigenic peptide comprising a tumor specific mutation. The neoantigenic peptide can be a peptide that is incorporated into a larger protein. In certain embodiments, a neoantigenic peptide is a series of residues, typically L-amino acids, connected one to the other, typically by peptide bonds between the α-amino and carboxyl groups of adjacent amino acids. A neoantigenic peptide can be a variety of lengths, either in their neutral (uncharged) forms or in forms which are salts, and either free of modifications such as glycosylation, side chain oxidation, or phosphorylation.

In certain embodiments, the size of the neoantigenic peptide is about 3-30 amino acids, e.g., about 3-5, about 5-15 (e.g., about 8-11, about 5-10, or about 10-15), about 15-20, about 20-25, or about 20-30 amino acids, in length. In certain embodiments, the neoantigenic peptide is about 8-11 amino acids in length. In certain embodiments, the neoantigenic peptide is about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 amino acids in length. In certain embodiments, the neoantigenic peptide is at least about 3, at least about 5, or at least about 8 amino acids in length. In certain embodiments, the neoantigenic peptide is less than about 30, less than about 20, less than about 15, or less than about 10 amino acids in length.

Neoantigens may vary in different subjects, e.g., different subjects may have different combinations of neoantigens, also referred to as “neoantigen signatures.” For example, each subject may have a unique neoantigen signature.

The presently disclosed subject matter also provides compositions comprising one or more presently disclosed neoantigens. In certain embodiments, the compositions are pharmaceutical compositions comprising pharmaceutically acceptable carriers. In certain embodiments, the composition comprises two or more neoantigens. In some embodiments, a respective neoantigen is in the form of a peptide or protein. In some embodiments, a respective neoantigen is in the form of RNA. In some embodiments, a respective neoantigen is in the form of DNA. For instance, in some embodiments, a respective neoantigen is obtained as (e.g., encoded in) one or more RNA molecules and/or one or more DNA molecules. In some embodiments, two or more neoantigens (e.g., in peptide, RNA, and/or DNA form) can be linked, e.g., by any biochemical strategy to link two or more polymers (e.g., linking nucleic acids and/or proteins).

### 2.2 Detection of Neoantigens

Cancers can be screened to detect neoantigens using any of a variety of known technologies. In certain embodiments, neoantigens or expression thereof is detected at the nucleic acid level (e.g., in DNA or RNA). In certain embodiments, neoantigens or expression thereof is detected at the protein level (e.g., in a sample comprising polypeptides from cancer cells, which sample can be or comprise polypeptide complexes or other higher order structures including but not limited to cells, tissues, or organs).

In certain embodiments, a neoantigen is detected by the method selected from the group consisting of whole exome sequencing, immunoassay, microarray, genome sequencing, RNA sequencing, ELISA, Western Blotting, DNA or RNA sequencing, mass spectrometry, and combinations thereof. In certain embodiments, one or more neoantigens are detected by whole exome sequencing. In certain embodiments, one or more neoantigens are detected by the immunogenicity analysis method of somatic mutations described in Snyder et al. Engl J Med 371, 2189-2199 (2014). In certain embodiments, one or more neoantigens are detected by the pVAC-Seq method described in Hndal et al., Genome Med (2016); 8:11. In certain embodiments, one or more neoantigens are detected by the in silico neoantigen prediction pipeline method described in Rizvi et al., Science 348, 124-128 (2015). In certain embodiments, one or more neoantigens are detected by any of the methods described in WO2015/103037 and WO2016/081947. In some embodiments, one or more neoantigens are detected by any of the methods disclosed in, for instance, PCT Application No. PCT/US2018/014282, filed Jan. 18, 2018, entitled “Neoantigens and uses thereof for treating cancer,” which is hereby incorporated herein by reference in its entirety.

### 3. Uses of Neoantigens for Patient Selection for and Responsiveness Prediction to Immunotherapies

The neoantigens of the presently disclosed subject matter can be used to identify cancer subjects as candidates for immunotherapies, and to predict responsiveness of cancer subjects to immunotherapies. In certain embodiments, the presently disclosed subject matter provides methods of identifying subjects (e.g., cancer subjects) as candidates for treatment with an immunotherapy (hereinafter “the patient selection method”). In certain embodiments, the presently disclosed subject matter provides methods of predicting the responsiveness of subjects (e.g., cancer subjects) to an immunotherapy (hereinafter “the responsiveness prediction method”).

### 3.1 Immunogenic Hotspot

As used herein, the term “immunogenic hotspot” refers to a genetic locus that is enriched with neoantigens (e.g., a genetic locus that frequently generates neoantigens). The immunogenic hotspot can vary depending on the type of disease (e.g., tumor). Detecting one or more neoantigens of an immunogenic hotspot can be used to identify cancer subjects as candidates for immunotherapies, and to predict responsiveness of cancer subjects to immunotherapies. Furthermore, immunotherapies (e.g., vaccines, T cells (including modified T cells, e.g., T cells comprising a T cell receptor (TCR) or a chimeric antigen receptor (CAR)) targeting one or more neoantigens of an immunogenic hotspot can be used for neoantigen-directed therapies, e.g., cancer therapies.

### 3.2 Neoantigen Quantity and Neoantigen Immunogenicity

In certain embodiments, the patient selection method and responsiveness prediction method relate to the quantity of the neoantigens (e.g., the number of neoantigens (hereinafter “neoantigen number”) in the subject.

In some embodiments, neoantigen number is measured by any methods for detecting neoantigens, e.g., those described in Section 2.2. In certain embodiments, the neoantigen number is measured by whole exome sequencing the biological sample. In certain embodiments, the neoantigen number is measured by the immunogenicity analysis method of somatic mutations described in Snyder et al., 204, Engl J Med 371, 2189-2199. In certain embodiments, the neoantigen number is measured by the pVAC-Seq method described in Hndal et al., 2016, Genome Med 8, 11. In certain embodiments, the neoantigen number is measured by the in silico neoantigen prediction pipeline described in Rizvi et al., 2015 Science 348, 124-128. In certain embodiments, the neoantigen number is measured by any of the methods described in WO2015/103037 and WO2016/081947. In some embodiments, the neoantigen number is measured by any of the methods disclosed in, for instance, PCT Application No. PCT/US2018/014282, filed Jan. 18, 2018, entitled “Neoantigens and uses thereof for treating cancer,” which is hereby incorporated herein by reference in its entirety.

In certain embodiments, the patient selection method and responsiveness prediction method relate to the quantity of the neoantigens and the immunogenicity of the neoantigens (hereinafter “neoantigen immunogenicity”) in the subject. In certain embodiments, the patient selection method and responsiveness prediction method comprise assessing the neoantigen immunogenicity. Immunogenicity is the ability of a particular substance, such as an antigen (e.g., a neoantigen), to induce or stimulate an immune response in the cells expressing such antigen. In certain embodiments, assessing the neoantigen immunogenicity comprises measuring one or more surrogate for the neoantigen immunogenicity.

In certain embodiments, the surrogate is the homology between a neoantigen and a microbial epitope (hereinafter “neoantigen-microbial homology”).

In some embodiments, the cancer is a solid tumor. In some embodiments, the cancer is a liquid tumor. Non-limiting examples of solid tumor include pancreatic cancer, gastric cancer, bile duct cancer (e.g., cholangiocarcinoma), liver cancer, colorectal cancer, melanoma, lung cancer, and breast cancer. Non-limiting examples of liquid tumor include acute leukemia and chronic leukemia. In certain embodiments, the cancer is pancreatic cancer. In certain embodiments, the pancreatic cancer is pancreatic ductal adenocarcinoma (PDAC).

### 3.3 Immunotherapy

Immunotherapies that boost the ability of endogenous T cells to destroy cancer cells have demonstrated therapeutic efficacy in a variety of human malignancies. However, some cancer patients have resistance to certain immunotherapies. The presently disclosed subject matter provides methods for identifying cancer patients who would be candidates and/or who would likely to respond to an immunotherapy.

Non-limiting examples of immunotherapies include therapies comprising one or more immune checkpoint blocking antibody, adoptive T cell therapies, non-checkpoint blocking antibody-based immunotherapies, small molecule inhibitors, cancer vaccines, and combinations thereof.

Non-limiting examples of immune checkpoint blocking antibodies include antibodies against CTLA cytotoxic T-lymphocyte antigen 4 (anti-CTLA4 antibodies), antibodies against programmed death 1 (anti-PD-1 antibodies), antibodies against Programmed death-ligand 1 (anti-PD-L1 antibodies), antibodies against lymphocyte activation gene-3 (anti-LAG3 antibodies), antibodies against T cell immunoglobulin and mucin domain-containing protein 3 (anti-TIM-3 antibodies), antibodies against glucocorticoid-induced TNFR-related protein (GITR), antibodies against OX40, antibodies against CD40, antibodies against T cell immunoreceptor with Ig and ITIM domains (TIGIT), antibodies against 4-1BB, antibodies against B7 homolog 3 (anti-B7-H3 antibodies), antibodies against B7 homolog 4 (anti-B7-H4 antibodies), and antibodies against B- and T-lymphocyte attenuator (anti-BTLA antibodies).

Adoptive T cell therapy involves the isolation and ex vivo expansion of tumor specific T cells to achieve greater number of T cells. The tumor specific T cells are infused into cancer patients to give their immune system the ability to overwhelm remaining tumor via T cells which can attack and kill cancer. Non-limiting examples of adoptive T cell therapy include tumor-infiltrating lymphocyte (TIL) cell therapies, therapies comprising engineered or modified T cells, e.g., T cells engineered or modified with T cell receptor (TCR-transduced T cells), or T cells engineered or modified with chimeric antigen receptor (CAR-transduced T cells). These engineered or modified T cells recognize specific antigens associated with the cancers and attack cancers.

### 4. Therapeutic Uses of the Neoantigens

In some embodiments, the neoantigens of the present disclosure, or identified using the methods of the present disclosure, are used for vaccine therapy and/or adoptive T cell therapies.

### 4.1 Vaccines

Neoantigens can be an attractive source of targets for vaccine therapy. Neoantigen-based cancer vaccine can induce more robust and specific anti-tumor T cell responses compared with conventional shared-antigen-targeted vaccine. Certain genetic loci, also known as immunogenic hotspots, can be preferentially enriched for neoantigens in specific tumors that display great T cell infiltration and adaptive immune activation. Vaccine targeting tumor-specific immunogenic hotspot generated neoantigen can induce robust and specific anti-tumor T cell responses against the tumor cell. The vaccine can be used along or in combination with other cancer treatment, e.g., immunotherapy.

In one aspect, the present disclosure provides a vaccine comprising one or more of the presently disclosed neoantigens, or a polynucleotide encoding the neoantigen, or a protein or peptide comprising the neoantigen. In some embodiments, the neoantigen is selected based, at least in part, on predicted immunogenicity, for example, in silico. In some embodiments, the predicted immunogenicity is analyzed using computational algorithms for MHC class I and class II binding as well as use of tandem minigene libraries for class II epitope screening. In addition, in some embodiments, neoantigen specific T cell assays are used to differentiate true immunogenic neoepitopes from putative ones (see, Kvistborg et. al., 2016, J. ImmunoTherapy of Cancer 4:22 for detailed review). In some embodiments, any methods and tools known in the art are used to predict immunogenicity of a neoantigen. For example, in some embodiments, the Immune Epitope Database (IEDB) T Cell Epitope-MHC Binding Prediction Tool disclosed in Brown et. al. 2010, Nucleic Acids Res. Jan; 38 (Database issue): D854-62) is used to predict the binding of neoantigen to autologous HLA-A encoded MHC proteins. In certain embodiments, immunogenicity analysis strategy and tools disclosed in WO2015/103037 are used in accordance with the disclosed subject matter, and the content of the forgoing patent is incorporated herein by reference in its entirety. In certain embodiments, a neoantigen is selectively targeted based on one or more of the following: (i) homology to an epitope of a known pathogen or microbe and/or (ii) ability to activate T cells, e.g., in an in vitro assay.

In another aspect, the present disclosure provides a vaccine comprising one or more neoantigens identified using any of the neoantigen identification methods disclosed herein.

In certain embodiments, the vaccine comprises one or more neoantigens associated with a cancer, the one or more neoantigens correlating with a neoantigen-microbial homology that is higher than the median neoantigen-microbial homology occurring in subjects with the cancer, or a polynucleotide encoding the neoantigen or a protein or peptide comprising the neoantigen. In certain embodiments, the vaccine comprises one or more neoantigens associated with a cancer, the neoantigen correlating with an activated T cell number that is higher than the median activated T cell number occurring in subjects with a cancer, or a polynucleotide encoding the neoantigen or a protein or peptide comprising the neoantigen.

In certain embodiments, the vaccine comprises a neoantigen that occurs in a subject with a cancer, where this subject has an activated T cell number that is higher than the median activated T cell number of a population of subjects with the cancer. In certain embodiments, the neoantigen less frequently occurs in subjects with the cancer and having activated T cell numbers at or less than the median activated T cell number.

In some embodiments, the number of different neoantigens in the vaccine varies, for example, in some embodiments the vaccine comprises 2, 3, 4, 5, 6, 7, 8, 9, 10 or more different neoantigens. In some embodiments, the neoantigens of a given vaccine are linked, e.g., by any biochemical strategy to link two proteins or peptides. In some embodiments, the neoantigens of a given vaccine are not linked.

In certain embodiments, the vaccine comprises one or more polynucleotides. In some embodiments, the one or more polynucleotides are RNA, DNA, or a mixture thereof. In certain embodiments, the vaccine is in the form of DNA or RNA vaccines relating to neoantigens. In some embodiments, the one or more neoantigens are delivered via a bacterial or viral vector containing DNA or RNA sequences that encode one or more neoantigen.

Non-limiting examples of vaccines of the present disclosure include tumor cell vaccines, antigen vaccines, and dendritic cell vaccines, RNA vaccines, DNA vaccines, and/or viral vector-based vaccines. Tumor cell vaccines are made from cancer cells removed from the patient. In some such embodiments, the cells are altered (and killed) to make them more likely to be attacked by the immune system and then injected back into the patient. In some embodiments, the tumor cell vaccines are autologous, e.g., the vaccine is made from killed tumor cells taken from the same person who receives the vaccine. In some embodiments, the tumor cell vaccines are allogeneic, e.g., the cells for the vaccine come from someone other than the patient being treated.

In some embodiments, the vaccine is an antigen presenting cell vaccine, e.g., a dendritic cell vaccine. Dendritic cells are special immune cells in the body that help the immune system recognize cancer cells. They break down cancer cells into smaller pieces (including antigens), and then hold out these antigens so T cells can see them. The T cells then start an immune reaction against any cells in the body that contain these antigens.

In some embodiments, the antigen presenting cell such as a dendritic cell is pulsed or loaded with the neoantigen, or genetically modified (via DNA or RNA transfer) to express one or more neoantigens (see, e.g., Butterfield, 2015, BMJ. 22, 350; and Palucka, 2013, Immunity 39, 38-48). In certain embodiments, the dendritic cell is genetically modified to express one or more neoantigens. In some embodiments, any suitable method known in the art is used for preparing dendritic cell vaccines of the present disclosure. For example, in some embodiments, immune cells are removed from the patient's blood and exposed to cancer cells or cancer antigens, as well as to other chemicals that turn the immune cells into dendritic cells and help them grow. The dendritic cells are then injected back into the patient, where they can cause an immune response to cancer cells in the body.

Furthermore, the presently disclosed subject matter provides methods for treating pancreatic cancer in a subject. In certain embodiments, the method comprises administering to the subject a presently disclosed vaccine.

In certain embodiments, the vaccination is therapeutic vaccination, administered to a subject who has pancreatic cancer. In certain embodiments, the vaccination is prophylactic vaccination, administered to a subject who can be at risk of developing pancreatic cancer. In certain embodiments, the vaccine is administered to a subject who has previously had cancer and in whom there is a risk of the cancer recurring.

Vaccines can be administered in any suitable way as known in the art. In certain embodiments, the vaccine is delivered using a vector delivery system. In some embodiments, the vector delivery system is viral, bacterial or makes use of liposomes. In certain embodiments, a listeria vaccine or electroporation is used to deliver the vaccine.

The presently disclosed subject matter further provides a composition comprising a presently disclosed vaccine. In certain embodiments, the composition is a pharmaceutical composition. In some embodiments, the pharmaceutical composition further comprises a pharmaceutically acceptable carrier, diluent, or excipient.

In certain embodiments, the vaccine leads to generation of an immune response in a subject. In some embodiments, the immune response is humoral and/or cell-mediated immunity, for example the stimulation of antibody production, or the stimulation of cytotoxic or killer cells, which can recognize and destroy (or otherwise eliminate) cells (e. g., tumor cells) expressing antigens (e.g., neoantigen) corresponding to the antigens in the vaccine on their surface. In some embodiments, inducing or stimulating an immune response includes all types of immune responses and mechanisms for stimulating them. In certain embodiments, the induced immune response comprises expansion and/or activation of Cytotoxic T Lymphocytes (CTLs). In certain embodiments, the induced immune response comprises expansion and/or activation of CD8⁺ T cells. In certain embodiments, the induced immune response comprises expansion and/or activation of helper CD4+ T Cells. In some embodiments, the extent of an immune response is assessed by production of cytokines, including, but not limited to, IL-2, IFN-γ, and/or TNFα.

### 4.2 Adoptive T Cell Therapy

In some embodiments of the present disclosure, the neoantigens of the present disclosure are used in adoptive T cell therapy. The presently disclosed subject matter provides a population of T cells that target one or more of the presently disclosed neoantigens. In some embodiments, the neoantigen are selected based, at least in part, on predicted immunogenicity, for example, in silico. In some embodiments, the predicted immunogenicity is analyzed using computational algorithms for MHC class I and class II binding as well as use of tandem minigene libraries for class II epitope screening. In addition, or alternatively, in some embodiments neoantigen specific T cell assays are used to differentiate true immunogenic neoepitopes from putative ones (see, Kvistborg et. al., 2016, J. ImmunoTherapy of Cancer 4:22 for a detailed review). In some embodiments, any methods and tools known in the art are used to predict immunogenicity of a neoantigen. For example, in some embodiments the Immune Epitope Database (IEDB) T Cell Epitope-MHC Binding Prediction Tool disclosed in Brown et. al., 2010, Nucleic Acids Res. 38 (Database issue): D854-62 is used to predict the binding of neoantigen to autologous HLA-A encoded MHC proteins. In certain embodiments, immunogenicity analysis strategy and tools disclosed in WO2015/103037 are used in accordance with the disclosed subject matter, and the content of the forgoing patent is incorporated herein by reference in its entirety. In certain embodiments, a neoantigen is selectively targeted based on one or more of the following: (i) homology to an epitope of a known pathogen or microbe; and/or (ii) ability to activate T cells, e.g., in an in vitro assay.

In certain embodiments, the population of T cells target one or more neoantigens associated with a cancer, the one or more neoantigens correlating with a neoantigen-microbial homology that is higher than the median neoantigen-microbial homology occurring in subjects with the cancer. In certain embodiments, the population of T cells target one or more neoantigens associated with a cancer, the neoantigen correlating with an activated T cell number that is higher than the median activated T cell number occurring in subjects with the cancer. In certain embodiments, the neoantigen comprised in the vaccine occurs in a subject with a cancer, where the subject has an activated T cell number that is higher than the median activated T cell number of a population of subjects with the cancer. In certain embodiments, the neoantigen occurs less frequently in subjects with the cancer and having activated T cell numbers at or less than the median activated T cell number.

In certain embodiments, the T cells are selectively expanded to target the one or more neoantigen. T cells are lymphocytes that mature in the thymus and are chiefly responsible for cell-mediated immunity. T cells are involved in the adaptive immune system. In some embodiments, the T cells of the presently disclosed subject matter are any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, regulatory T cells (also known as suppressor T cells), natural killer T cells, mucosal associated invariant T cells, and γδ T cells. Cytotoxic T cells (CTL or killer T cells) are a subset of T lymphocytes capable of inducing the death of infected somatic or tumor cells.

In certain embodiments, the T cells that specifically target one or more neoantigens are engineered or modified T cells. In certain embodiments, the engineered T cells comprise a recombinant antigen receptor that specifically targets or binds to one or more of the presently disclosed neoantigens. In certain embodiments, the recombinant antigen receptor specifically targets one or more neoantigens associated with a cancer, the one or more neoantigens correlating with a neoantigen-microbial homology that is higher than the median neoantigen-microbial homology occurring in subjects with the cancer. In certain embodiments, the recombinant antigen receptor specifically targets one or more neoantigens associated with a cancer, the neoantigen correlating with an activated T cell number that is higher than the median activated T cell number occurring in subjects with the cancer. In certain embodiments, the recombinant antigen receptor is a chimeric antigen receptor (CAR). In certain embodiments, the recombinant antigen receptor is a T cell receptor (TCR). In certain embodiments, the CAR comprises an extracellular antigen-binding domain that specifically binds to one or more neoantigen, a transmembrane domain, and an intracellular signaling domain. CARs can activate the T cell in response to recognition by the extracellular antigen-binding domain of its target. When T cells express such a CAR, they recognize and kill cells that express one or more neoantigen.

Affinity-enhanced TCRs are generated by identifying a T cell clone from which the TCR α and β chains with the desired target specificity are cloned. The candidate TCR then undergoes PCR directed mutagenesis at the complimentary determining regions (“CDR”) of the α and β chains. The mutations in each CDR region are screened to select for mutants with enhanced affinity over the native TCR. Once completed, lead candidates are cloned into vectors to allow functional testing in T cells expressing the affinity-enhanced TCR.

In certain embodiments, the T cell population is enriched with T cells that are specific to one or more neoantigen, e.g., having an increased number of T cells that target one or more neoantigen. Therefore, the T cell population differs from a naturally occurring T cell population, in that the percentage or proportion of T cells that target a neoantigen is increased.

In certain embodiments, the T cell population comprises at least about 10%, about 20%, about 30%, about 40%, about 50%, about 60%, about 70%, about 80%, about 90%, about 95%, or about 100% T cells that target one or more neoantigen. In certain embodiments, the T cell population comprises no more than about 5%, about 10%, about 15%, about 20%, about 25%, about 30%, about 35%, about 40%, about 45%, or about 50% T cells that do not target one or more neoantigen.

In certain embodiments, the T cell population is generated from T cells isolated from a subject with cancer. In one non-limiting example, the T cell population is generated from T cells in a biological sample isolated from a subject with cancer. In some embodiments, the biological sample is a tumor sample, a peripheral blood sample, or a sample from a tissue of the subject. In certain embodiments, the T cell population is generated from a biological sample in which the one or more neoantigens are identified or detected.

The presently disclosed subject matter further provides a composition comprising such T cell populations as described herein. In certain embodiments, the composition is a pharmaceutical composition that comprises a pharmaceutically acceptable carrier. Furthermore, the presently disclosed subject matter provides a method of treating cancer in a subject, comprising administering to the subject a composition comprising such T cell population as described herein. In some embodiments, the cancer is any of the cancers enumerated in the present disclosure. In some embodiments the cancer is pancreatic cancer.

In some embodiments, the methods are used in vitro, ex vivo or in vivo, for example, either for in situ treatment or for ex vivo treatment followed by the administration of the treated cells to the subject.

### 5. Example Embodiments for Identifying a Tumor Vaccine Personalized to a Human Subject

### 5.1 Example Systems for Identifying a Tumor Vaccine Personalized to a Human Subject

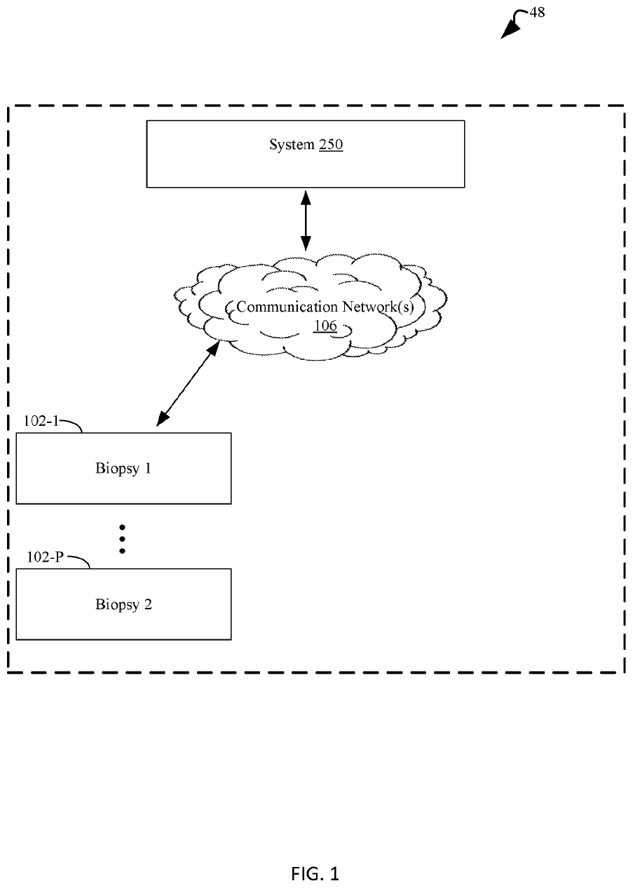

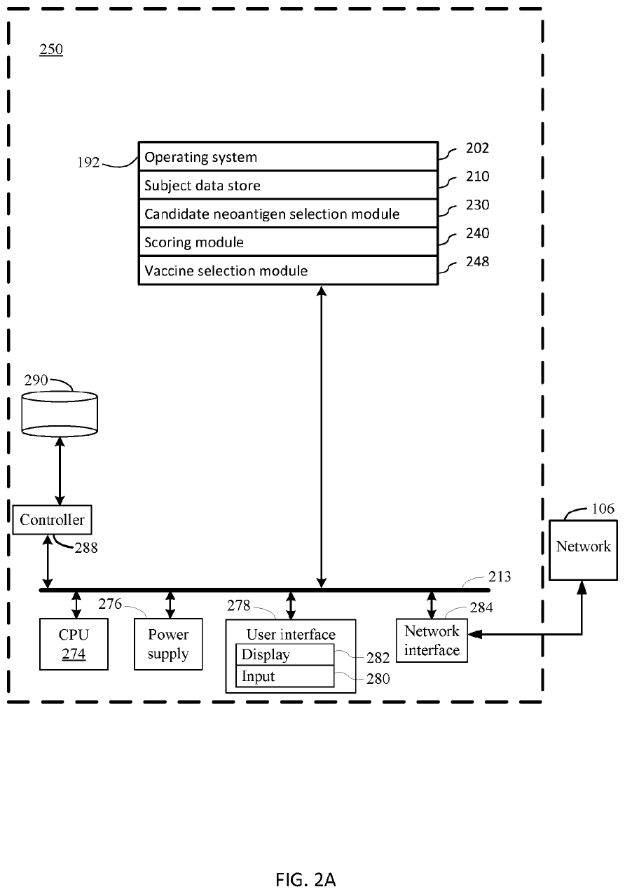

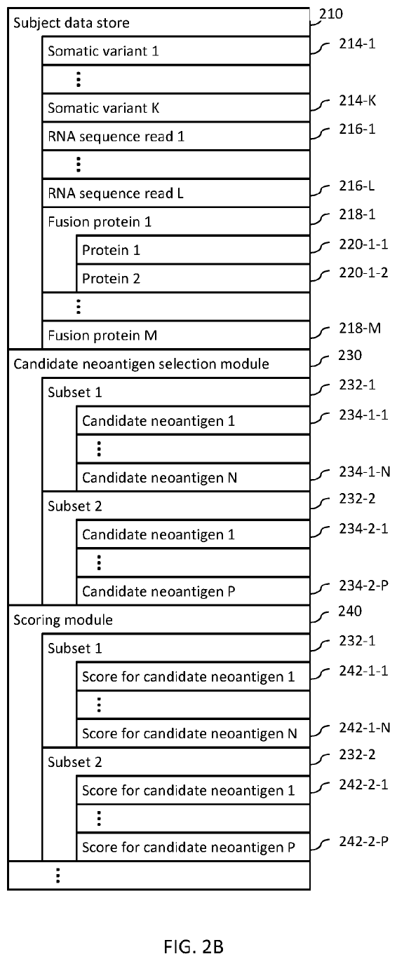

One aspect of the present disclosure provides systems for identifying a tumor vaccine personalized to a human subject afflicted with a cancer. FIG. 1 illustrates an example of an integrated system topology 48 for the acquisition of associated data, and FIGS. 2A-B provides more details of a system 250. The integrated system topology 48 includes data (e.g., sequence reads, somatic variants, etc.) from one or more samples 102 from a human cancer subject that is representative of the cancer, one or more communication networks 106, and a system (e.g., device) 250.

A detailed description of a system 250 for identifying a tumor vaccine personalized to a human subject afflicted with a cancer in accordance with the present disclosure) is described in conjunction with FIGS. 1 and 2A-B. As such, FIGS. 1 and 2A-B collectively illustrate the topology of the system in accordance with the present disclosure. In the topology, there is a device 250 for identifying a tumor vaccine personalized to a human subject afflicted with a cancer.

Referring to FIGS. 2A-B, the device 250 identifies a tumor vaccine personalized to a human subject afflicted with a cancer. In some embodiments, the device 250 receives data directly or indirectly through radio-frequency signals. In some embodiments such signals are in accordance with an 802.11 (WiFi), Bluetooth, or ZigBee standard. In some embodiments the device 250 receives data across one or more communications networks.

Examples of networks 106 include, but are not limited to, the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

Of course, other topologies of the system 48 of FIG. 1 are possible. For instance, rather than relying on a communications network 106, information may be sent directly to the device 250. Further, the device 250 may constitute a portable electronic device, a server computer, or in fact constitute several computers that are linked together in a network or be a virtual machine in a cloud computing context. As such, the exemplary topology shown in FIG. 1 merely serves to describe the features of an embodiment of the present disclosure in a manner that will be readily understood to one of skill in the art.

Referring to FIGS. 2A-B, in typical embodiments, the device 250 comprises one or more computers. For purposes of illustration in FIGS. 2A-B, the device 250 is represented as a single computer that includes all of the functionality for identifying a tumor vaccine personalized to a human subject afflicted with a cancer. However, the disclosure is not so limited. In some embodiments, the functionality for identifying a tumor vaccine personalized to a human subject afflicted with a cancer is spread across any number of networked computers and/or resides on each of several networked computers and/or is hosted on one or more virtual machines at a remote location accessible across the communications network 106. One of skill in the art will appreciate that any of a wide array of different computer topologies are used for the application and all such topologies are within the scope of the present disclosure.

Turning to FIGS. 2A-B with the foregoing in mind, an exemplary device 250 for identifying a tumor vaccine personalized to a human subject afflicted with a cancer comprises one or more processing units (CPU's) 274, a network or other communications interface 284, a memory 192 (e.g., random access memory), one or more magnetic disk storage and/or persistent devices 290 optionally accessed by one or more controllers 288, one or more communication busses 213 for interconnecting the aforementioned components, a user interface 278, the user interface 278 including a display 282 and input 280 (e.g., keyboard, keypad, touch screen), and a power supply 276 for powering the aforementioned components. In some embodiments, the input 280 is a touch-sensitive display, such as a touch-sensitive surface. In some embodiments, the user interface 278 includes one or more soft keyboard embodiments. The soft keyboard embodiments may include standard (QWERTY) and/or non-standard configurations of symbols on the displayed icons. In some embodiments, data in memory 192 is seamlessly shared with non-volatile memory 290 using known computing techniques such as caching. In some embodiments, memory 192 and/or memory 290 includes mass storage that is remotely located with respect to the central processing unit(s) 274. In other words, some data stored in memory 192 and/or memory 290 may in fact be hosted on computers that are external to the device 250 but that can be electronically accessed by the device 250 over an Internet, intranet, or other form of network or electronic cable (illustrated as element 106 in FIGS. 2A-B) using network interface 284.

In some embodiments, the memory 192 of the device 250 for identifying a tumor vaccine personalized to a human subject afflicted with a cancer includes:
an optional operating system 202 that includes procedures for handling various basic system services;
a subject data store 210, optionally including:
   a first plurality of somatic variants 214 (e.g., 214-1, . . . 214-K) of the subject,
   a first plurality of sequence reads 216 (e.g., 216-1, . . . 216-L) from RNA molecules in a sample of a tumor obtained from the subject, and
   one or more fusion proteins 218 (e.g., 218-1, . . . 218-M) encoded by the first plurality of sequence reads 216, where each respective fusion protein in the one or more fusion proteins is a fusion of a portion of a respective first human protein 220-1 (e.g., 220-1-1) and a portion of a respective second human protein 220-2 (e.g., 220-1-2);
a candidate neoantigen selection module 230, optionally including a first subset of candidate neoantigens 232-1 and a second subset of candidate neoantigens 232-2, where each neoantigen in the first subset of candidate neoantigens 234-1 (e.g., 234-1-1, . . . 234-1-N) encodes a somatic variant 214 in the first plurality of somatic variants, and each neoantigen in the second subset of candidate neoantigens 234-2 (e.g., 234-2-1, . . . 234-2-P) encodes one or more residues from the portion of the respective first human protein 220-1 and one or more residues from the portion of the respective second human protein 220-2;
a scoring module 240, optionally including, for each respective candidate neoantigen 234 in the plurality of candidate neoantigens, a respective score 242 (e.g., 242-1-1, . . . 242-1-N, . . . 242-2-1, . . . 242-2-P) determined using a scoring function that includes a first scoring term that upweights respective candidate neoantigens having a higher class I major histocompatibility complex (MHC) affinity relative to respective candidate neoantigens having a lower class I MHC affinity, given a class I human leukocyte antigen (HLA) type of the human subject; and
a vaccine selection module 248 that optionally selects, for the tumor vaccine, two or more candidate neoantigens 234 in the plurality of candidate neoantigens as a final set of neoantigens based on the respective score 242 of each candidate neoantigen in the plurality of candidate neoantigens.

In some embodiments, the subject assessment module 204 is accessible within any browser (phone, tablet, laptop/desktop). In some embodiments the subject assessment module 204 runs on native device frameworks, and is available for download onto the device 250 running an operating system 202 such as Android or iOS.

In some implementations, one or more of the above identified data elements or modules of the device 250 for identifying a tumor vaccine personalized to a human subject afflicted with a cancer are stored in one or more of the previously described memory devices, and correspond to a set of instructions for performing a function described above. The above-identified data, modules, or programs (e.g., sets of instructions) need not be implemented as separate software programs, procedures or modules, and thus various subsets of these modules may be combined or otherwise re-arranged in various implementations. In some implementations, the memory 192 and/or 290 optionally stores a subset of the modules and data structures identified above. Furthermore, in some embodiments the memory 192 and/or 290 stores additional modules and data structures not described above. Further still, in some embodiments, the device 250 stores data for identifying a tumor vaccine personalized to a human subject afflicted with a cancer for two or more subjects, five or more subjects, one hundred or more subjects, or 1000 or more subjects.

In some embodiments, a device 250 for identifying a tumor vaccine personalized to a human subject afflicted with a cancer is a smart phone (e.g., an iPHONE), laptop, tablet computer, desktop computer, or other form of electronic device (e.g., a gaming console). In some embodiments, the device 250 is not mobile. In some embodiments, the device 250 is mobile.

It should be appreciated that the device 250 illustrated in FIGS. 2A-B is only one example of a multifunction device that may be used for identifying a tumor vaccine personalized to a human subject afflicted with a cancer, and that the device 250 optionally has more or fewer components than shown, optionally combines two or more components, or optionally has a different configuration or arrangement of the components. The various components shown in FIGS. 2A-B are implemented in hardware, software, firmware, or a combination thereof, including one or more signal processing and/or application specific integrated circuits.

In some embodiments, the device 250 has any or all of the circuitry, hardware components, and software components found in the device 250 depicted in FIGS. 2A-B. In the interest of brevity and clarity, only a few of the possible components of the device 250 are shown in order to better emphasize the additional software modules that are installed on the device 250.

While the integrated system 48 disclosed in FIGS. 1 and 2A-B can work standalone, in some embodiments it can also be linked with electronic medical records to exchange information in any way.

### 5.2 Example Methods for Identifying a Tumor Vaccine Personalized to a Human Subject

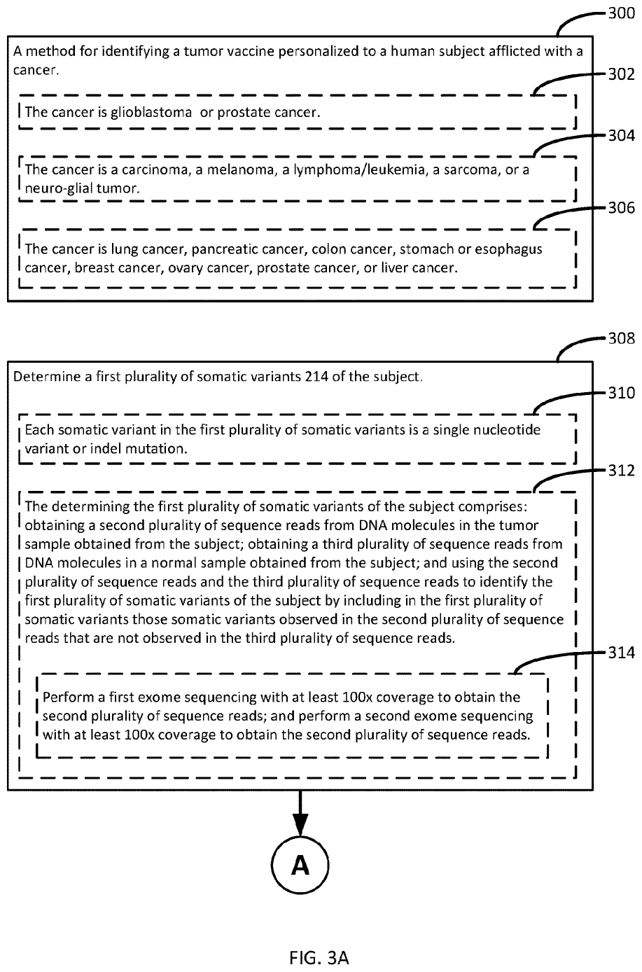

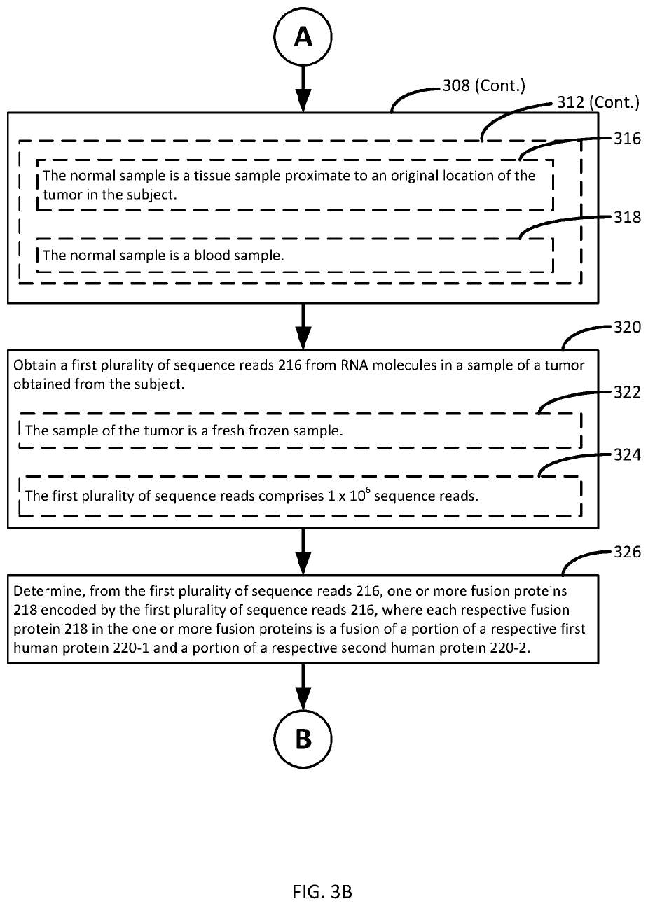

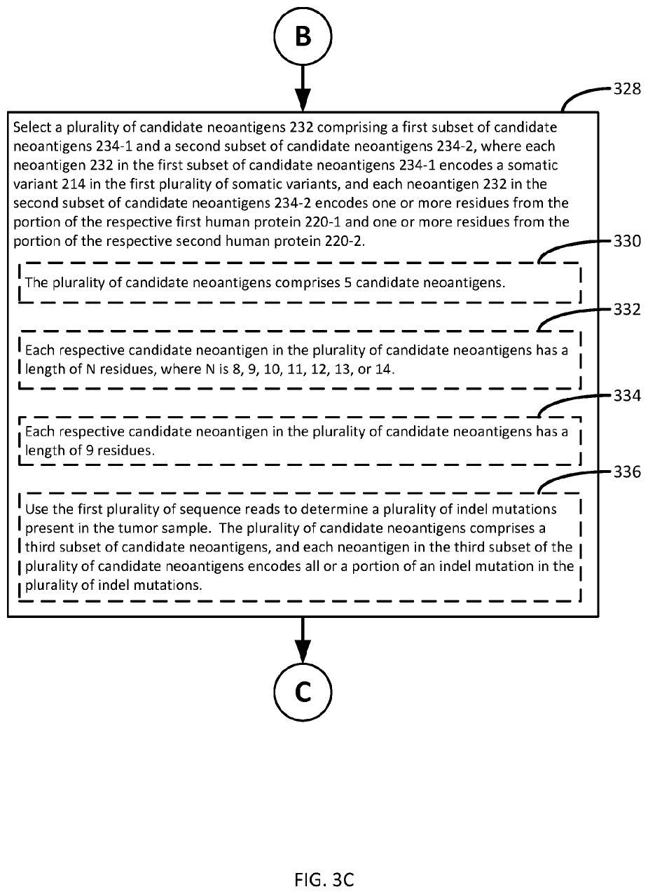

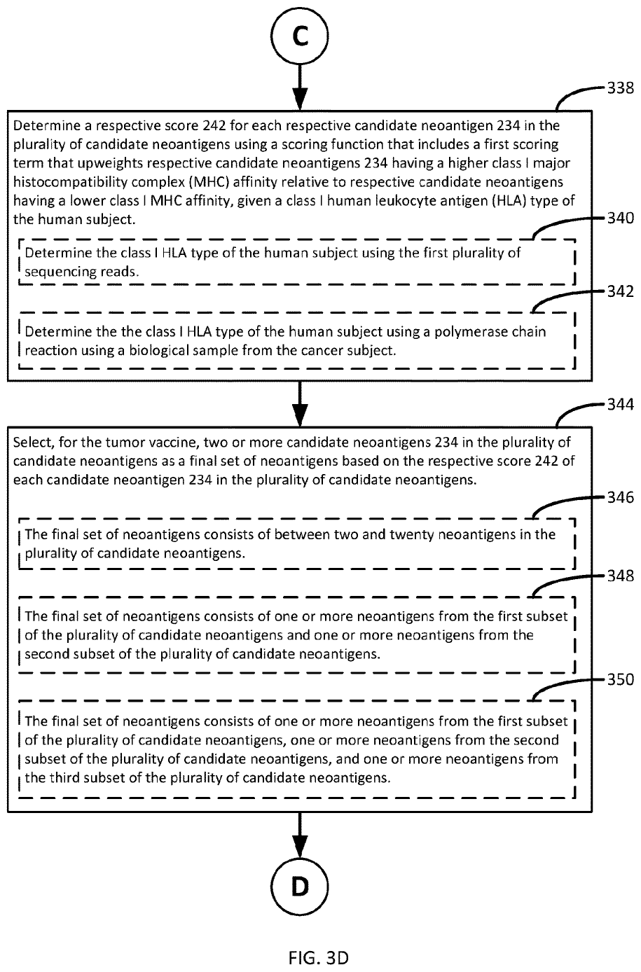

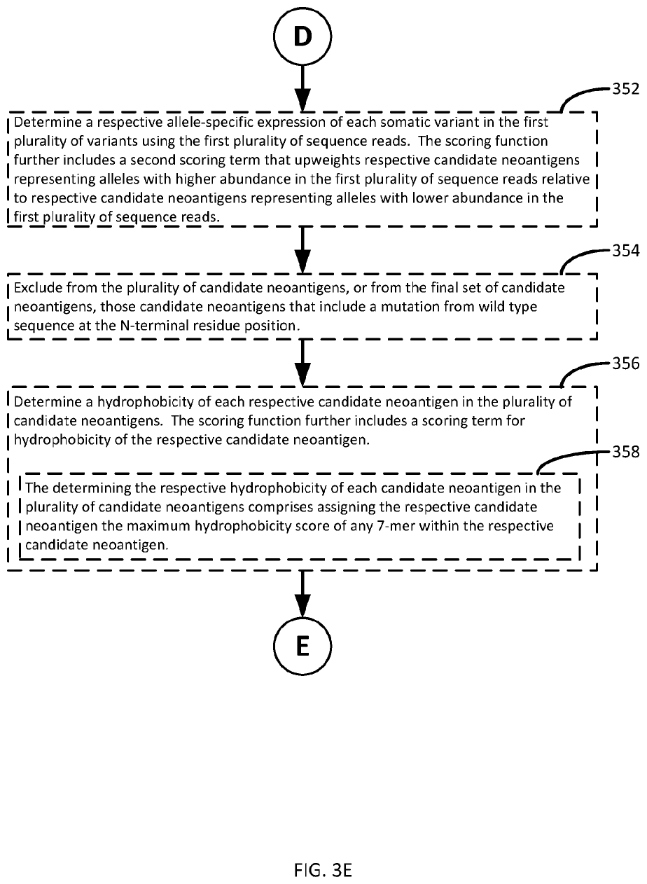

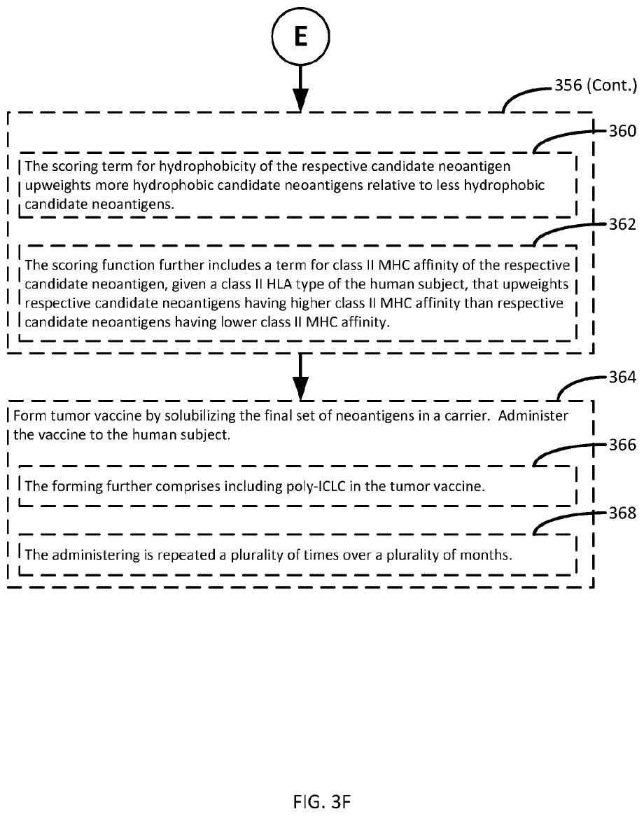

Now that details of an integrated system topology 48 for identifying a tumor vaccine personalized to a human subject afflicted with a cancer have been disclosed, details regarding a flow chart of processes and features of the system, in accordance with an embodiment of the present disclosure, are disclosed with reference to FIGS. 3A-F. In some embodiments, such processes and features of the system are conducted by the device 250 illustrated in FIGS. 2A-B.

FIGS. 3A-F collectively illustrate a method 300 for identifying a tumor vaccine personalized to a human subject afflicted with a cancer.

Referring to block 302, the cancer is glioblastoma or prostate cancer. Examples of target proteins (e.g., neoantigens) for tumor vaccines personalized to subjects afflicted with prostate cancer are described, for instance, in Examples 2-4 below.

Referring to block 304, in some embodiments, the cancer is a carcinoma, a melanoma, a lymphoma/leukemia, a sarcoma, or a neuro-glial tumor.

Referring to block 306, in some embodiments, the cancer is lung cancer, pancreatic cancer, colon cancer, stomach or esophagus cancer, breast cancer, ovary cancer, prostate cancer, or liver cancer. In some embodiments, the cancer is selected from the group consisting of bladder, breast, lung cancer, multiple myeloma, and head neck cancer.

In some embodiments, the cancer is a solid tumor. In some embodiments, the cancer is a liquid tumor. Non-limiting examples of solid tumor include pancreatic cancer, gastric cancer, bile duct cancer (e.g., cholangiocarcinoma), liver cancer, colorectal cancer, melanoma, lung cancer, and breast cancer. Non-limiting examples of liquid tumor include acute leukemia and chronic leukemia. In certain embodiments, the cancer is pancreatic cancer. In certain embodiments, the pancreatic cancer is pancreatic ductal adenocarcinoma (PDAC).

In some embodiments, the human subject is at risk of a cancer. In some embodiments, the human subject that is afflicted with a cancer has been diagnosed with the cancer. In some embodiments, determination of subjects afflicted with or at risk of cancer are made by any objective or subjective determination by a diagnostic test or opinion of a subject or health care provider. For example, numerous prognostic markers or factors for categorizing cancer patients or individuals for likely outcome of treatment are known. See, e.g., Lin P S & Semrad T J, Methods Mol Biol. 2018 1765:281-297; and Zacharakis et al., Anticancer Res, 2010 30(2): 653-660.

### Samples.

Referring to block 308, the method includes determining a first plurality of somatic variants 214 of the subject.

In some embodiments, the first plurality of somatic variants of the subject includes at least 2, at least 5, at least 10, at least 20, at least 50, at least 100, at least 200, at least 500, or at least 800 somatic variants. In some embodiments, the first plurality of somatic variants of the subject includes no more than 1000, no more than 800, no more than 500, no more than 200, no more than 100, no more than 50, no more than 20, no more than 10, or no more than 5 somatic variants. In some embodiments, the first plurality of somatic variants of the subject consists of from 2 to 10, from 2 to 50, from 5 to 80, from 50 to 200, from 100 to 500, or from 300 to 1000 somatic variants. In some embodiments, the first plurality of somatic variants of the subject falls within another range starting no lower than 2 somatic variants and ending no higher than 1000 somatic variants.

Referring to block 310, in some embodiments, each somatic variant in the first plurality of somatic variants is a single nucleotide variant or indel mutation.

In some embodiments, a respective somatic variant in the first plurality of somatic variants is any of the mutations and/or somatic variants disclosed herein (see, e.g., the section entitled “1. Definitions: Mutations,” above). For instance, in some embodiments, each respective somatic variant in the first plurality of somatic variants is a respective frameshift mutation.

Referring to block 320, the method includes obtaining a first plurality of sequence reads 216 from RNA molecules in a sample of a tumor obtained from the subject.

In some embodiments, any suitable method for obtaining biological samples, such as tumor samples, known in the art are contemplated for use in the present disclosure. In some implementations, the sample of the tumor is a tissue biopsy sample. In some embodiments, the sample of the tumor is a formalin-fixed tissue (FFT), such as a formalin-fixed paraffin-embedded (FFPE) tissue. In some embodiments, the tissue biopsy sample is an FFPE or FFT block.

In some embodiments, the sample of the tumor is a cryo-section of a tissue biopsy and/or a core needle biopsy.

Referring to block 322, in some embodiments, the sample of the tumor is a fresh frozen sample. In some embodiments, the sample of the tumor is OCT-embedded. Generally, OCT (Optimal Cutting Temperature) embedding refers to an embedding medium for embedding frozen tissue and is a procedure known in the art. In some embodiments, use of OCT medium prevents the formation of freezing artifacts that cause damage to tissue. In some embodiments, OCT medium or a similar medium according to general knowledge is used to embed a tissue sample (e.g., the sample of the tumor) before sectioning (e.g., on a cryostat).

In some embodiments, the sample of the tumor is prepared in thin sections (e.g., by cutting and/or affixing to a slide), to facilitate pathology review (e.g., by staining with immunohistochemistry stain for IHC review and/or with hematoxylin and eosin stain for H&E pathology review).

In certain embodiments, the sample of the tumor is a biological tissue or fluid. Non-limiting biological samples include bone marrow, blood, blood cells, ascites, (tissue or fine needle) biopsy samples, cell-containing body fluids, free floating nucleic acids, sputum, saliva, urine, cerebrospinal fluid, peritoneal fluid, pleural fluid, feces, lymph, gynecological fluids, swabs (e.g., skin swabs, vaginal swabs, oral swabs, and nasal swabs), washings or lavages such as a ductal lavages or broncheoalveolar lavages, aspirates, scrapings, specimens (e.g., bone marrow specimens, tissue biopsy specimens, and surgical specimens), feces, other body fluids, secretions, and/or excretions, and cells therefrom.

In some embodiments, the RNA molecules are extracted from the sample of the tumor. Methods for isolating nucleic acids from biological samples are known in the art and are dependent upon the type of nucleic acid being isolated (e.g., cfDNA, DNA, and/or RNA) and the type of sample from which the nucleic acids are being isolated (e.g., liquid biopsy samples, white blood cell buffy coat preparations, fresh tissue, formalin-fixed paraffin-embedded (FFPE) solid tissue samples, and fresh frozen solid tissue samples). The selection of any particular nucleic acid isolation technique for use in conjunction with the embodiments described herein is well within the skill of the person having ordinary skill in the art, who will consider the sample type, the state of the sample, the type of nucleic acid being sequenced to obtain a respective plurality of sequence reads, and/or the sequencing technology being used. Moreover, methods for sequencing nucleic acids from biological samples are known in the art, including but not limited to DNA (e.g., whole exome or genome sequencing (WES or WGS)) and/or RNA sequencing (RNA-seq). In some embodiments, the nucleic acid molecules are sequenced using targeted sequencing.

Referring to block 324, in some embodiments, the first plurality of sequence reads comprises 1×10⁶ sequence reads.

In some embodiments, the first plurality of sequence reads comprises at least 100,000, at least 500,000, at least 1×10⁶, at least 2×10⁶, at least 5×10⁶, at least 1×10⁷, at least 2×10⁷, at least 5×10⁷, at least 1×10⁸, at least 2×10⁸, at least 5×10⁸, or at least 1×10⁹ sequence reads. In some embodiments, the first plurality of sequence reads comprises no more than 2×10⁹, no more than 1×10⁹, no more than 5×10⁸, no more than 1×10⁸, no more than 5×10⁷, no more than 1×10⁷, no more than 5×10⁶, no more than 1×10⁶, or no more than 500,000 sequence reads. In some embodiments, the first plurality of sequence reads consists of from 100,000 to 1×10⁶, from 500,000 to 5×10⁶, from 1×10⁶ to 2×10⁷, from 2×10⁷ to 1×10⁸, from 5×10⁷ to 5×10⁸, or from 5×10⁸ to 2×10⁹ sequence reads. In some embodiments, the first plurality of sequence reads falls within another range starting no lower than 100,000 sequence reads and ending no higher than 2×10⁹ sequence reads.

In some embodiments, the first plurality of sequence reads encompasses a subset of a transcriptome for the sample of the tumor.

In some embodiments, the subset of the transcriptome is at least 1 percent, at least 10 percent, at least 20 percent, at least 30 percent, at least 40 percent, at least 50 percent, at least 70 percent, at least 80 percent, at least 90 percent, at least 95 percent, or at least 99 percent of the transcriptome for the sample of the tumor. In some embodiments, the subset of the transcriptome is no more than 100 percent, no more than 99 percent, no more than 95 percent, no more than 90 percent, no more than 80 percent, no more than 50 percent, no more than 30 percent, or no more than 10 percent of the transcriptome for the sample of the tumor. In some embodiments, the subset of the transcriptome is between 1 percent and 10 percent, between 5 percent and 15 percent, between 10 percent and 20 percent, between 15 percent and 30 percent, between 25 percent and 50 percent, between 45 percent and 75 percent, or between 70 percent and 100 percent of the transcriptome of the sample of the tumor. In some embodiments, the subset of the transcriptome falls within another range starting no lower than 1 percent and ending no higher than 100 percent of the transcriptome of the sample of the tumor.

In some embodiments, the method further includes obtaining a second plurality of sequence reads from DNA molecules in the sample of a tumor obtained from the subject.

In some embodiments, the method further includes obtaining a third plurality of sequence reads from DNA molecules in normal sample obtained from the subject.

### Determining Somatic Variants.

Referring to block 312, in some embodiments, the determining the first plurality of somatic variants of the subject includes obtaining a second plurality of sequence reads from DNA molecules in the tumor sample obtained from the subject; obtaining a third plurality of sequence reads from DNA molecules in a normal sample obtained from the subject; and using the second plurality of sequence reads and the third plurality of sequence reads to identify the first plurality of somatic variants of the subject by including in the first plurality of somatic variants those somatic variants observed in the second plurality of sequence reads that are not observed in the third plurality of sequence reads.

In some embodiments, the second plurality of sequence reads comprises at least 100,000, at least 500,000, at least 1×10⁶, at least 2×10⁶, at least 5×10⁶, at least 1×10⁷, at least 2×10⁷, at least 5×10⁷, at least 1×10⁸, at least 2×10⁸, at least 5×10⁸, or at least 1×10⁹ sequence reads. In some embodiments, the second plurality of sequence reads comprises no more than 2×10⁹, no more than 1×10⁹, no more than 5×10⁸, no more than 1×10⁸, no more than 5×10⁷, no more than 1×10⁷, no more than 5×10⁶, no more than 1×10⁶, or no more than 500,000 sequence reads. In some embodiments, the second plurality of sequence reads consists of from 100,000 to 1×10⁶, from 500,000 to 5×10⁶, from 1×10⁶ to 2×10⁷, from 2×10⁷ to 1×10⁸, from 5×10⁷ to 5×10⁸, or from 5×10⁸ to 2×10⁹ sequence reads. In some embodiments, the second plurality of sequence reads falls within another range starting no lower than 100,000 sequence reads and ending no higher than 2×10⁹ sequence reads.

In some embodiments, the third plurality of sequence reads comprises at least 100,000, at least 500,000, at least 1×10⁶, at least 2×10⁶, at least 5×10⁶, at least 1×10⁷, at least 2×10⁷, at least 5×10⁷, at least 1×10⁸, at least 2×10⁸, at least 5×10⁸, or at least 1×10⁹ sequence reads. In some embodiments, the third plurality of sequence reads comprises no more than 2×10⁹, no more than 1×10⁹, no more than 5×10⁸, no more than 1×10⁸, no more than 5×10⁷, no more than 1×10⁷, no more than 5×10⁶, no more than 1×10⁶, or no more than 500,000 sequence reads. In some embodiments, the third plurality of sequence reads consists of from 100,000 to 1×10⁶, from 500,000 to 5×10⁶, from 1×10⁶ to 2×10⁷, from 2×10⁷ to 1×10⁸, from 5×10⁷ to 5×10⁸, or from 5×10⁸ to 2×10⁹ sequence reads. In some embodiments, the third plurality of sequence reads falls within another range starting no lower than 100,000 sequence reads and ending no higher than 2×10⁹ sequence reads.

In some embodiments, a respective (e.g., second or third) plurality of sequence reads encompasses a respective subset of the genome of the subject (e.g., in a respective sample) and not the rest of the genome of the subject (e.g., in the respective sample). In some embodiments, the respective (e.g., second or third) plurality of sequence reads exhibits an average read depth of at least 10, at least 20, at least 40, at least 50, at least 100, at least 200, or at least 300 across the respective subset of the genome of the subject. In some embodiments, the respective (e.g., second or third) plurality of sequence reads exhibits an average read depth of no more than 500, no more than 300, no more than 200, no more than 100, no more than 50, no more than 40, or no more than 20 across the respective subset of the genome of the subject. In some embodiments, the plurality of sequence reads exhibits an average read depth of 10 to 40, from 25 to 60, from 30 to 100, or from 100 to 500 across the respective subset of the genome of the subject. In some embodiments, the plurality of sequence reads exhibits an average read depth that falls within another range starting no lower than 10 and ending no higher than 500 across the respective subset of the genome of the subject.

In some embodiments, a respective (e.g., second or third) plurality of sequence reads comprises whole genome sequencing reads. In some embodiments, a respective plurality of sequence reads comprises exome sequence reads. In some embodiments, a respective plurality of sequence reads comprises targeted sequencing reads.

In some embodiments, a respective subset of the genome is at least 1, at least 10, at least 20, at least 30, at least 40, at least 50, at least 70, at least 80, at least 90, at least 95, or at least 99 percent of a respective single chromosome of the subject. In some embodiments, a respective subset of the genome is no more than 100, no more than 99, no more than 95, no more than 90, no more than 80, no more than 50, no more than 30, or no more than 10 percent of a respective single chromosome of the subject. In some embodiments, a respective subset of the genome is between 1 and 10 percent, between 5 and 15 percent, between 10 and 20 percent, between 15 and 30 percent, between 25 and 50 percent, between 45 and 75 percent, or between 70 and 100 percent of a respective single chromosome of the subject. In some embodiments, a respective subset of the genome falls within another range starting no lower than 1 percent and ending no higher than 100 percent of a respective single chromosome of the subject.

In some embodiments, a respective subset of the genome is at least 1, at least 10, at least 20, at least 30, at least 40, at least 50, at least 70, at least 80, at least 90, at least 95, or at least 99 percent of a respective two or more chromosomes of the subject. In some embodiments, a respective subset of the genome is no more than 100, no more than 99, no more than 95, no more than 90, no more than 80, no more than 50, no more than 30, or no more than 10 percent of a respective two or more chromosomes of the subject. In some embodiments, a respective subset of the genome is between 1 and 10 percent, between 5 and 15 percent, between 10 and 20 percent, between 15 and 30 percent, between 25 and 50 percent, between 45 and 75 percent, or between 70 and 100 percent of a respective two or more chromosomes of the subject. In some embodiments, a respective subset of the genome falls within another range starting no lower than 1 percent and ending no higher than 100 percent of a respective two or more chromosomes of the subject.

In some embodiments, a respective two or more chromosomes of the subject includes at least 2, at least 3, at least 4, at least 5, at least 10, at least 15, at least 20, at least 30, or at least 40 chromosomes. In some embodiments, a respective two or more chromosomes of the subject includes no more than 46, no more than 40, no more than 30, no more than 20, no more than 10, or no more than 5 chromosomes. In some embodiments, a respective two or more chromosomes of the subject consists of from 2 to 10, from 5 to 20, from 18 to 40, or from 30 to 46 chromosomes. In some embodiments, a respective two or more chromosomes of the subject falls within another range starting no lower than 2 chromosomes and ending no higher than 46 chromosomes.

In some embodiments, a respective subset of the genome is at least 1, at least 10, at least 20, at least 30, at least 40, at least 50, at least 70, at least 80, at least 90, at least 95, or at least 99 percent of the genome of the subject. In some embodiments, a respective subset of the genome is no more than 100, no more than 99, no more than 95, no more than 90, no more than 80, no more than 50, no more than 30, or no more than 10 percent of the genome of the subject. In some embodiments, a respective subset of the genome is between 1 and 10 percent, between 5 and 15 percent, between 10 and 20 percent, between 15 and 30 percent, between 25 and 50 percent, between 45 and 75 percent, or between 70 and 100 percent of the genome of the subject. In some embodiments, a respective subset of the genome falls within another range starting no lower than 1 percent and ending no higher than 100 percent of the genome of the subject.

In some embodiments, a respective subset of the genome comprises a respective exome of the subject. In some embodiments, a respective subset of the genome consists of all or a portion of a respective exome of the subject.

Referring to block 314, in some embodiments, the method further includes performing a first exome sequencing with at least 100× coverage to obtain the second plurality of sequence reads; and performing a second exome sequencing with at least 100× coverage to obtain the second plurality of sequence reads.

Accordingly, in some embodiments, a respective (e.g., second or third) plurality of sequence reads encompasses a respective subset of the exome of the subject (e.g., in a respective sample) and not the rest of the exome of the subject (e.g., in the respective sample). In some embodiments, the respective (e.g., second or third) plurality of sequence reads exhibits a coverage of at least 10×, at least 20×, at least 40×, at least 50×, at least 100×, at least 200×, at least 300×, at least 500×, or at least 1000× across the respective subset of the exome of the subject. In some embodiments, the respective (e.g., second or third) plurality of sequence reads exhibits a coverage of no more than 2000×, no more than 1000×, no more than 500×, no more than 300×, no more than 200×, no more than 100×, no more than 50×, no more than 40×, or no more than 20× across the respective subset of the exome of the subject. In some embodiments, the plurality of sequence reads exhibits a coverage of 10× to 100×, from 50× to 400×, from 200× to 1000×, or from 600× to 2000× across the respective subset of the exome of the subject. In some embodiments, the plurality of sequence reads exhibits a coverage that falls within another range starting no lower than 10× and ending no higher than 2000× across the respective subset of the exome of the subject.

In some embodiments, a respective subset of the exome is at least 1, at least 10, at least 20, at least 30, at least 40, at least 50, at least 70, at least 80, at least 90, at least 95, or at least 99 percent of the exome of the subject. In some embodiments, a respective subset of the exome is no more than 100, no more than 99, no more than 95, no more than 90, no more than 80, no more than 50, no more than 30, or no more than 10 percent of the exome of the subject. In some embodiments, a respective subset of the exome is between 1 and 10 percent, between 5 and 15 percent, between 10 and 20 percent, between 15 and 30 percent, between 25 and 50 percent, between 45 and 75 percent, or between 70 and 100 percent of the exome of the subject. In some embodiments, a respective subset of the exome falls within another range starting no lower than 1 percent and ending no higher than 100 percent of the exome of the subject.

In some embodiments, the plurality of somatic variants (e.g., obtained using the second plurality of sequence reads from DNA molecules in the tumor sample and the third plurality of sequence reads from DNA molecules in a normal sample) is further validated using the first plurality of sequence reads obtained from RNA molecules in the tumor sample, where the validation retains in the first plurality of somatic variants those somatic variants observed in the first plurality of sequence reads. In some embodiments, the validation removes from the first plurality of somatic variants those somatic variants that are not also observed in the first plurality of sequence reads obtained from RNA molecules in the tumor sample. Accordingly, in some such embodiments, the method further includes using RNA from the tumor sample to validate the presence of somatic variants determined using DNA in the tumor sample and/or the normal sample.

As an illustrative example, in some implementations, the plurality of somatic variants comprises one or more indels, where each respective indel in the one or more indels is validated using the first plurality of sequence reads obtained from RNA molecules in the tumor sample. In some such embodiments, the validation retains in the first plurality of somatic variants those indels that are also observed in the first plurality of sequence reads (e.g., RNA) from the tumor sample. In some embodiments, the validation removes from the first plurality of somatic variants those indels that are not also observed in the first plurality of sequence reads (e.g., RNA) from the tumor sample.

### Consideration of Normal Sample.

Referring to block 316, in some embodiments, the normal sample is a tissue sample proximate to an original location of the tumor in the subject.

In some embodiments, the present disclosure provides for the identification of a tumor vaccine personalized to a human subject afflicted with a cancer where adjacent normal tissue that is proximate to a location of a tumor in the subject is considered. For instance, in some embodiments, the method includes obtaining nucleic acid molecules from a sample of a tumor and nucleic acid molecules from a normal sample that is proximate to an original location of the tumor in the subject, where (i) the nucleic acid molecules from the sample of the tumor include at least DNA from the sample of the tumor and (ii) the nucleic acid molecules from the normal sample include at least DNA from the normal sample. In some embodiments, the nucleic acid molecules further include RNA from the sample of the tumor and/or RNA from the normal sample. In some embodiments, the DNA and/or RNA from the sample of the tumor and/or the normal sample are sequenced to obtain corresponding one or more pluralities of sequence reads.

As described above, in some embodiments, the first plurality of somatic variants is determined by including in the first plurality of somatic variants those somatic variants observed in a second plurality of sequence reads (e.g., DNA) from the tumor sample that are not observed in a third plurality of sequence reads (e.g., DNA) from the normal sample. In some embodiments, the plurality of somatic variants is further validated using the first plurality of sequence reads obtained from RNA molecules in the tumor sample and a fourth plurality of sequence reads obtained from RNA molecules in the normal sample, where the validation retains in the first plurality of somatic variants those somatic variants observed in the first plurality of sequence reads (e.g., RNA) from the tumor sample that are not observed in the fourth plurality of sequence reads (e.g., RNA) from the normal sample. In some embodiments, the validation removes from the first plurality of somatic variants those somatic variants observed in the first plurality of sequence reads (e.g., RNA) from the tumor sample that are also observed in the fourth plurality of sequence reads (e.g., RNA) from the normal sample.

As an illustrative example, in some implementations, the plurality of somatic variants comprises one or more indels, where each respective indel in the one or more indels is validated using the first plurality of sequence reads obtained from RNA molecules in the tumor sample and the fourth plurality of sequence reads obtained from RNA molecules in the normal sample. In some such embodiments, the validation retains in the first plurality of somatic variants those indels observed in the first plurality of sequence reads (e.g., RNA) from the tumor sample that are not observed in the fourth plurality of sequence reads (e.g., RNA) from the normal sample. In some embodiments, the validation removes from the first plurality of somatic variants those indels observed in the first plurality of sequence reads (e.g., RNA) from the tumor sample that are also observed in the fourth plurality of sequence reads (e.g., RNA) from the normal sample.

Moreover, in some embodiments, this approach can be extended to determine and/or validate one or more fusion proteins that are present in the tumor sample that are not observed in the normal sample. For instance, in some embodiments, one or more fusion proteins is determined by including in the one or more fusion proteins those fusion proteins observed the first plurality of sequence reads (e.g., RNA) from the tumor sample that are not observed in the fourth plurality of sequence reads (e.g., RNA) from the normal sample. In some embodiments, the validation removes from the one or more fusion proteins those fusion proteins observed in the first plurality of sequence reads (e.g., RNA) from the tumor sample that are also observed in the fourth plurality of sequence reads (e.g., RNA) from the normal sample.

In contrast to conventional approaches that consider only normal blood and tumor tissue, in some embodiments, and without being limited to any one theory of operation, incorporating adjacent normal tissue advantageously enables more accurate identification of bona fide cancer mutations, as observed mutations (e.g., candidate neoantigens) with evidence in healthy prostate tissue can be discarded as potentially indicating sequencing errors, germline mutations, or non-tumor somatic mosaicism. Accordingly, the use of adjacent normal tissue in addition to tumor samples, in some embodiments, meaningfully improve identification of candidate neoantigens for tumor vaccines, for example, through comparative somatic variant and/or fusion protein identification.

Referring to block 318, in some embodiments, the normal sample is a blood sample. In some embodiments, the normal sample is a tissue sample.

In certain embodiments, the normal sample is a biological tissue or fluid. Non-limiting biological samples include bone marrow, blood, blood cells, ascites, (tissue or fine needle) biopsy samples, cell-containing body fluids, free floating nucleic acids, sputum, saliva, urine, cerebrospinal fluid, peritoneal fluid, pleural fluid, feces, lymph, gynecological fluids, swabs (e.g., skin swabs, vaginal swabs, oral swabs, and nasal swabs), washings or lavages such as a ductal lavages or broncheoalveolar lavages, aspirates, scrapings, specimens (e.g., bone marrow specimens, tissue biopsy specimens, and surgical specimens), feces, other body fluids, secretions, and/or excretions, and cells therefrom.

In some embodiments, the normal sample is obtained using any suitable method for obtaining biological samples known in the art, as described above with reference to obtaining tumor samples. In some implementations, the normal sample is a tissue biopsy sample. In some embodiments, the normal sample is a formalin-fixed tissue (FFT), such as a formalin-fixed paraffin-embedded (FFPE) tissue. In some embodiments, the normal sample is an FFPE or FFT block. In some embodiments, the normal sample is a cryo-section of a tissue biopsy and/or a core needle biopsy. In some embodiments, the normal sample is a fresh frozen sample. In some embodiments, the normal sample is OCT-embedded.

In some embodiments, the determining the first plurality of somatic variants of the subject includes performing, for each respective sequence read in the second plurality of sequence reads from DNA molecules in the tumor sample obtained from the subject, and for each respective sequence read in the third plurality of sequence reads from DNA molecules in the normal sample obtained from the subject, a sequence alignment procedure and/or a mutation identification (e.g., variant calling) procedure.

In some implementations, sequence alignment is performed by aligning sequence reads to a reference human genome (e.g., hg19) using an alignment tool such as the Burrows-Wheeler Alignment tool. In some embodiments, base-quality score recalibration and/or duplicate-read removal is performed. In some embodiments, the recalibration excludes germline variants, annotation of mutations, and indels as described in Snyder et al, 2014, “Genetic Basis for Clinical Response to CTLA-4. Blockade in Melanoma,” N. Engl. J. Med. 371, 2189-2199, which is hereby incorporated by reference. In some embodiments, the local realignment and quality score recalibration are conducted using the Genome Analysis Toolkit (GATK) according to GATK best practices. See, DePristo et al., 2011, “A framework for variation discovery and genotyping using next-generation DNA sequencing data,” Nature Genet. 43, pp. 491-498; and Van der Auwera et al, 2013, “From FastQ Data to High-Confidence Variant Calls: The Genome Analysis Toolkit Best Practices Pipeline,” Curr. Prot. in Bioinformatics 43, 11.10.1-11.10.33 each of which is hereby incorporated by reference. In some embodiments, sequence alignment and mutation identification are performed using FASTQ files that are processed to remove any adapter sequences at the end of the reads. In some embodiments, adapter sequences are removed using cutadapt (v1.6). See, Martin, 2011, “Cutadapt removes adapter sequences from high-throughput sequencing reads,” EMBnet.journal 17, pp. 10-12, which is hereby incorporated by reference. Then, resulting files are mapped using a mapping software such as the BWA mapper (bwa mem v0.7.12), (see, e.g., Li and Durbin, 2009, “Fast and accurate short read alignment with Burrows-Wheeler Transform,” Bioinformatics 25, pp. 1754-1760, which is hereby incorporated by reference). In some such embodiments, the resulting files (e.g., SAM files) are sorted and read group tags are added using the PICARD tools. After sorting in coordinate order, the BAMs are processed with a tool such as PICARD MarkDuplicates. In some embodiments, realignment and recalibration are then conducted (e.g., with a first realignment using the InDel realigner followed by base quality value recalibration with the BaseQRecalibrator). Once realignment and recalibration have been performed, mutation callers are then used to identify somatic variants. Methods and tools for mutation identification (e.g., variant calling) are known in the art. For instance, exemplary suitable mutation callers contemplated for use in the present disclosure include, but are not limited to, Mutect, Somatic Sniper, Varscan, VarDict, FastD, and/or Strelka. See, e.g., Wei et al, 2015, “MAC: identifying and correcting annotation for multi-nucleotide variations,” BMC Genomics 16, p. 569; Snyder and Chan, 2015, “Immunogenic peptide discovery in cancer genomes,” Curr Opin Genet Dev 30, pp. 7-16; Nielsen et al., 2003, “Reliable prediction of T-cell epitopes using neural networks with novel sequence representations,” Protein Sci 12, pp. 1007-1017; Shen and Seshan, 2016, “FACETS: allele-specific copy number and clonal heterogeneity analysis tool for high-throughput DNA sequencing,” Nucleic Acids Res. 44, e131; and Roudko et al., “Computational prediction and validation of tumor-associated neoantigens,” Front Immunol. 2020; 11:27, each of which is incorporated by reference.

### Determining Fusion Proteins.

As described above, referring again to block 320, the method includes obtaining a first plurality of sequence reads 216 from RNA molecules in a sample of a tumor obtained from the subject.

Referring to block 326, the method further includes determining, from the first plurality of sequence reads 216, one or more fusion proteins 218 encoded by the first plurality of sequence reads 216, where each respective fusion protein 218 in the one or more fusion proteins is a fusion of a portion of a respective first human protein 220-1 and a portion of a respective second human protein 220-2.

In some embodiments, the present disclosure provides systems and methods for identifying tumor vaccines for vaccination of a subject against neoantigens derived from fusion protein products. For instance, in some settings such as prostate cancer where gene fusions are common, a vaccine that incorporates neoantigens derived from fusion proteins is desired. Existing conventional pipelines, however, generally include only small coding mutations such as missense and frameshift mutations. Advantageously, the present disclosure provides an approach for vaccinating with neoantigens that are predicted to arise from gene fusions. As an illustrative example, in some implementations, the approach utilizes a consensus approach that integrates one or more methods for fusion protein identification (e.g., fusion callers such as STAR-Fusion, FusionCatcher, Arriba, and/or FusionInspector) to identify high-confidence fusions. See, for instance, Example 3 below. The fusion protein sequence is then predicted using one or more methods for fusion protein sequence prediction (e.g., prediction tools such as STAR-Fusion and/or Fusion-Inspector). In some implementations, the present disclosure further provides systems and methods for predicting candidate neoantigens that span the fusion boundary (e.g., candidate neoantigens that encode one or more residues from a portion of a respective first human protein and one or more residues from a portion of a respective second human protein). These approaches allow for the identification of tumor vaccines that incorporate fusion protein products as vaccination targets, which are likely to be beneficial over traditional approaches for the treatment of cancers where gene fusions are common. Non-limiting example fusion proteins found in cancers such as prostate cancer are described, for instance, in Examples 2-4 below. Additional predicted fusion protein sequences capable of inducing an immune response in T cells are also disclosed, for example, in Example 7 below.

In some embodiments, the one or more fusion proteins includes at least 2, at least 5, at least 10, at least 20, at least 50, at least 100, at least 200, at least 500, or at least 800 fusion proteins. In some embodiments, the one or more fusion proteins includes no more than 1000, no more than 800, no more than 500, no more than 200, no more than 100, no more than 50, no more than 20, no more than 10, or no more than 5 fusion proteins. In some embodiments, the one or more fusion proteins consists of from 2 to 10, from 2 to 50, from 5 to 80, from 50 to 200, from 100 to 500, or from 300 to 1000 fusion proteins. In some embodiments, the one or more fusion proteins falls within another range starting no lower than 2 fusion proteins and ending no higher than 1000 fusion proteins.

In some implementations, and without being limited by any one theory of operation, fusion genes represent an important class of genomic alteration contributing to the tumorigenesis for both solid and hematological cancers. These hybrid genes are often produced by recurrent chromosomal rearrangements, such as translocation, deletion, and insertion. In some embodiments, the one or more fusion proteins are determined using a fusion protein detector. In some embodiments, the fusion protein detector utilizes breakpoint prediction to identify a fusion junction (e.g., where a portion of a respective first human protein and a portion of a respective second human protein combine). Fusion protein detectors are known in the art. Non-limiting examples of tools for fusion protein detection contemplated for use in the present disclosure include STAR-Fusion, FusionCatcher, Arriba, FusionInspector, TopHat-Fusion, JAFFA, Fuseq, SvABA, LUMPY, GRIDSS, and/or SVcaller. See, e.g., Deng et al., “Fusion gene detection using whole-exome sequencing data in cancer patients,” Front Genet. 2022; 13, which is hereby incorporated herein by reference in its entirety.

In some embodiments, the one or more fusion proteins are determined using a plurality of fusion protein detectors. In some such embodiments, for each respective fusion protein detector in the plurality of fusion protein detectors, a corresponding set of candidate fusion proteins are obtained, thereby obtaining a plurality of candidate fusion proteins. In some embodiments, the determining further includes selecting, from the plurality of candidate fusion proteins, one or more candidate fusion proteins that are shared between at least two sets of candidate fusion proteins in the plurality of sets of candidate fusion proteins. In other words, in some embodiments, the one or more fusion proteins are determined by selecting candidate fusion proteins that are predicted by at least two fusion protein detectors in the plurality of fusion protein detectors.

In some embodiments, the plurality of fusion protein detectors includes at least 2, at least 3, at least 4, at least 5, or at least 6 fusion protein detectors. In some embodiments, the plurality of fusion protein detectors includes no more than 10, no more than 6, no more than 4, or no more than 3 fusion protein detectors. In some embodiments, the plurality of fusion protein detectors consists of from 2 to 5, from 3 to 8, or from 5 to 10 fusion protein detectors. In some embodiments, the plurality of fusion protein detectors falls within another range starting no lower than 2 fusion protein detectors and ending no higher than 10 fusion protein detectors.

In some embodiments, the one or more fusion proteins are determined by selecting candidate fusion proteins that are predicted by at least 2, at least 3, at least 4, at least 5, or at least 6 fusion protein detectors in the plurality of fusion protein detectors. In some embodiments, the one or more fusion proteins are determined by selecting candidate fusion proteins that are predicted by no more than 10, no more than 6, no more than 4, or no more than 3 fusion protein detectors in the plurality of fusion protein detectors. In some embodiments, the one or more fusion proteins are determined by selecting candidate fusion proteins that are predicted by from 2 to 5, from 3 to 8, or from 5 to 10 fusion protein detectors in the plurality of fusion protein detectors. In some embodiments, the one or more fusion proteins are determined by selecting candidate fusion proteins that are predicted another range of fusion protein detectors in the plurality of fusion protein detectors starting no lower than 2 fusion protein detectors and ending no higher than 10 fusion protein detectors.

In some embodiments, as described above, the one or more fusion proteins is further validated using the first plurality of sequence reads obtained from RNA molecules in the tumor sample and a fourth plurality of sequence reads obtained from RNA molecules in the normal sample, where the validation retains in the one or more fusion proteins those fusion observed in the first plurality of sequence reads (e.g., RNA) from the tumor sample that are not observed in the fourth plurality of sequence reads (e.g., RNA) from the normal sample. In some embodiments, the validation removes from the one or more fusion proteins those fusion proteins observed in the first plurality of sequence reads (e.g., RNA) from the tumor sample that are also observed in the fourth plurality of sequence reads (e.g., RNA) from the normal sample.

In some embodiments, the method further includes validating the one or more fusion proteins in the first, second, third, and/or fourth plurality of sequence reads, using polymerase chain reaction and/or one or more additional sequencing steps (e.g., Sanger sequencing).

In some embodiments, the normal sample is a tissue sample proximate to an original location of the tumor in the subject. In some embodiments, the normal sample is a blood sample. In some embodiments, the normal sample is a tissue sample.

In some embodiments, the cancer is prostate cancer, and the one or more fusion proteins are selected from the group consisting of: KANSL1-ARL17A, TMPRSS2-ERG, EIF3B-FOXK2, SLC45A3-BRAF, and/or ESRP1-RAF1. In some embodiments, the cancer is prostate cancer, and the one or more fusion proteins are selected from the group consisting of TMPRSS2 fused to an ETS family transcription factor (e.g., ETV1, ETV4, etc.).

### Selecting Candidate Neoantigens.

Referring to block 328, the method further includes selecting a plurality of candidate neoantigens 232 comprising a first subset of candidate neoantigens 234-1 and a second subset of candidate neoantigens 234-2, where each neoantigen 232 in the first subset of candidate neoantigens 234-1 encodes a somatic variant 214 in the first plurality of somatic variants, and each neoantigen 232 in the second subset of candidate neoantigens 234-2 encodes one or more residues from the portion of the respective first human protein 220-1 and one or more residues from the portion of the respective second human protein 220-2.

In other words, in some embodiments, each respective neoantigen in the second subset of candidate neoantigens encodes, for a respective fusion protein in the one or more fusion proteins, a corresponding one or more residues from the portion of the respective first human protein and a corresponding one or more residues from the portion of the respective second human protein of the respective fusion protein.

In some embodiments, the selecting the plurality of candidate neoantigens comprises determining a protein sequence of one or more somatic variants in the first plurality of somatic variants.

In some embodiments, the selecting the plurality of candidate neoantigens comprises determining a protein sequence of a respective fusion protein in the one or more fusion proteins.

Tools and methods for determining protein sequences, including somatic variant sequences and fusion protein sequences, are known in the art. For instance, example tools suitable for determining protein sequences include, but are not limited to, STAR-Fusion, Fusion-Inspector, Vaxrank, Isovar, Varcode, PyEnsembl, and/or MHCtools. Vaxrank is an overall vaccine selection tool with ranking logic. Isovar determines mutant protein sequence from somatic variants and tumor RNA. Varcode predicts variant effects for filtering out silent mutations. PyEnsembl provides reference genome annotations that are used by Varcode to determine exon boundaries and transcript sequences. MHCtools is a common interface to peptide-MHC-binding predictors. See, e.g., Rubinsteyn et al., “Computational pipeline for the PGV-001 neoantigen vaccine trial,” Front Immunol. 2018; 8, which is hereby incorporated herein by reference in its entirety.

In some embodiments, the selecting the plurality of candidate neoantigens comprises obtaining, for each respective somatic variant in the first plurality of somatic variants, a respective neoantigen that corresponds to the respective somatic variant. In some embodiments, the respective neoantigen has a protein sequence corresponding to all or a portion of a nucleic acid sequence of the respective somatic variant.

In some embodiments, the first subset of candidate neoantigens (e.g., encoding somatic variants in the first plurality of somatic variants) comprises candidate neoantigens that collectively represent all or a portion of the first plurality of somatic variants. In some embodiments, the first subset of candidate neoantigens collectively represents at least 10, at least 20, at least 30, at least 40, at least 50, at least 60, at least 70, at least 80, at least 90, at least 95, or at least 99 percent of the first plurality of somatic variants. In some embodiments, the first subset of candidate neoantigens collectively represents no more than 100, no more than 99, no more than 95, no more than 90, no more than 80, no more than 50, no more than 30, or no more than 20 percent of the first plurality of somatic variants. In some embodiments, the first subset of candidate neoantigens collectively represents from 10 to 40, from 30 to 60, from 40 to 90, from 50 to 95, from 70 to 99, or from 80 to 100 percent of the first plurality of somatic variants. In some embodiments, the first subset of candidate neoantigens collectively represents another range of the first plurality of somatic variants starting no lower than 10 percent and ending no higher than 100 percent.

In some embodiments, the selecting the plurality of candidate neoantigens comprises obtaining, for each respective fusion protein in the one or more fusion proteins, a respective neoantigen that corresponds to the respective fusion protein. In some embodiments, the respective neoantigen has a protein sequence corresponding to all or a portion of a nucleic acid sequence of the respective fusion protein.

In some embodiments, the second subset of candidate neoantigens (e.g., encoding one or more fusion proteins) comprises candidate neoantigens that collectively represent all or a portion of the one or more fusion proteins. In some embodiments, the second subset of candidate neoantigens collectively represents at least 10, at least 20, at least 30, at least 40, at least 50, at least 60, at least 70, at least 80, at least 90, at least 95, or at least 99 percent of the one or more fusion proteins. In some embodiments, the second subset of candidate neoantigens collectively represents no more than 100, no more than 99, no more than 95, no more than 90, no more than 80, no more than 50, no more than 30, or no more than 20 percent of the one or more fusion proteins. In some embodiments, the second subset of candidate neoantigens collectively represents from 10 to 40, from 30 to 60, from 40 to 90, from 50 to 95, from 70 to 99, or from 80 to 100 percent of the one or more fusion proteins. In some embodiments, the second subset of candidate neoantigens collectively represents another range of the one or more fusion proteins starting no lower than 10 percent and ending no higher than 100 percent.

Referring to block 330, in some embodiments, the plurality of candidate neoantigens comprises 5 candidate neoantigens.

In some embodiments, the plurality of candidate neoantigens includes at least 2, at least 5, at least 10, at least 20, at least 50, at least 100, at least 200, at least 500, or at least 1000 candidate neoantigens. In some embodiments, the plurality of candidate neoantigens includes no more than 5000, no more than 1000, no more than 500, no more than 200, no more than 100, no more than 50, no more than 20, no more than 10, or no more than 5 candidate neoantigens. In some embodiments, the plurality of candidate neoantigens consists of from 2 to 10, from 2 to 50, from 5 to 80, from 50 to 200, from 100 to 500, from 300 to 2000, or from 1000 to 5000 candidate neoantigens. In some embodiments, the plurality of candidate neoantigens falls within another range starting no lower than 2 candidate neoantigens and ending no higher than 5000 candidate neoantigens.

In some embodiments, the first subset of candidate neoantigens includes at least 2, at least 5, at least 10, at least 20, at least 50, at least 100, at least 200, at least 500, or at least 800 candidate neoantigens. In some embodiments, the first subset of candidate neoantigens includes no more than 1000, no more than 800, no more than 500, no more than 200, no more than 100, no more than 50, no more than 20, no more than 10, or no more than 5 candidate neoantigens. In some embodiments, the first subset of candidate neoantigens consists of from 2 to 10, from 2 to 50, from 5 to 80, from 50 to 200, from 100 to 500, or from 300 to 1000 candidate neoantigens. In some embodiments, the first subset of candidate neoantigens falls within another range starting no lower than 2 candidate neoantigens and ending no higher than 1000 candidate neoantigens.

In some embodiments, the second subset of candidate neoantigens includes at least 2, at least 5, at least 10, at least 20, at least 50, at least 100, at least 200, at least 500, or at least 800 candidate neoantigens. In some embodiments, the second subset of candidate neoantigens includes no more than 1000, no more than 800, no more than 500, no more than 200, no more than 100, no more than 50, no more than 20, no more than 10, or no more than 5 candidate neoantigens. In some embodiments, the second subset of candidate neoantigens consists of from 2 to 10, from 2 to 50, from 5 to 80, from 50 to 200, from 100 to 500, or from 300 to 1000 candidate neoantigens. In some embodiments, the second subset of candidate neoantigens falls within another range starting no lower than 2 candidate neoantigens and ending no higher than 1000 candidate neoantigens.

Referring to block 332, in some embodiments, each respective candidate neoantigen in the plurality of candidate neoantigens has a length of N residues, wherein N is 8, 9, 10, 11, 12, 13, or 14.

In some embodiments, N is at least 4, at least 6, at least 8, at least 10, at least 15, at least 20, or at least 30. In some embodiments, N is no more than 50, no more than 30, no more than 20, no more than 15, no more than 10, or no more than 5. In some embodiments, N is from 4 to 15, from 8 to 20, from 8 to 14, from 15 to 30, or from 20 to 50. In some embodiments, N falls within another range starting no lower than 4 and ending no higher than 50.

Referring to block 334, in some embodiments, each respective candidate neoantigen in the plurality of candidate neoantigens has a length of 9 residues.

Referring to block 336, in some embodiments, the method further includes using the first plurality of sequence reads to validate a plurality of indel mutations present in the tumor sample. In some such embodiments, the plurality of candidate neoantigens comprises a third subset of candidate neoantigens, and each neoantigen in the third subset of the plurality of candidate neoantigens encodes all or a portion of an indel mutation in the plurality of indel mutations.

For instance, as described above, in some embodiments, the method further includes using RNA (e.g., from the tumor sample and/or a normal sample) to validate the presence of somatic variants (e.g., indels) determined using DNA (e.g., in the tumor sample and/or the normal sample).

In some embodiments, the third subset of candidate neoantigens includes at least 2, at least 5, at least 10, at least 20, at least 50, at least 100, at least 200, at least 500, or at least 800 candidate neoantigens. In some embodiments, the third subset of candidate neoantigens includes no more than 1000, no more than 800, no more than 500, no more than 200, no more than 100, no more than 50, no more than 20, no more than 10, or no more than 5 candidate neoantigens. In some embodiments, the third subset of candidate neoantigens consists of from 2 to 10, from 2 to 50, from 5 to 80, from 50 to 200, from 100 to 500, or from 300 to 1000 candidate neoantigens. In some embodiments, the third subset of candidate neoantigens falls within another range starting no lower than 2 candidate neoantigens and ending no higher than 1000 candidate neoantigens.

### Scoring by Class I MHC Affinity.

Referring to block 338, the method further includes determining a respective score 242 for each respective candidate neoantigen 234 in the plurality of candidate neoantigens using a scoring function that includes a first scoring term that upweights respective candidate neoantigens 234 having a higher class I major histocompatibility complex (MHC) affinity relative to respective candidate neoantigens having a lower class I MHC affinity, given a class I human leukocyte antigen (HLA) type of the human subject.

In some embodiments, the first scoring term is determined as an amplitude A of the respective candidate neoantigen, where the amplitude A of the respective neoantigen is computed as a function of the relative major histocompatibility complex (MHC) affinity between a respective mutant peptide (e.g., a candidate neoantigen) and its wildtype counterpart given the HLA type of the subject. In other words, the amplitude, A, is the ratio of the relative probability that a candidate neoantigen is bound on class I MHC times the relative probability that a candidate neoantigen's wildtype counterpart is not bound. In some embodiments, the amplitude

$$A = ( {P}_{U}^{WT} / {P}_{B}^{WT} ) \times ( {P}_{B}^{MT} / {P}_{U}^{MT} ) ,$$

where

$${P}_{B}^{MT}$$

is the binding probability of a candidate neoantigen,

$${P}_{B}^{WT}$$

is the binding probability of its wildtype counterpart, and

$${P}_{U}^{WT} = 1 - {P}_{B}^{WT} and {P}_{U}^{MT} = 1 - {P}_{B}^{MT} .$$

As a result, the amplitude, A, rewards cases where the discrimination energy between a mutant and wildtype peptide by the same class I MHC molecule (e.g., the same HLA allele) is large (see, e.g., Storma, 2013, Quantitative Biol. 1, p 115), while the mutant binding energy is kept low. The τ parameter effectively sets this energy scale for dominant neoantigens in a cline when R=1. Assuming similar concentrations for mutant and wildtype peptides, the amplitude is the ratio of wildtype to mutant dissociation constants:

$$A = {K}_{d}^{WT} / {K}_{d}^{MT} .$$

Generally, negative thymic selection on TCRs is not absolute, but rather “prunes” the repertoire recognizing the self-proteome (see, e.g., Yu et al., 2015, Immunity 42, p. 929; and Legoux et al., 2015, Immunity 43, p. 896). The amplitude A is therefore used, in some implementations, as a proxy for the availability of TCRs in the repertoire to recognize a candidate neoantigen. In some instances, candidate neoantigens differ from their wildtype peptides by only a single mutation. Given the uniqueness of nonamer sequence in the self-proteome due to finite genome size (SI), it is highly improbable that the mutant peptide would have another 8-mer match in the human proteome, such that, in some embodiments, only the comparison with the respective wildtype peptide is considered. In some instances, the amplitude can be interpreted as a multiplicity of receptors available to cross-reactively recognize a neoantigen.

In some embodiments, the MHC presentation is quantified, as amplitude A, using the relative MHC affinity between the wildtype peptide and mutant candidate neoantigen, a ratio used to analyze computational neoantigen predictions. See, Hundal et al., 2016, “pVAC-Seq: A genome-guided in silico approach to identifying tumor neoantigens,” Genome Med. 8, 1-11, which is hereby incorporated herein by reference in its entirety. The relative MHC affinity rewards mutant neoantigens with strong mutant affinities compared to wildtype. Without intending to be limited to any particular theory, it is posited that the wildtype peptides presented by MHC are potentially subject to tolerance and hence, due to homology, their mutant counterparts may be as well, compromising their immunogenicity.

In some embodiments, the function of the relative class I MHC affinity of the respective neoantigen and the wildtype counterpart of the respective candidate neoantigen given the HLA type of the subject is a ratio of: (1) a dissociation constant between the respective candidate neoantigen and the class I MHC presented by the cancer subject given the HLA type of the cancer subject, and (2) a dissociation constant between the wildtype counterpart of the respective candidate neoantigen and the class I MHC presented by the cancer subject given the HLA type of the cancer subject.

In some such embodiments, the dissociation constant between the respective candidate neoantigen and the class I MHC presented by the cancer subject is obtained as output from a first classifier upon inputting into the first classifier the amino acid sequence of the candidate neoantigen. Thus, the dissociation constant between the wildtype counterpart of the respective candidate neoantigen and the class I MHC presented by the cancer subject of the HLA type of the subject is obtained as output from the first classifier upon inputting into the first classifier the amino acid sequence of the respective wildtype counterpart of the candidate neoantigen (e.g., the first classifier is specific to the HLA type of the cancer subject and has been trained with the respective class I MHC binding coefficient and sequence data of each peptide epitope in a plurality of epitopes presented by class I MHC in a training population having the HLA type of the subject).

Suitable non-limiting methods for determining class I MHC affinity of candidate neoantigens, given a class I human leukocyte antigen (HLA) type of the human subject, are further disclosed in, for example, PCT Application No. PCT/US2018/014282, filed Jan. 18, 2018, entitled “Neoantigens and uses thereof for treating cancer,” which is hereby incorporated herein by reference in its entirety.

In some embodiments, the first scoring term is obtained by a method comprising obtaining a class I MHC affinity for each respective candidate neoantigen in the plurality of candidate neoantigens in silico using computational methods to predict peptide binding-affinity to HLA molecules. In some embodiments, MHC affinity prediction is based on artificial neural networks with predicted IC₅₀. For example, in some implementations, the NetMHCpan or NetMHC software is used to predict peptide binding to alleles for which no ligands have been reported. See, for example, Roudko et al., “Computational prediction and validation of tumor-associated neoantigens,” Front Immunol. 2020; 11:27, which is hereby incorporated herein by reference in its entirety. Suitable non-limiting methods for determining class I MHC affinity of candidate neoantigens, given a class I human leukocyte antigen (HLA) type of the human subject, further include any of the methods for determining class II MHC affinity of candidate neoantigens, as described elsewhere herein (see, for example, the section entitled “Additional scoring terms,” below).

Referring to block 340, in some embodiments, the method further includes determining the class I HLA type of the human subject using the first plurality of sequencing reads.

Generally, CD8+ T cells recognize antigens presented on the MHC-I complex, which is composed of conserved b2-microglobulin and a variable a-chain. The latter subunit is highly polymorphic and encoded within the HLA gene, which is represented by three loci on human chromosome 6: HLA-A, HLA-B, and HLA-C. Thus, HLA allele assignment consists of the gene name (A, B, or C) followed by a set of digits separated by colons, where the first two digits specify serological activity (A*01, B*03, etc.) and the second two digits indicate protein sequence (A*01:05, B*03:05, etc.). Typically, the HLA gene exhibits a high of polymorphism, such that precise HLA-allele typing at protein level resolution from WES and RNA-seq reads can be a complex task. See, for example, Roudko et al., “Computational prediction and validation of tumor-associated neoantigens,” Front Immunol. 2020; 11:27, which is hereby incorporated herein by reference in its entirety.

Methods for determining class I HLA type are known in the art. For example, several tools have been developed to obtain HLA allele information from genome-wide sequencing data (e.g., whole-exome, whole-genome, and RNA sequencing data), including OptiType, Polysolver, PHLAT, HLAreporter, HLAforest, HLAminer, and seq2HLA (see, for example, Kiyotani K et al., “Immunopharmacogenomics towards personalized cancer immunotherapy targeting neoantigens,” Cancer Science 2018; 109:542-549, which is hereby incorporated herein by reference in its entirety). For example, the seq2hla tool (see, e.g., Boegel et al., “HLA typing from RNA-Seq sequence reads,” Genome Med. 2012; 4:102, which is hereby incorporated herein by reference in its entirety), which is well designed to perform the method as herein disclosed is an in silico method written in python and R, which takes standard RNA-seq sequence reads in fastq format as input, uses a bowtie index (Langmead B, et al., “Ultrafast and memory-efficient alignment of short DNA sequences to the human genome,” Genome Biol. 2009, 10: R25-10.1186/gb-2009-10-3-r25, which is hereby incorporated herein by reference in its entirety) comprising all HLA alleles and outputs the most likely HLA class I and class II genotypes (in 4 digit resolution), a p-value for each call, and the expression of each class. In some embodiments, HLA typing is performed using the tool Short Oligonucleotide Analysis Package-HLA (SOAP).

Referring to block 342, in some embodiments, the method further includes determining the class I HLA type of the human subject using a polymerase chain reaction using a biological sample from the cancer subject.

For instance, in some embodiments, HLA typing is performed using the sequence reads by either low to intermediate resolution polymerase chain reaction-sequence-specific primer (PCR-SSP) method or by high-resolution SeCore HLA sequence-based typing method (HLA-SBT) (INVITROGEN). In some embodiments ATHLATES is used for HLA typing and confirmation. See, e.g., Liu and Duffy et al., 2013, “ATHLATES: accurate typing of human leukocyte antigen through exome sequencing,” Nucleic Acids Res 41: e142, which is hereby incorporated herein by reference in its entirety.

### Selecting Neoantigens for Tumor Vaccine.

Referring to block 344, the method further includes selecting, for the tumor vaccine, two or more candidate neoantigens 234 in the plurality of candidate neoantigens as a final set of neoantigens based on the respective score 242 of each candidate neoantigen 234 in the plurality of candidate neoantigens.

In some embodiments, the selecting includes selecting, as the final set of neoantigens, a subset of candidate neoantigens having the top N scores in the plurality of candidate neoantigens. In some embodiments, N is at least 2, at least 5, at least 10, at least 20, at least 50, at least 100, at least 200, at least 500, or at least 1000. In some embodiments, N is no more than 5000, no more than 1000, no more than 500, no more than 200, no more than 100, no more than 50, no more than 20, no more than 10, or no more than 5. In some embodiments, N is from 2 to 10, from 2 to 50, from 5 to 80, from 50 to 200, from 100 to 500, from 300 to 2000, or from 1000 to 5000. In some embodiments, N falls within another range starting no lower than 2 and ending no higher than 5000.

In some embodiments, the selecting includes selecting, as the final set of neoantigens, a subset of candidate neoantigens having the top N percentage of scores in the plurality of candidate neoantigens. In some embodiments, N is at least 5, at least 10, at least 15, at least 20, at least 30, at least 40, at least 50, or at least 80 percent. In some embodiments, N is no more than 100, no more than 80, no more than 50, no more than 30, no more than 20, no more than 10, or no more than 5 percent. In some embodiments, N is from 5 to 25, from 20 to 60, from 40 to 80, from 50 to 90, or from 70 to 100 percent. In some embodiments, N falls within another range starting no lower than 5 percent and ending no higher than 100 percent.

In some embodiments, the method further includes ranking the plurality of candidate neoantigens based on the respective score of each candidate neoantigen in the plurality of candidate neoantigens.

Referring to block 346, in some embodiments, the final set of neoantigens consists of between two and twenty neoantigens in the plurality of candidate neoantigens.

In some embodiments, the final set of neoantigens includes at least 2, at least 5, at least 10, at least 20, at least 50, at least 100, at least 200, at least 500, or at least 1000 neoantigens. In some embodiments, the final set of neoantigens includes no more than 5000, no more than 1000, no more than 500, no more than 200, no more than 100, no more than 50, no more than 20, no more than 10, or no more than 5 neoantigens. In some embodiments, the final set of neoantigens consists of from 2 to 10, from 2 to 50, from 5 to 80, from 50 to 200, from 100 to 500, from 300 to 2000, or from 1000 to 5000 neoantigens. In some embodiments, the final set of neoantigens falls within another range starting no lower than 2 neoantigens and ending no higher than 5000 neoantigens.

Referring to block 348, in some embodiments, the final set of neoantigens consists of one or more neoantigens from the first subset of the plurality of candidate neoantigens and one or more neoantigens from the second subset of the plurality of candidate neoantigens.

In some embodiments, the final set of neoantigens consists of at least 2, at least 5, at least 10, at least 20, at least 50, at least 100, at least 200, at least 500, or at least 800 neoantigens from the first subset of the plurality of candidate neoantigens. In some embodiments, the final set of neoantigens consists of no more than 1000, no more than 800, no more than 500, no more than 200, no more than 100, no more than 50, no more than 20, no more than 10, or no more than 5 neoantigens from the first subset of the plurality of candidate neoantigens. In some embodiments, the final set of neoantigens consists of from 2 to 10, from 2 to 50, from 5 to 80, from 50 to 200, from 100 to 500, or from 300 to 1000 neoantigens from the first subset of the plurality of candidate neoantigens. In some embodiments, the final set of neoantigens falls within another range starting no lower than 2 neoantigens and ending no higher than 1000 neoantigens from the first subset of the plurality of candidate neoantigens.

Alternatively or additionally, in some embodiments, the final set of neoantigens consists of at least 2, at least 5, at least 10, at least 20, at least 50, at least 100, at least 200, at least 500, or at least 800 neoantigens from the second subset of the plurality of candidate neoantigens. In some embodiments, the final set of neoantigens consists of no more than 1000, no more than 800, no more than 500, no more than 200, no more than 100, no more than 50, no more than 20, no more than 10, or no more than 5 neoantigens from the second subset of the plurality of candidate neoantigens. In some embodiments, the final set of neoantigens consists of from 2 to 10, from 2 to 50, from 5 to 80, from 50 to 200, from 100 to 500, or from 300 to 1000 neoantigens from the second subset of the plurality of candidate neoantigens. In some embodiments, the final set of neoantigens falls within another range starting no lower than 2 neoantigens and ending no higher than 1000 neoantigens from the second subset of the plurality of candidate neoantigens.

Alternatively or additionally, in some embodiments, the final set of neoantigens consists of at least 2, at least 5, at least 10, at least 20, at least 50, at least 100, at least 200, at least 500, or at least 800 neoantigens from the third subset of the plurality of candidate neoantigens. In some embodiments, the final set of neoantigens consists of no more than 1000, no more than 800, no more than 500, no more than 200, no more than 100, no more than 50, no more than 20, no more than 10, or no more than 5 neoantigens from the third subset of the plurality of candidate neoantigens. In some embodiments, the final set of neoantigens consists of from 2 to 10, from 2 to 50, from 5 to 80, from 50 to 200, from 100 to 500, or from 300 to 1000 neoantigens from the third subset of the plurality of candidate neoantigens. In some embodiments, the final set of neoantigens falls within another range starting no lower than 2 neoantigens and ending no higher than 1000 neoantigens from the third subset of the plurality of candidate neoantigens.

Referring to block 350, in some embodiments, the final set of neoantigens consists of one or more neoantigens from the first subset of the plurality of candidate neoantigens, one or more neoantigens from the second subset of the plurality of candidate neoantigens, and one or more neoantigens from the third subset of the plurality of candidate neoantigens.

### Additional Scoring Terms.

As described above, in some embodiments, the method includes determining a respective score for each respective candidate neoantigen in the plurality of candidate neoantigens using a scoring function that includes a first scoring term that upweights respective candidate neoantigens having a higher class I major histocompatibility complex (MHC) affinity relative to respective candidate neoantigens having a lower class I MHC affinity, given a class I human leukocyte antigen (HLA) type of the human subject.

Alternatively or additionally, in some embodiments, the method further includes obtaining one or more additional scoring terms for the scoring function that is used to determine the respective score for each respective candidate neoantigen in the plurality of candidate neoantigens. As an illustrative example, in some implementations, the scoring function utilizes a “neoantigen quality” score that combines biophysical, chemical, and computationally inferred properties of a candidate neoantigen that make it more likely to induce a productive immune response against the tumor. In some implementations, these properties include, but are not limited to, affinity of a neoantigen to MHC, avidity of the peptide-MHC complex to the recognizing TCR, type of T cells responding to the neoantigen and sequence similarity to known highly immunogenic epitopes. Advantageously, and without being limited to any one theory of operation, sequence similarity is thought to play a role in segregating responders to checkpoint therapy but is not usually considered in algorithms of neoantigen prediction.

Allele-specific expression. Referring to block 352, in some embodiments, the method further includes determining a respective allele-specific expression of each somatic variant in the first plurality of variants using the first plurality of sequence reads. In some such embodiments, the scoring function further includes a second scoring term that upweights respective candidate neoantigens representing alleles with higher abundance in the first plurality of sequence reads relative to respective candidate neoantigens representing alleles with lower abundance in the first plurality of sequence reads.

Generally, allele-specific expression (ASE) refers to a phenomenon that occurs in diploid or polypoid genomes, in which two or more alleles of a gene exhibit imbalanced expression. In some cases, certain alleles of a given gene or locus are known to be preferentially associated with disease (e.g., disease-associated alleles). Allele-specific expression has been observed in tumors. In addition to an association with cancer risk, tumor initiation and progression, ASE also affects the prognosis and outcome of cancer patients. See, for example, Liu Z, Dong X, Li Y. A genome-wide study of allele-specific expression in colorectal cancer. Front Genet. 2018; 9, which is hereby incorporated herein by reference in its entirety.

Accordingly, in some embodiments, the method includes using RNA sequence reads to determine the relative abundance of particular alleles of respective loci (e.g., carrying respective somatic variants) and upweight neoantigens that are more abundant in the subject based on such allele abundances.

In some embodiments, the second scoring term upweights respective candidate neoantigens representing alleles with lower abundance in the first plurality of sequence reads relative to respective candidate neoantigens representing alleles with higher abundance in the first plurality of sequence reads.

Alternatively or additionally, in some embodiments, the method further includes determining a respective allele-specific expression of each fusion protein in the one or more fusion proteins using the first plurality of sequence reads. In some such embodiments, the scoring function further includes a second scoring term that upweights respective candidate neoantigens representing alleles with higher abundance in the first plurality of sequence reads relative to respective candidate neoantigens representing alleles with lower abundance in the first plurality of sequence reads. In some embodiments, the second scoring term upweights respective candidate neoantigens representing alleles with lower abundance in the first plurality of sequence reads relative to respective candidate neoantigens representing alleles with higher abundance in the first plurality of sequence reads.

In some embodiments, the scoring function does not include a scoring term for allele-specific expression.

N-terminus mutations. In some embodiments, the method further includes removing, from the plurality of candidate neoantigens, or from the final set of candidate neoantigens, those candidate neoantigens that are likely to cross-react with their wildtype counterpart (e.g., wildtype antigen). Without being limited to any one theory of operation, immunogenicity testing of subjects suggested that MHC class I epitopes harboring a mutation at their N-terminus (the first position in the MHC class I-bound peptide) are more likely to cross-react with wildtype antigen than are epitopes with mutations elsewhere in the MHC-bound peptide. This observation is consistent with MHC structural data indicating that the first position of the bound peptide is generally buried and unavailable to make TCR interactions. Accordingly, in some implementations, the systems and methods disclosed herein include removing such candidate neoantigens from the plurality of candidate neoantigens, or from the final set of candidate neoantigens for use in a tumor vaccine. Advantageously, this removal potentially avoids dangerous or ineffective responses that cross-react with wildtype sequence.

Referring to block 354, in some embodiments, the method further includes excluding from the plurality of candidate neoantigens, or from the final set of candidate neoantigens, those candidate neoantigens that include a mutation from wildtype sequence at the N-terminal residue position (e.g., position 1).

In some embodiments, the method includes retaining, in the plurality of candidate neoantigens, or in the final set of candidate neoantigens, those candidate neoantigens that include a mutation from wildtype sequence at position 2, 3, 4, 5, 6, 7, 8, 9, 10, 11, 12, 13, or 14. In some embodiments, the method includes retaining, in the plurality of candidate neoantigens, or in the final set of candidate neoantigens, those candidate neoantigens that include a mutation from wildtype sequence at position 2 or position 9. In some embodiments, the method includes retaining, in the plurality of candidate neoantigens, or in the final set of candidate neoantigens, those candidate neoantigens that include a mutation from wildtype sequence at a T cell receptor (TCR)-contacting position. Alternatively or additionally, in some embodiments, the cancer is glioblastoma.

In some embodiments, the method does not include excluding candidate neoantigens that include a mutation from wildtype sequence at the N-terminal residue position (e.g., position 1).

Hydrophobicity. Referring to block 356, in some embodiments, the method further includes determining a hydrophobicity of each respective candidate neoantigen in the plurality of candidate neoantigens, and the scoring function further includes a scoring term for hydrophobicity of the respective candidate neoantigen. Without being limited to any one theory of operation, immunogenicity testing of subjects afflicted with cancer indicated that hydrophobic peptides are more likely to elicit a T cell response. This is thought to be due to enhanced aggregation potential or improved peptide/TCR interactions. Accordingly, in some embodiments, the selection criteria for candidate neoantigens for tumor vaccines prioritize hydrophobic peptides (e.g., candidate neoantigens).

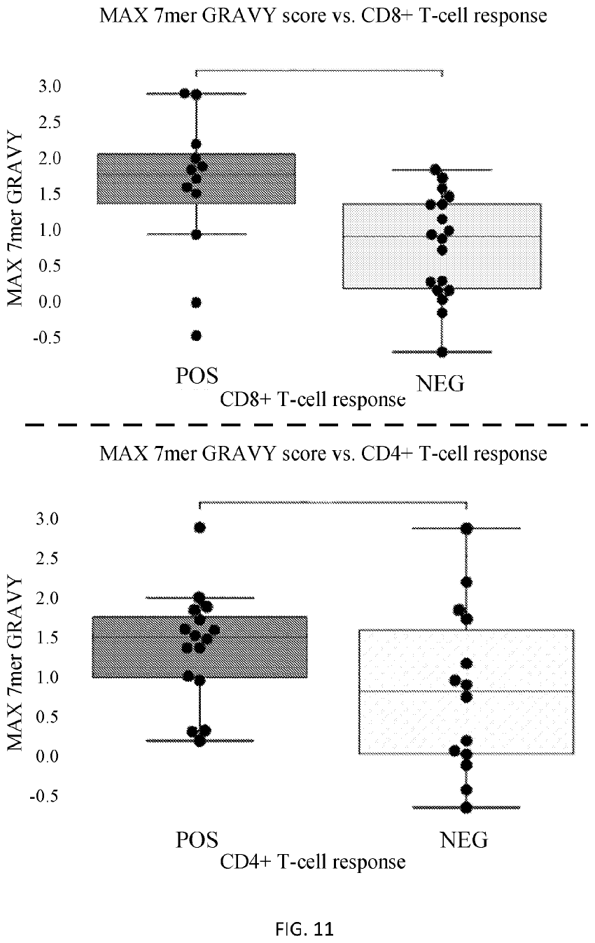

In an example embodiment, a plurality of candidate neoantigens is considered. A respective score for each respective candidate neoantigen in the plurality of candidate neoantigens can be determined using a scoring function including at least a scoring term that upweights candidate neoantigens having higher hydrophobicity relative to candidate neoantigens having lower hydrophobicity. In particular, as illustrated in FIG. 11, peptide hydrophobicity of candidate neoantigens can be determined as a predictor of T cell responses. Subjects (data points) enrolled in a clinical trial can be assessed for peptide hydrophobicity of candidate neoantigens determined for each of the respective subjects in order to predict CD8+ T cell responses. In general, candidate neoantigens that are more hydrophobic can be deemed to be more likely to stimulate a CD8+ response. Peptide hydrophobicity can be quantified by computing the maximum value of the GRAVY score across 7-mers within the long peptide (e.g., the candidate neoantigen), for each of 30 candidate neoantigens. Modeling plots show that the maximum GRAVY scores for 7-mers could be predictive for CD8+ (top panel) but not CD4+ (bottom panel) responses. For CD8+ responses, the scoring term for upweighting candidate neoantigens having higher hydrophobicity relative to candidate neoantigens having lower hydrophobicity are assigned as a threshold maximum GRAVY score of 0.5 (top panel, dashed line), such that candidate neoantigens having a maximum GRAVY score of greater than 0.5 are prioritized relative to candidate neoantigens having a maximum GRAVY score of less than or equal to 0.5. Methods and tools for determining the hydrophobicity of proteins and peptides (e.g., antigens) are known in the art. For example, the grand average of hydropathicity index (GRAVY) is a public domain program for calculating hydrophobicity. GRAVY is used to represent the hydrophobicity value of a peptide, which calculates the sum of the hydropathy values of all the amino acids divided by the sequence length. In some embodiments, the hydrophobicity of a respective candidate neoantigen is determined using a software tool (e.g., ExPASy, ProPAS, etc.). In some embodiments, the hydrophobicity of a respective candidate neoantigen is determined using a kit (e.g., a protein analysis kit and/or a peptide assay kit). In some embodiments, the determining the respective hydrophobicity of each candidate neoantigen in the plurality of candidate neoantigens comprises determining the maximum hydrophobicity score across a set of residues in the respective candidate neoantigen. In some such embodiments, the determining of the respective hydrophobicity of each candidate neoantigen in the plurality of candidate neoantigens further includes filtering the plurality of candidate neoantigens by a threshold maximum hydrophobicity score.

In some embodiments, the set of residues comprises at least 2, at least 3, at least 4, at least 5, at least 6, at least 7, at least 8, at least 9, at least 10, at least 11, at least 12, or at least 13 residues. In some embodiments, the set of residues comprises no more than 15, no more than 13, no more than 10, no more than 8, no more than 5, or no more than 3 residues. In some embodiments, the set of residues consists of from 2 to 8, from 4 to 10, from 6 to 12, or from 8 to 15 residues. In some embodiments, the set of residues falls within another range starting no lower than 2 residues and ending no higher than 15 residues.

In some embodiments, the set of residues comprises at least 10, at least 20, at least 50, at least 60, or at least 80 percent of the length in residues of the respective candidate neoantigen. In some embodiments, the set of residues comprises no more than 100, no more than 80, no more than 60, or no more than 50 percent of the length in residues of the respective candidate neoantigen. In some embodiments, the set of residues consists of from 10 to 30, from 20 to 50, from 40 to 60, from 50 to 80, or from 70 to 100 percent of the length in residues of the respective candidate neoantigen. In some embodiments, the set of residues falls within another range of the length in residues of the respective candidate neoantigen starting no lower than 10 percent and ending no higher than 100 percent.

Referring to block 358, in some embodiments, the determining the respective hydrophobicity of each candidate neoantigen in the plurality of candidate neoantigens comprises assigning the respective candidate neoantigen the maximum hydrophobicity score of any 7-mer within the respective candidate neoantigen.

In some embodiments, the threshold maximum hydrophobicity score is at least 0.5. In some embodiments, the threshold maximum hydrophobicity score is at least 0.2, at least 0.3, at least 0.5, or at least 0.8. In some embodiments, the threshold maximum hydrophobicity score is no more than 1, no more than 0.8, no more than 0.5, or no more than 0.3. In some embodiments, the threshold maximum hydrophobicity score is from 0.2 to 0.6, from 0.4 to 0.8, or from 0.6 to 1. In some embodiments, the threshold maximum hydrophobicity score falls within another range starting no lower than 0.2 and ending no higher than 1.

Referring to block 360, in some embodiments, the scoring term for hydrophobicity of the respective candidate neoantigen upweights more hydrophobic candidate neoantigens relative to less hydrophobic candidate neoantigens.

In some embodiments, the scoring function does not include a scoring term for hydrophobicity of the respective candidate neoantigen.

Class II MHC affinity. In some embodiments, the method further includes generating improved CD4 epitope prediction through incorporation of class II MHC binding prediction. Generally, existing approaches have focused on class I MHC binding prediction to elicit a CD8+ T cell response. However, and without being limited to any one theory of operation, accumulating evidence suggests that a vaccine induced CD4+ T cell response can be an important determinant of antitumor immunity. Thus, in some implementations, the method further includes applying additional ranking criteria that incorporate class II MHC binding prediction using class II MHC affinity prediction tools (e.g., NetMHCIIpan).

Referring to block 362, in some embodiments, the scoring function further includes a term for class II MHC affinity of the respective candidate neoantigen, given a class II HLA type of the human subject, that upweights respective candidate neoantigens having higher class II MHC affinity than respective candidate neoantigens having lower class II MHC affinity.

Typically, peptides presented by class II MHC molecules are derived from extracellular proteins (not cytosolic as in class I MHC), mainly of bacterial origin. They are endocytosed by professional antigen presenting cells (APCs) such as dendritic cells, macrophages, and B-cells, digested in lysosomes by cathepsin S, and bound by class IImolecules in subcellular vesicles. The complex peptide-class II molecule is then expressed on the cell surface to interact exclusively with CD4+T cells (helper T cells, THC). TH cells help to trigger an appropriate immune response which may include localized inflammation and swelling due to recruitment of phagocytes or may lead to a full-force antibody-mediated immune response due to the activation of B cells.

The successful prediction of class II MHC binding is often more difficult than the successful prediction of class I binding. One major difficulty is the unrestricted length of class II epitopes leading to promiscuous class II MHC binding. Compared with class I MHC binders, which are limited up to 11 amino acids, though sometimes longer, the open-ended class II binding site does not constrain peptide lengths, allowing binding of peptides consisting of up to or more than 25 amino acids.

Methods for predicting peptide-MHC binding are known in the art. In particular, experimentally determined affinities data have formed the basis of many peptide-MHC binding prediction methods, which are able effectively to discriminate binding from nonbinding peptides. Such methods include motifs, algorithms (e.g., artificial neural networks, hidden Markov models (HMMs), and/or support vector machines (SVMs)), and/or computational chemistry methods (e.g., QSAR analysis and/or structure-based approaches). Suitable methods for predicting class II MHC binding affinity further include approaches for resolving the dynamic variable-length problem inherent within the class II prediction, such as iterative “meta-search” algorithm, Ant Colony search, Gibbs sampling algorithm, and/or multi-objective evolutionary algorithm.

Example non-limiting tools for predicting class II MHC binding affinity that are contemplated for use in the present disclosure include NetMHCIIpan, NetMHCII, ProPred, RANKPEP, EpiTOP, IEDB-ARB, IEDB-SMM, and/or MHC2Pred. ProPred predicts MHC class II binding peptides using quantitative matrix-based pocket profiles. RANKPEP uses position-specific scoring matrices (PSSM) or profiles which represent the observed sequence-weighted frequency of all amino acids in every position of a sequence alignment. IEDB-ARB is a matrix-based prediction method where the peptide binding score is calculated by multiplying the relative contribution coefficients for each amino acid at each peptide position. The IEDB-SMM align method is based on an integrated alignment and motif identification algorithm and predicts direct peptide binding affinities. MHC2Pred is an SVM-based prediction server. EpiTOP is a newly developed method for MHC class II binding prediction based on proteochemometrics. It is a matrix-based method which considers both peptide and protein binding site amino acids contributions. NetMHCII and NetMHCIIpan are ANN-based methods. NetMHCIIpan considers both peptide and MHC sequence information.

Methods for predicting peptide-MHC binding are further described in Dimitrov et al., “MHC class II binding prediction-a little help from a friend,” J Biomed Biotechnol. 2010; 2010:705821, which is hereby incorporated herein by reference in its entirety.

### Vaccine Preparation and Treatment.

Referring to block 364, in some embodiments, the method further includes forming tumor vaccine by solubilizing the final set of neoantigens in a carrier, and administering the vaccine to the human subject.

In some embodiments, the forming the tumor vaccine further includes adding one or more adjuvants to the tumor vaccine. Typically, vaccine adjuvants are compounds used to increase the immunogenicity of a given antigen. They serve to enhance the magnitude, breadth, quality, and longevity of specific immune responses to antigens but have minimal toxicity or lasting immune effects on their own. In some implementations, effective adjuvants function to activate the innate immune system, such as through TLR signaling. In some embodiments, the one or more adjuvants are selected from the group consisting of Polyinosinic-Polycytidylic Acid stabilized with Polylysine and Carboxymethylcellulose (Poly-ICLC), montanide, and/or Keyhole Limpet Hemocyanin (KLH).

Referring to block 366, in some embodiments, the forming further comprises including poly-ICLC in the tumor vaccine. In some embodiments, the tumor vaccine does not include poly-ICLC.

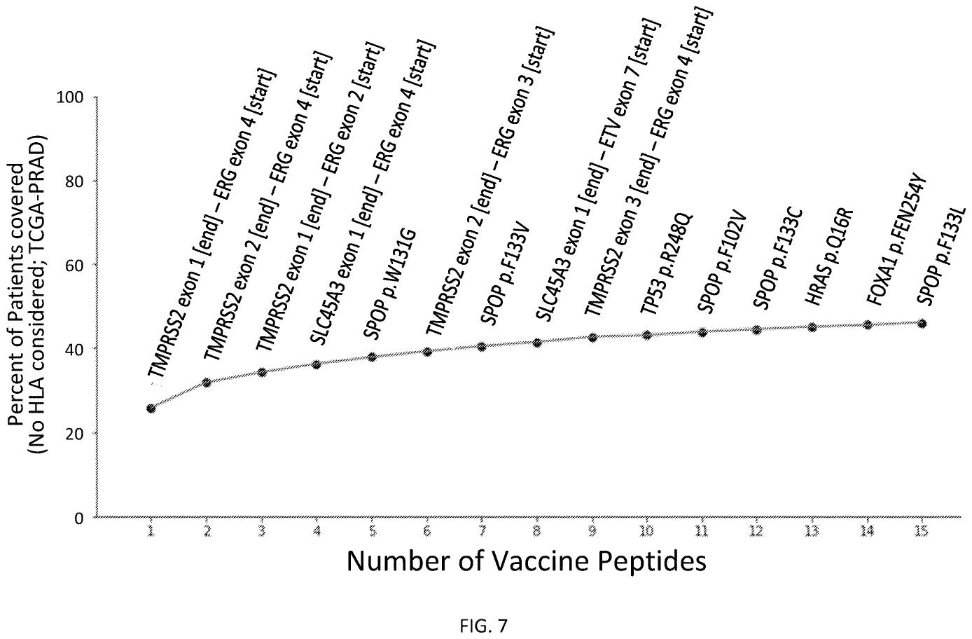

In some embodiments, the tumor vaccine includes at least a first neoantigen that encodes a somatic variant (e.g., a single nucleotide polymorphism), and a second neoantigen that encodes a fusion protein (e.g., one or more residues from a portion of a first human protein and one or more residues from a portion of a second human protein). Advantageously, as described in Example 2 with reference to FIG. 7, in some implementations, the use of tumor vaccines that include neoantigens encoding somatic variants and neoantigens encoding fusion proteins improves the coverage of human subjects vaccinated against cancer neoantigens (e.g., prostate cancer), in a population of human subjects afflicted with cancer.

In some embodiments, the cancer is prostate cancer and the second neoantigen encodes a fusion represented in Table 1. See, for example, the section entitled “5.4 Example Embodiments for Neoantigen-Based Tumor Vaccines,” below.

In some embodiments, the cancer is prostate cancer, and the single nucleotide polymorphism is in SPOP, TP53, FOXA1 or PTEN.

Any suitable method for forming and/or administering a tumor vaccine targeting the final set of neoantigens is contemplated for use in the present disclosure. See, e.g., the sections entitled “4. Therapeutic Uses of the Neoantigens,” “4.1 Vaccines,” and “4.2 Adoptive T cell Therapy,” above. For example, in some implementations, the method further includes forming tumor vaccine by encoding the final set of neoantigens in mRNA and/or DNA. In some embodiments, a respective neoantigen in the final set of neoantigens is obtained as (e.g., encoded in) one or more RNA molecules and/or one or more DNA molecules. In some embodiments, each respective neoantigen in the final set of neoantigens is obtained as (e.g., encoded in) one or more RNA molecules and/or one or more DNA molecules. Alternatively or additionally, in some embodiments, a respective neoantigen in the plurality of candidate neoantigens (e.g., in the first subset of candidate neoantigens and/or in the second subset of candidate neoantigens) is obtained as (e.g., encoded in) one or more RNA molecules and/or one or more DNA molecules. In some embodiments, each respective neoantigen in the plurality of candidate neoantigens (e.g., in the first subset of candidate neoantigens and/or in the second subset of candidate neoantigens) is obtained as (e.g., encoded in) one or more RNA molecules and/or one or more DNA molecules. In some such embodiments, the mRNA and/or DNA is encased in a plasmid, vector (e.g., a viral vector, such as an adenovirus vector), and/or nanoparticle (e.g., a lipid nanoparticle) for delivery. In some embodiments, the tumor vaccine comprises a self-amplifying mRNA construct. In some embodiments, the method further includes administering the tumor vaccine in a plasmid or vector form. In some embodiments, the tumor vaccine is administered via electroporation (e.g., into muscle). In some embodiments, the tumor vaccine is injected into the tumor directly via intra-tumor injection.

In some embodiments, the vaccine is an antigen presenting cell vaccine, e.g., a dendritic cell vaccine. See, e.g., the sections entitled “4. Therapeutic Uses of the Neoantigens,” “4.1 Vaccines,” and “4.2 Adoptive T cell Therapy,” above.

In some embodiments of the present disclosure, the neoantigens of the present disclosure are used in adoptive T cell therapy. See, e.g., the section entitled “4.2 Adoptive T cell Therapy,” above.

In some embodiments, the present disclosure provides a method of inducing or eliciting an antitumor response or improving or enhancing antitumor T cell immunity in a subject in need thereof, the method comprising administering an effective amount of a tumor vaccine, or any embodiments thereof, as disclosed herein. In some embodiments, the present disclosure provides a method of preventing, treating, reducing, or slowing progression or development of a cancer in a subject in need thereof, the method comprising administering an effective amount of a tumor vaccine, or any embodiments thereof, as disclosed herein.

Any suitable methods of administering a neoantigen as described herein, or compositions or vaccines containing the neoantigens, to a subject are contemplated for use in the present disclosure. In some embodiments, the neoantigens, compositions, and/or vaccines are administered to a subject by any suitable route, e.g., oral, nasal, buccal (e.g., sub-lingual), intratumoral, parenteral (e.g., subcutaneous, intracutaneous, intraocular, intranasal, intraperitoneal intramuscular, intradermal, or intravenous), topical (i.e., both skin and mucosal surfaces, including airway surfaces), rectal, vaginal, sublingual, intra-tracheal, transmucosal, pulmonary, and/or transdermal administration. In an embodiment, the neoantigens, compositions, and/or vaccines are administered systemically by intravenous injection or parenterally by subcutaneous (e.g., superficial subcutaneous) injection. In another embodiment, the neoantigens, compositions, and/or vaccines are administered directly to a target site, by, for example, surgical delivery to an internal or external target site, or by catheter to a site accessible by a blood vessel. If administered via intravenous injection, in some implementations, the neoantigens, compositions, and/or vaccines are administered in a single bolus, multiple injections, or by continuous infusion (e.g., intravenously, by peritoneal dialysis, pump infusion). In some embodiments, the neoantigens, compositions, and/or vaccines provided herein are administered either systemically or locally (e.g., directly). Non-limiting examples of systemic administration include oral, transdermal, subdermal, intraperitioneal, subcutaneous, transnasal, sublingual, and/or rectal administration. Alternatively, in some embodiments, the neoantigens, compositions, and/or vaccines provided herein are delivered via a sustained delivery device implanted, for example, subcutaneously or intramuscularly. In some embodiments, the neoantigens, compositions, and/or vaccines provided herein are administered by continuous release or delivery, using, for example, an infusion pump, continuous infusion, controlled release formulations utilizing polymer, oil, and/or water-insoluble matrices. In some embodiments, the neoantigens, compositions, and/or vaccines provided herein comprise a formulation that is selected for the mode of delivery, including but not limited to any of the modes of delivery disclosed herein.

In some embodiments, the method further includes administering a second anti-cancer agent to the subject, where the anti-cancer agent is administered simultaneously or sequentially. Anti-cancer agents include, e.g., anti-neoplastic agents, anti-tumor agents, anti-angiogenic agents, and immunotherapeutic agents. In some embodiments, the method further includes administering a checkpoint blockade drug to the subject, where the checkpoint blockade drug is administered simultaneously or sequentially. A list of suitable second anti-cancer agents contemplated for use in the present disclosure is included in U.S. Patent Application Publication No. US 2017/0151240, which is incorporated herein by reference in its entirety.

In some embodiments where the neoantigens, vaccines, and/or compositions described herein are administered as part of a combination therapy with any other anti-cancer agent in the methods described herein, a first composition (e.g., the tumor vaccine) is administered at the same time point or approximately the same time point as a second composition (e.g., the second anti-cancer agent). Alternatively, in some embodiments, the first and second compositions are administered at different time points. In some embodiments, the neoantigens, vaccines, and/or compositions described herein are used in a combination therapy that includes one or more of immunotherapy, chemotherapy, radiotherapy, and surgery.

Referring to block 368, in some embodiments, the administering is repeated a plurality of times over a plurality of months.

In some embodiments, the plurality of times includes at least 2, at least 5, at least 10, at least 20, at least 50, at least 100, or at least 200 times. In some embodiments, the plurality of times includes no more than 500, no more than 200, no more than 100, no more than 50, no more than 20, no more than 10, or no more than 5 times. In some embodiments, the plurality of times consists of from 2 to 10, from 2 to 50, from 5 to 80, from 50 to 200, or from 100 to 500 times. In some embodiments, the plurality of times falls within another range starting no lower than 2 times and ending no higher than 500 times.

In some embodiments, the plurality of months includes at least 2, at least 3, at least 6, at least 9, at least 12, at least 18, at least 24, at least 30, at least 36, at least 48, at least 60, at least 72, at least 84, at least 96, at least 108, or at least 120 months. In some embodiments, the plurality of months includes no more than 180, no more than 120, no more than 60, no more than 48, no more than 36, no more than 24, no more than 12, or no more than 6 months. In some embodiments, the plurality of months consists of from 3 to 12, from 12 to 24, from 24 to 36, from 36 to 60, from 60 to 120, or from 120 to 180 months. In some embodiments, the plurality of months falls within another range starting no lower than 2 and ending no higher than 180.

In some embodiments, the neoantigens as described herein, or compositions or vaccines containing the neoantigens, are administered to a subject in need thereof (e.g., a human subject afflicted by a cancer) in an effective amount, that is, an amount capable of producing a target result in a treated individual. Target results include one or more of, for example, inducing or enhancing an immune response, reducing tumor size, reducing cancer cell metastasis, and/or prolonging survival. Such a therapeutically effective amount can be determined according to standard methods. Toxicity and therapeutic efficacy of the neoantigens as described herein, or compositions or vaccines containing the neoantigens, that are utilized in the methods described herein can be determined by standard pharmaceutical procedures. As is well known in the medical and veterinary arts, dosage for any one individual depends on many factors, including the individual's size, body surface area, age, the particular composition to be administered, time and route of administration, general health, and other drugs being administered concurrently. A delivery dose of a composition as described herein is determined based on preclinical efficacy and safety.

In another embodiment, described herein are kits for inducing or enhancing an immune response and for treating cancer in a subject. A typical kit includes a composition including a pharmaceutically acceptable carrier (e.g., a physiological buffer) and a therapeutically effective amount of at least one neoantigen as described herein, or composition or vaccine containing the neoantigen; and instructions for use. A kit can also include a second anti-cancer agent. Kits also typically include a container and packaging. Instructional materials for preparation and use of the peptides, vaccines and compositions described herein are generally included. While the instructional materials typically include written or printed materials, they are not limited to such. Any medium capable of storing such instructions and communicating them to an end user is encompassed by the kits herein. Such media include, but are not limited to electronic storage media, optical media, and the like. Such media may include addresses to internet sites that provide such instructional materials.

### 5.3 Additional Embodiments

Another aspect of the present disclosure includes a system for identifying a tumor vaccine personalized to a human subject afflicted with a cancer, including a memory; one or more processors; and one or more modules stored in the memory and configured for execution by the one or more processors. The one or more modules include instructions for (A) determining a first plurality of somatic variants of the subject; (B) obtaining, in electronic form, a first plurality of sequence reads from RNA molecules in a sample of a tumor obtained from the subject; and (C) determining, from the first plurality of sequence reads, one or more fusion proteins encoded by the first plurality of sequence reads, wherein each respective fusion protein in the one or more fusion proteins is a fusion of a portion of a respective first human protein and a portion of a respective second human protein. The one or more modules further include instructions for (D) selecting a plurality of candidate neoantigens comprising a first subset of candidate neoantigens and a second subset of candidate neoantigens, where each neoantigen in the first subset of candidate neoantigens encodes a somatic variant in the first plurality of somatic variants, and each neoantigen in the second subset of candidate neoantigens encodes one or more residues from the portion of the respective first human protein and one or more residues from the portion of the respective second human protein. The one or more modules further include instructions for (E) determining a respective score for each respective candidate neoantigen in the plurality of candidate neoantigens using a scoring function that includes a first scoring term that upweights respective candidate neoantigens having a higher class I major histocompatibility complex (MHC) affinity relative to respective candidate neoantigens having a lower class I MHC affinity, given a class I human leukocyte antigen (HLA) type of the human subject; and (F) selecting, for the tumor vaccine, two or more candidate neoantigens in the plurality of candidate neoantigens as a final set of neoantigens based on the respective score of each candidate neoantigen in the plurality of candidate neoantigens.

Another aspect of the present disclosure includes a non-transitory computer readable storage medium for identifying a tumor vaccine personalized to a human subject afflicted with a cancer, the non-transitory computer readable storage medium storing one or more programs for execution by one or more processors of a computer system. The one or more computer programs includes instructions for (A) determining a first plurality of somatic variants of the subject; (B) obtaining, in electronic form, a first plurality of sequence reads from RNA molecules in a sample of a tumor obtained from the subject; and (C) determining, from the first plurality of sequence reads, one or more fusion proteins encoded by the first plurality of sequence reads, where each respective fusion protein in the one or more fusion proteins is a fusion of a portion of a respective first human protein and a portion of a respective second human protein. The one or more modules further include instructions for (D) selecting a plurality of candidate neoantigens comprising a first subset of candidate neoantigens and a second subset of candidate neoantigens, where each neoantigen in the first subset of candidate neoantigens encodes a somatic variant in the first plurality of somatic variants, and each neoantigen in the second subset of candidate neoantigens encodes one or more residues from the portion of the respective first human protein and one or more residues from the portion of the respective second human protein. The one or more modules further include instructions for (E) determining a respective score for each respective candidate neoantigen in the plurality of candidate neoantigens using a scoring function that includes a first scoring term that upweights respective candidate neoantigens having a higher class I major histocompatibility complex (MHC) affinity relative to respective candidate neoantigens having a lower class I MHC affinity, given a class I human leukocyte antigen (HLA) type of the human subject; and (F) selecting, for the tumor vaccine, two or more candidate neoantigens in the plurality of candidate neoantigens as a final set of neoantigens based on the respective score of each candidate neoantigen in the plurality of candidate neoantigens.

Yet another aspect of the present disclosure includes a system including a memory; one or more processors; and one or more modules stored in the memory and configured for execution by the one or more processors, the one or more modules including instructions for performing any of the methods disclosed herein.

Still another aspect of the present disclosure includes a non-transitory computer readable storage medium, the non-transitory computer readable storage medium storing one or more programs for execution by one or more processors of a computer system, the one or more computer programs including instructions for performing any of the methods disclosed herein.

### 5.4 Example Embodiments for Neoantigen-Based Tumor Vaccines

Yet another aspect of the present disclosure provides a tumor vaccine including a plurality of neoantigenic peptides and an adjuvant personalized for a subject afflicted with a cancer, where a first neoantigen in the tumor vaccine encodes a single nucleotide polymorphism present in RNA molecules in a tumor biopsy obtained from the subject, and a second neoantigen in the tumor vaccine encodes one or more residues from a portion of a first human protein and one or more residues from a portion of a second human protein, where the tumor biopsy includes RNA molecules that are a fusion of the first human protein and the second human protein.

In some embodiments, the cancer is prostate cancer and the second neoantigen encodes a fusion represented in Table 1.

TABLE 1
Prostate Cancer Fusions

| Fusion | | |
|---|---|---|
| Protein | Identify of | Identity of |
| Number | First Protein | Second Protein |
| 1 | KANSL1 | ARL17A |
| 2 | EIF3B | FOXK2 |
| 3 | TMPRSS2 | ERG |
| 4 | TMPRSS2 | ETV1 |
| 5 | TMPRSS2 | ETV4 |
| 6 | SLC45A3 | ERG |
| 7 | SLC45A3 | ETV |
| 8 | SLC45A3 | BRAF |
| 9 | ESRP1 | RAF1 |

In some embodiments, the cancer is prostate cancer, and the single nucleotide polymorphism is in SPOP, TP53, FOXA1 or PTEN.

In some embodiments, the cancer is a carcinoma, a melanoma, a lymphoma/leukemia, a sarcoma, or a neuro-glial tumor. In some embodiments, the cancer is lung cancer, pancreatic cancer, colon cancer, stomach or esophagus cancer, breast cancer, ovary cancer, prostate cancer, or liver cancer.

In some embodiments, the tumor biopsy is a fresh frozen sample.

In some embodiments, each respective neoantigenic peptide in the plurality of neoantigenic peptides has a length of N residues, wherein N is 8, 9, 10, 11, 12, 13, or 14. In some embodiments, each respective neoantigenic peptide in the plurality of neoantigenic peptides has a length of 9 residues.

In some embodiments, each neoantigenic peptide in the plurality of neoantigenic peptides does not include a mutation from wildtype sequence at the N-terminal residue position.

In some embodiments, the plurality of neoantigenic peptides is solubilized in a carrier. In some embodiments, the adjuvant is poly-ICLC.

In some embodiments, the tumor vaccine is administered to the subject. In some embodiments, the administering is repeated a plurality of times over a plurality of months.

### 5.5 Example Embodiments for Hydrophobicity-Based Neoantigen Selection

Still another aspect of the present disclosure includes a method for identifying a tumor vaccine personalized to a human subject afflicted with a cancer, the method including (A) determining a first plurality of somatic variants of the subject and (B) selecting a plurality of candidate neoantigens where each neoantigen in at least a subset of candidate neoantigens in the plurality of candidate neoantigens encodes a somatic variant in the first plurality of somatic variants. The method further includes (C) determining a respective score for each respective candidate neoantigen in the plurality of candidate neoantigens using a scoring function that comprises a first scoring term that upweights candidate neoantigens having a higher class I major histocompatibility complex (MHC) affinity relative to candidate neoantigens having a lower class I MHC affinity, given a class I human leukocyte antigen (HLA) type of the human subject, and a second scoring term that upweights candidate neoantigens having higher hydrophobicity relative to candidate neoantigens having lower hydrophobicity. The method further includes (D) selecting, for the tumor vaccine, two or more candidate neoantigens in the plurality of candidate neoantigens as a final set of neoantigens based on the respective score of each candidate neoantigen in the plurality of candidate neoantigens.

In some embodiments, the method further includes, after the determining (A), obtaining a first plurality of sequence reads from RNA molecules in a sample of a tumor obtained from the subject; and determining a respective allele-specific expression of each somatic variant in the first plurality of variants using the first plurality of sequence reads, where the scoring function further includes a third scoring term that upweights respective candidate neoantigens representing alleles with higher abundance in the first plurality of sequence reads relative to respective candidate neoantigens representing alleles with lower abundance in the first plurality of sequence reads.

In some embodiments, the method further includes determining, from the first plurality of sequence reads, one or more fusion proteins encoded by the first plurality of sequence reads, wherein each respective fusion protein in the one or more fusion proteins is a fusion of a portion of a respective first human protein and a portion of a respective second human protein, where the plurality of candidate neoantigens further comprises a second subset of candidate neoantigens, where each neoantigen in the second subset of candidate neoantigens encodes one or more residues from the portion of the respective first human protein and one or more residues from the portion of the respective second human protein.

In some embodiments, the cancer is glioblastoma or prostate cancer. In some embodiments, the cancer is a carcinoma, a melanoma, a lymphoma/leukemia, a sarcoma, or a neuro-glial tumor. In some embodiments, the cancer is lung cancer, pancreatic cancer, colon cancer, stomach or esophagus cancer, breast cancer, ovary cancer, prostate cancer, or liver cancer.

In some embodiments, each somatic variant in the first plurality of somatic variants is a single nucleotide variant or indel mutation.

In some embodiments, the tumor biopsy is a fresh frozen sample.

In some embodiments, each candidate neoantigen in the plurality of candidate neoantigens has a length of N residues, wherein N is 8, 9, 10, 11, 12, 13, or 14. In some embodiments, each candidate neoantigen in the plurality of candidate neoantigens has a length of 9 residues.

In some embodiments, the method further includes excluding from the plurality of candidate neoantigens, or from the final set of candidate neoantigens, those candidate neoantigens that include a mutation from wildtype sequence at the N-terminal residue position.

In some embodiments, the method further includes forming tumor vaccine by solubilizing the final set of neoantigens in a carrier; and administering the vaccine to the human subject. In some embodiments, the forming further comprises including poly-ICLC in the tumor vaccine. In some embodiments, the administering is repeated a plurality of times over a plurality of months.

### 5.6 Example Embodiments for Tumor-Normal Matched Variant Identification

Yet another aspect of the present disclosure includes a method for identifying a tumor vaccine personalized to a human subject afflicted with a cancer. The method includes (A) obtaining a first plurality of sequence reads from RNA molecules in a sample of a tumor obtained from the subject; (B) obtaining a second plurality of sequence reads from DNA molecules in the tumor sample obtained from the subject; (C) obtaining a third plurality of sequence reads from DNA molecules in a normal tissue sample proximate to an original location of the tumor in the subject; and (D) using the second plurality of sequence reads and the third plurality of sequence reads to identify a plurality of somatic variants of the subject by including in the plurality of somatic variants those somatic variants observed in the second plurality of sequence reads that are not observed in the third plurality of sequence reads. The method further includes (E) selecting a plurality of candidate neoantigens, where each neoantigen in at least a subset of candidate neoantigens in the plurality of candidate neoantigens encodes a somatic variant in the plurality of somatic variants; and (F) determining a respective score for each respective candidate neoantigen in the plurality of candidate neoantigens using a scoring function that comprises a first scoring term that upweights candidate neoantigens having a higher class I major histocompatibility complex (MHC) affinity relative to candidate neoantigens having a lower class I MHC affinity, given a class I human leukocyte antigen (HLA) type of the human subject. The method further includes (G) selecting, for the tumor vaccine, two or more candidate neoantigens in the plurality of candidate neoantigens as a final set of neoantigens based on the respective score of each candidate neoantigen in the plurality of candidate neoantigens.

In some embodiments, the method further includes determining, from the first plurality of sequence reads, one or more fusion proteins encoded by the first plurality of sequence reads, where each respective fusion protein in the one or more fusion proteins is a fusion of a portion of a respective first human protein and a portion of a respective second human protein. In some such embodiments, the plurality of candidate neoantigens further comprises a second subset of candidate neoantigens, where each neoantigen in the second subset of candidate neoantigens encodes one or more residues from the portion of the respective first human protein and one or more residues from the portion of the respective second human protein.

In some embodiments, the method further includes determining a respective allele-specific expression of each somatic variant in the first plurality of variants using the first plurality of sequence reads, where the scoring function further includes a second scoring term that upweights respective candidate neoantigens representing alleles with higher abundance in the first plurality of sequence reads relative to respective candidate neoantigens representing alleles with lower abundance in the first plurality of sequence reads.

In some embodiments, the cancer is glioblastoma or prostate cancer. In some embodiments, the cancer is a carcinoma, a melanoma, a lymphoma/leukemia, a sarcoma, or a neuro-glial tumor. In some embodiments, the cancer is lung cancer, pancreatic cancer, colon cancer, stomach or esophagus cancer, breast cancer, ovary cancer, prostate cancer, or liver cancer.

In some embodiments, each somatic variant in the first plurality of somatic variants is a single nucleotide variant or indel mutation.

In some embodiments, the sample of the tumor is a fresh frozen sample.

In some embodiments, each candidate neoantigen in the plurality of candidate neoantigens has a length of N residues, wherein N is 8, 9, 10, 11, 12, 13, or 14. In some embodiments, each candidate neoantigen in the plurality of candidate neoantigens has a length of 9 residues.

In some embodiments, the method further includes excluding from the plurality of candidate neoantigens, or from the final set of candidate neoantigens, those candidate neoantigens that include a mutation from wildtype sequence at the N-terminal residue position.

In some embodiments, the method further includes forming tumor vaccine by solubilizing the final set of neoantigens in a carrier; and administering the vaccine to the human subject. In some embodiments, the forming further comprises including poly-ICLC in the tumor vaccine. In some embodiments, the administering is repeated a plurality of times over a plurality of months.

### 5.7 Example Embodiments for MHC Affinity-Based Neoantigen Selection

Still another aspect of the present disclosure includes a method for identifying a tumor vaccine personalized to a human subject afflicted with a cancer. The method includes (A) determining a first plurality of somatic variants of the subject; and (B) selecting a plurality of candidate neoantigens wherein each neoantigen in at least a subset of candidate neoantigens in the plurality of candidate neoantigens encodes a somatic variant in the plurality of somatic variants. The method further includes (C) determining a respective score for each respective candidate neoantigen in the plurality of candidate neoantigens using a scoring function that comprises: a first scoring term that upweights respective candidate neoantigens having a higher class I major histocompatibility complex (MHC) affinity relative to respective candidate neoantigens having a lower class I MHC affinity, given a class I human leukocyte antigen (HLA) type of the human subject, and a second scoring term that upweights respective candidate neoantigens having a higher class II MHC affinity relative to respective candidate neoantigens having a lower class II MHC affinity, given a class II HLA type of the human subject. The method further includes (D) selecting, for the tumor vaccine, two or more candidate neoantigens in the plurality of candidate neoantigens as a final set of neoantigens based on the respective score of each candidate neoantigen in the plurality of candidate neoantigens.

In some embodiments, the method further includes, after the determining (A), obtaining a first plurality of sequence reads from RNA molecules in a sample of a tumor obtained from the subject; and determining a respective allele-specific expression of each somatic variant in the first plurality of variants using the first plurality of sequence reads, where the scoring function further includes a third scoring term that upweights respective candidate neoantigens representing alleles with higher abundance in the first plurality of sequence reads relative to respective candidate neoantigens representing alleles with lower abundance in the first plurality of sequence reads.

In some embodiments, the method further includes determining, from the first plurality of sequence reads, one or more fusion proteins encoded by the first plurality of sequence reads, where each respective fusion protein in the one or more fusion proteins is a fusion of a portion of a respective first human protein and a portion of a respective second human protein. In some such embodiments, the plurality of candidate neoantigens further comprises a second subset of candidate neoantigens, where each neoantigen in the second subset of candidate neoantigens encodes one or more residues from the portion of the respective first human protein and one or more residues from the portion of the respective second human protein.

In some embodiments, the cancer is glioblastoma or prostate cancer. In some embodiments, the cancer is a carcinoma, a melanoma, a lymphoma/leukemia, a sarcoma, or a neuro-glial tumor. In some embodiments, the cancer is lung cancer, pancreatic cancer, colon cancer, stomach or esophagus cancer, breast cancer, ovary cancer, prostate cancer, or liver cancer.

In some embodiments, each somatic variant in the first plurality of somatic variants is a single nucleotide variant or indel mutation.

In some embodiments, the sample of the tumor is a fresh frozen sample.

In some embodiments, each candidate neoantigen in the plurality of candidate neoantigens has a length of N residues, wherein N is 8, 9, 10, 11, 12, 13, or 14. In some embodiments, each candidate neoantigen in the plurality of candidate neoantigens has a length of 9 residues.

In some embodiments, the method further includes excluding from the plurality of candidate neoantigens, or from the final set of candidate neoantigens, those candidate neoantigens that include a mutation from wildtype sequence at the N-terminal residue position.

In some embodiments, the method further includes forming tumor vaccine by solubilizing the final set of neoantigens in a carrier; and administering the vaccine to the human subject. In some embodiments, the forming further comprises including poly-ICLC in the tumor vaccine. In some embodiments, the administering is repeated a plurality of times over a plurality of months.

EXEMPLIFICATION

### Example 1

### Example 1. Example Pipelines for Prediction of Immunogenic Neoantigens and Administration of Tumor Vaccines Comprising the Same

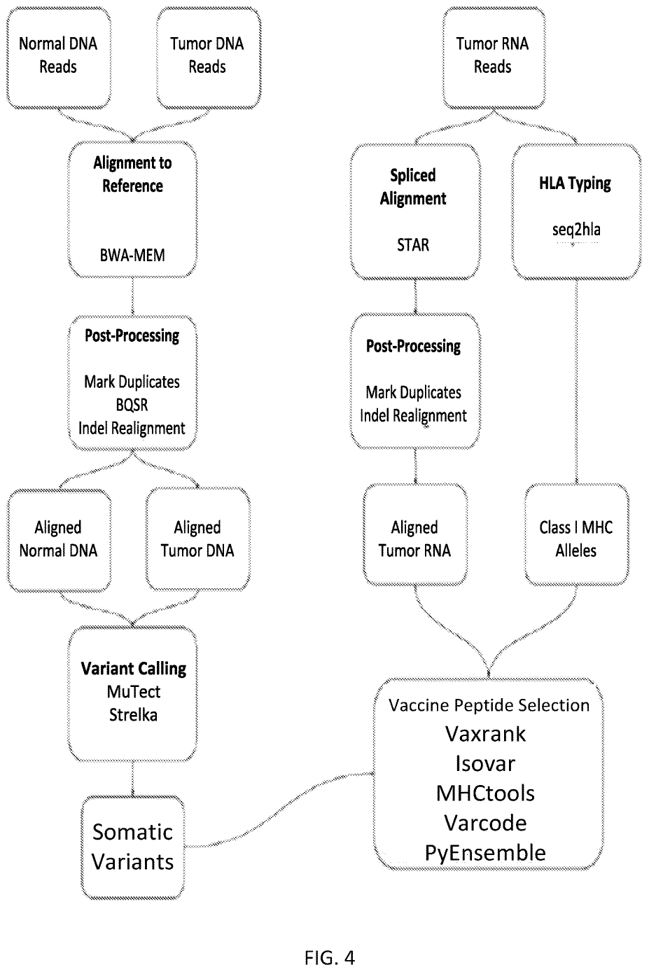

FIG. 4 illustrates an example pipeline for a phase I clinical trial studying the safety and immunogenicity of a multi-peptide personalized genomic vaccine (PGV) for the treatment of cancers. A PGV dose consisted of 10 long synthetic neoantigenic peptides, each containing a somatic variant from the patient's tumor, as well as an immunostimulatory adjuvant poly-ICLC. In the trial, the personalized vaccine was administered in the adjuvant setting, for patients who underwent a complete resection and had no evidence of residual disease.

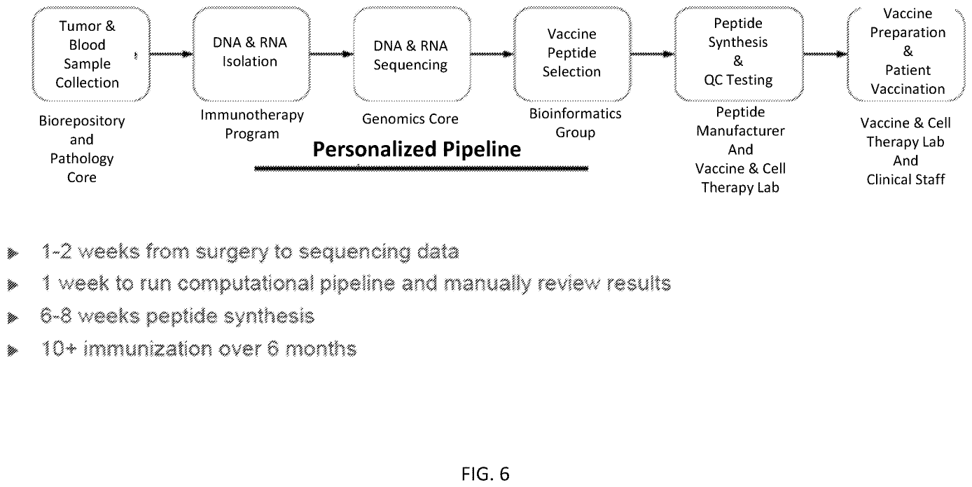

When a new patient enrolled in the trial, their tumor and normal samples were collected and processed to isolate and sequence DNA and RNA. A computational pipeline was then used to select the neoantigenic peptide contents of the vaccine. An overview of an example process from surgery to vaccination is shown in FIG. 6, whereas details of an example computational pipeline are shown in FIG. 4. The candidate vaccine peptides generated by the computational pipeline were ranked by abundance and predicted MHC affinity, which both contribute to immunogenicity. The top 15 ranked candidate peptide sequences were synthesized by a manufacturer and delivered as 10 lyophilized peptides that were purified to sufficient quality and quantity. The peptides were dissolved in DMSO and mixed with poly-ICLC immediately before use. The personalized vaccine was administered as an intracutaneous or subcutaneous injection and was given to the patient 10 times over a span of 6 months. See, e.g., Rubinsteyn et al., “Computational pipeline for the PGV-001 neoantigen vaccine trial,” Front Immunol. 2018; 8; Roudko et al., “Computational prediction and validation of tumor-associated neoantigens,” Front Immunol. 2020; 11:27; Kodysh J, Rubinsteyn A. Openvax: an open-source computational pipeline for cancer neoantigen prediction. Methods Mol Biol. 2020; 2120:147-160; and OpenVax (2018), available on the Internet at openvax.org; each of which is hereby incorporated herein by reference in its entirety.

In particular, as illustrated in FIG. 4, a first example neoantigen tumor vaccine pipeline for a respective subject included the following steps for somatic variant determination: whole exome or genome sequencing (WES or WGS) of tumor and matched normal DNA samples by Illumina short read sequencing platform; quality control of sequencing reads; alignment to the reference genome (e.g., BWA-MEM); post processing (e.g., marking duplicates, base quality score recalibration (BQSR); and/or indel realignment); and comparison of normal and tumor alignments to call somatic variants (e.g., variant calling via tools such as MuTect and/or Strelka), optionally including conversion of coding DNA somatic variants to corresponding mutated peptide sequences, thus generating a plurality of candidate neoantigens.

Moreover, as illustrated in FIG. 4, in some embodiments, the first neoantigen tumor vaccine pipeline further included the following steps: RNA sequencing of tumor RNA, spliced alignment to a reference sequence (e.g., STAR); post processing (e.g., marking duplicates and/or indel realignment); and HLA-allele typing (e.g., seq2hla). In some embodiments, the tumor RNA sequencing included expression analysis of candidate neoantigens, such as phasing co-expressed variants and/or prioritizing expressed variants, in order to validate and/or filter the plurality of candidate neoantigens obtained from DNA.

In some implementations, the first neoantigen tumor vaccine pipeline further included selection of candidate neoantigens, optionally including an assessment of HLA-allele (e.g., class I MHC binding prediction) and mutated epitope (8-11-mer) affinity to call neoantigens. In some implementations, the assessment generated a respective score for each candidate neoantigen, such as a binding score that sums the normalized binding affinities of candidate neoantigens across all alleles of the subject for all lengths between 8-11 residues thereof.

In some implementations, the first neoantigen tumor vaccine pipeline considered various factors that affect somatic variant sensitivity (e.g., sample quality, sequencing library preparation, quantity of sequence reads, sequencing coverage (e.g., 150× for normal sample, 300× for tumor sample, etc.), and/or length of sequence reads (e.g., 125 bp). Moreover, in some implementations, the selection of candidate neoantigens utilized one or more tools known in the art, including but not limited to Vaxrank, Isovar, MHCtools, Varcode, and/or PyEnsembl.

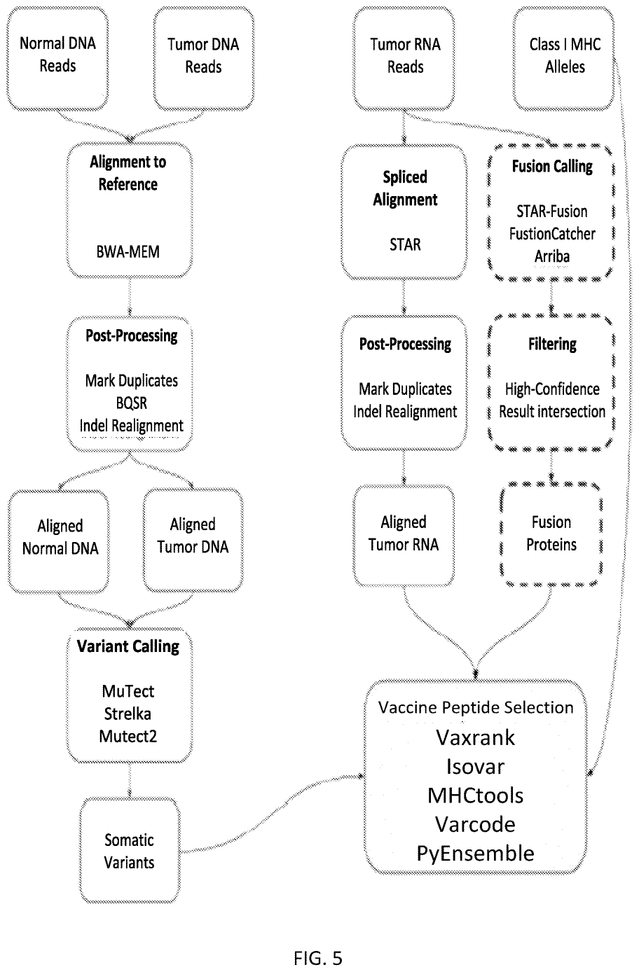

FIG. 5 illustrates a second example neoantigen tumor vaccine pipeline. The second neoantigen tumor vaccine pipeline included the determination of somatic variants as a first subset of candidate neoantigens in a plurality of candidate neoantigens. The second neoantigen tumor vaccine pipeline further included using the RNA sequence reads obtained from tumor RNA to determine one or more fusion proteins encoded by the first plurality of sequence reads, where each respective fusion protein in the one or more fusion proteins was a fusion of a portion of a respective first human protein and a portion of a respective second human protein. In some implementations, such fusion calling utilized tools known in the art, including but not limited to STAR-Fusion, FusionCatcher, Arriba, and/or FusionInspector. In some implementations, the second neoantigen tumor vaccine pipeline further included performing a filtering step to retain high-confidence fusion proteins and/or validate fusion proteins using additional fusion calling approaches (e.g., intersection of multiple tools). The fusion proteins were then optionally converted to corresponding mutated peptide sequences, thus generating a second subset of candidate neoantigens in the plurality of candidate neoantigens.

As illustrated in FIG. 5, in some implementations, the second neoantigen tumor vaccine pipeline further included one or more additional concepts, as described above in the present disclosure. For instance, in some embodiments, the second neoantigen tumor vaccine pipeline included one or more of: determination of neoantigens derived from fusion protein products; avoiding epitopes likely to cross-react with wildtype antigen; preferential vaccination with hydrophobic peptides; consideration of adjacent normal tissue; and/or improved CD4 epitope prediction through incorporation of class II MHC binding prediction.

### Example 2

### Example 2. Prediction of Fusion Proteins for Use as Immunogenic Neoantigens for Prostate Cancer

In an example embodiment, fusion proteins were predicted for use as immunogenic neoantigens for prostate cancer, particularly for use in tumor vaccines.

In certain cancer settings such as prostate cancer, gene fusions are common. For instance, prostate cancer is an outlier with respect to fusions, where a high percentage of prostate cancer cases included the TMPRSS2-ERG gene fusion. See, e.g., Gao et al., “Driver fusions and their implications in the development and treatment of human cancers,” Cell Rep. 2018; 23(1):227-238.e3, which is hereby incorporated herein by reference in its entirety. In contrast, somatic variants such as single nucleotide variants (SNVs) are less common in prostate cancer than in many other solid tumors. Of these, the most frequently mutated genes for SNVs in prostate cancer have been reported to include SPOP, TP53, FOXA1, and/or PTEN. See, e.g., Schumacher and Schreiber, “Neoantigens in cancer immunotherapy,” Science. 2015; 348(6230):69-74; and The Cancer Genome Atlas Research Network, “The molecular taxonomy of primary prostate cancer,” Cell. 2015; 163(4):1011-1025, each of which is hereby incorporated herein by reference in its entirety.

50% of prostate cancers harbor recurrent gene fusions, the most common of which is TMPRSS2-ERG. In addition to ERG, the TMPRSS2 gene can also fuse to other ETS family transcription factors (ETV1, ETV4, etc.). Moreover, 1-2% of prostate cancer tumors have RAF kinase gene fusions, such as SLC45A3-BRAF and/or ESRP1-RAF1. See, e.g., Gao et al., “Driver fusions and their implications in the development and treatment of human cancers,” Cell Rep. 2018; 23(1):227-238.e3; Haffner et al. Androgen-induced TOP2B-mediated double-strand breaks and prostate cancer gene rearrangements. Nat Genet. 2010; 42(8):668-675; and Palanisamy et al. Rearrangements of the RAF kinase pathway in prostate cancer, gastric cancer, and melanoma. Nat Med. 2010; 16(7):793-798, each of which is hereby incorporated herein by reference in its entirety. In some instances, fusion proteins give rise to neoantigens, such that strong predicted MHC binders are located around fusion breakpoints. Thus, in some implementations, neoantigens derived from fusion proteins present an attractive target for identifying and developing tumor vaccines. There are a variety of existing tools to detect gene fusions from sequence reads obtained from RNA molecules in tumor samples (e.g., RNA-seq data), including but not limited to FusionCatcher, STAR-Fusion, Arriba, and/or FusionInspector. In some implementations, the detection of fusion proteins from sequence reads provides reliable fusion detection as well as the prioritization of fusion proteins that can be selected as neoantigens for a tumor vaccine (e.g., within a pipeline for prediction of immunogenic neoantigens and administration of a tumor vaccine comprising the same).

While most existing tumor vaccination pipelines include only small coding mutations such as missense and frameshift mutations, the present example illustrates that tumor vaccines containing a greater number of neoantigens, including both somatic variants and fusion proteins, exhibit improved coverage in patient populations over vaccines that include fewer neoantigens and/or somatic variant neoantigens alone.

In particular, Table 2 illustrates example somatic variants for use in tumor vaccines in combination with one or more fusion proteins that were identified in a population of 497 subjects afflicted with prostate cancer.

TABLE 2
Example Somatic Variants for Shared Antigen Vaccination

| | | | Patients | SEQ | |
|---|---|---|---|---|---|
| Gene | Effect | Patients | (%) | ID No: | Sequence |
| SPOP | p.W131G | 8 | 1.6 | 6 | RAYRFVQGKDGGFKKFIRRDF |
| SPOP | p.F133L | 6 | 1.2 | 7 | YRFVQGKDWGLKKFIRRDFLL |
| SPOP | p.F133V | 6 | 1.2 | 8 | YRFVQGKDWGVKKFIRRDFLL |
| TP53 | p.R248Q | 4 | 0.80 | 9 | CNSSCMGGMNQRPILTIITLE |
| SPOP | p.F102V | 4 | 0.80 | 10 | SCPKSEVRAKVKFSILNAKGE |
| SPOP | p.F133C | 4 | 0.80 | 11 | YRFVQGKDWGCKKFIRRDFLL |
| TP53 | p.G245S | 3 | 0.60 | 12 | NYMCNSSCMGSMNRRPILTII |
| TP53 | p.Y163H | 3 | 0.60 | 13 | PGTRVRAMAIHKQSQHMTEVV |
| PIK3CA | p.E542K | 3 | 0.60 | 14 | KAISTRDPLSKITEQEKDFLW |
| HRAS | p.Q61R | 3 | 0.60 | 15 | CLLDILDTAGREEYSAMRDQY |
| FOXA1 | p.FEN254Y | 3 | 0.60 | 16 | WTLHPDSGNMYGCYLRRQKRFK |

For each gene name shown under the column heading “Gene,” the nature of the somatic variant (e.g., SNV) is shown under the column header “Effect.” The number of subjects in the population (“Patients”), percentage of the population (“Patients (%)”), and amino acid sequence of the neoantigen corresponding to the somatic variant (“Sequence”) are also shown. Mutation calls for somatic variants were obtained from the PanCan Atlas Project (available on the Internet at gdc.cancer.gov/about-data/publications/pancanatlas). 11 somatic coding mutations were found to occur in 3 or more patients. Cumulatively, 45 out of the 497 subjects (9%) in the cohort had at least one of these 11 coding mutations. Given the low percentage of the cohort that exhibited at least one somatic variant, the data show that a tumor vaccine applied to each subject in a subject population (e.g., a “shared antigen vaccine”) developed using single nucleotide somatic mutations alone would be less efficient in prostate cancer. However, it was determined that such somatic variants were likely to be effective if added to a vaccine regimen that included fusion protein-based neoantigens.

FIG. 7 illustrates the improved coverage of the subject population when treated with a shared antigen vaccine that includes both somatic variant neoantigens and fusion protein neoantigens. Compared to the coverage of the subject cohort when treated with the somatic variant-only shared antigen vaccine (9%), the plot in FIG. 7 illustrates a baseline coverage of the subject population of approximately 25% when the shared antigen vaccine includes a TMPRSS2 exon 1-ERG exon 4 fusion protein neoantigen, which increases continually as the shared antigen vaccine is extended to include additional fusion protein neoantigens and SNV neoantigens. Altogether, it is shown that a tumor vaccine that includes 15 vaccine neoantigens, including both SNVs and fusion proteins, achieves 46% coverage of patients in the prostate cancer cohort (HLA not considered). Advantageously, the plot in FIG. 7 further shows the coverage of candidate neoantigens for the tumor vaccine ordered by prevalence (e.g., abundance) from left to right, such that the optimal selection of fusion proteins and SNVs can be performed for inclusion in a tumor vaccine, given a fixed budget of neoantigenic peptides.

Given the above data, the detection of fusion proteins in prostate cancer was further applied to the selection of neoantigens for use in personalized tumor vaccines. Fusion proteins were detected in prostate cancer subjects using a personalized genome vaccine pipeline as described above in Example 1, with reference to FIGS. 5-6. The pipeline detected the fusion protein KANSL1-ARL17A with medium confidence in a tumor sample in a first subject, as well as the fusion protein TMPRSS2-ERG with high confidence in a tumor sample in a second subject. Both of these fusion proteins were further validated as not present in adjacent normal tissue and thus retained as a candidate for immunogenic neoantigens. Thus, the systems and methods disclosed herein were shown to be effective at identifying candidate neoantigens that encode fusion proteins, providing candidates for inclusion in personalized tumor vaccines for prostate cancer subjects enrolled in the trial.

### Example 3

### Example 3. Identification of the EIF3B-FOXK2 Fusion Protein in a Subject Afflicted with Prostate Cancer

The fusion protein EIF3B-FOXK2 was detected in subjects afflicted with prostate cancer, in accordance with an embodiment of the present disclosure. An example implementation of the identification of the EIF3B-FOXK2 fusion protein in a subject afflicted with prostate cancer will now be described.

For fusion calling, various approaches known in the art were employed, including FusionCatcher, STAR-Fusion, Arriba, and FusionInspector. The detection results of each approach were compared. In particular, STAR-Fusion identified 6 total fusion gene pairs (5 validated using FusionInspector). Of these, 2 fusion gene pairs were retained as having a predicted fusion protein sequence with a specific breakpoint. FusionCatcher identified 153 initial fusion gene pairs, of which 46 were determined to have an associated coding sequence with a breakpoint and 19 were retained after removing fusions in known non-cancer dataset. Arriba identified 39 initial fusion gene pairs, of which 12 were high-confidence and 8 were retained as having a predicted fusion protein sequence.

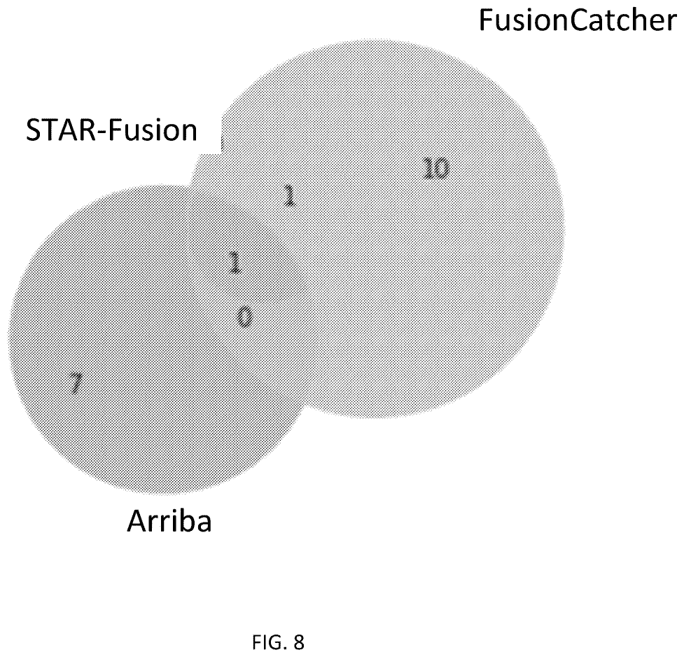

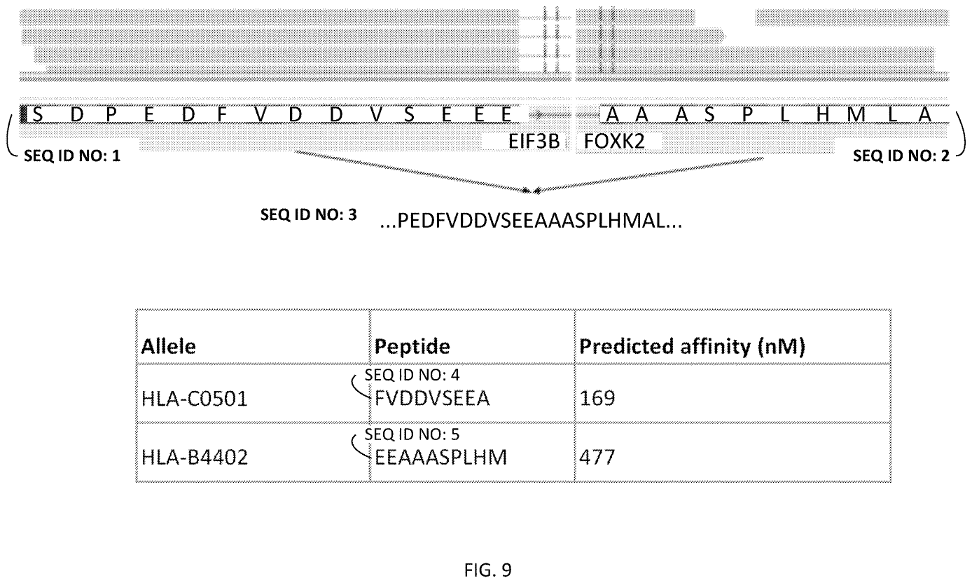

Integration of the results from all three approaches identified a single fusion protein that was common to all approaches, as shown in FIG. 8. Optionally, the method further included confirming any fusion proteins identified in tumor samples against normal samples to confirm that the candidate neoantigen is not also present in normal sample and/or validating any identified fusion proteins using polymerase chain reaction and/or one or more additional sequencing steps (e.g., Sanger sequencing). The shared fusion protein was determined to be EIF3B-FOXK2. EIF3B is an oncogene for many tumor types and has been associated with prostate tumors. See, e.g., Xiang et al. Eukaryotic translation initiation factor 3 subunit b is a novel oncogenic factor in prostate cancer. Mamm Genome. 2020; 31(7-8):197-204, which is hereby incorporated herein by reference in its entirety. A subset of sequence reads in a first plurality of sequence reads obtained from tumor RNA were shown to support the fusion breakpoint for EIF3B-FOXK2. The predicted epitopes for the fusion protein were further determined, based on the class I HLA type of the subject. FIG. 9 shows a schematic of the fusion protein (partial sequence shown as SEQ ID NO: 3: PEDFVDDVSEEAAASPLHMAT) formed from the EIF3B (partial sequence shown as SEQ ID NO: 1: SDPEDFVDDVSEEE) and FOXK2 (partial sequence shown as SEQ ID NO: 2: AAASPLHMLA) proteins, as well as neoantigenic peptide sequences (SEQ ID NO: 4: FVDDVSEEA and SEQ ID NO: 5: EEAAASPLHM) and corresponding class I MHC affinities predicted given a respective class I HLA type (HLA-C0501 and HLA-B4402).

### Example 4

### Example 4. Identification of the TMPRSS2-ERG Fusion Protein in a Subject Afflicted with Prostate Cancer

As described above in Example 2, the fusion protein TMPRSS2-ERG was detected in subjects afflicted with prostate cancer, in accordance with an embodiment of the present disclosure. An example implementation of the identification of the TMPRSS2-ERG fusion protein in a subject afflicted with prostate cancer will now be described.

TMPRSS2 is a membrane-bound serine protease with an androgen response element in its promoter. Testosterone triggers androgen receptor (AR) dimerization, translation to nucleus, and transcription of AR target genes. ERG is a transcription factor from the erythroblast transformation specific (ETS) family. ERG regulates many genes and is strongly implicated in invasion, metastasis, and epigenetic reprogramming. In particular, ERG overexpression and PTEN loss is sufficient for transformation. A common TMPRSS2-ERG rearrangement includes the first intron of TMPRSS2 fused to the third intron of ERG, resulting in an exon 1-exon 4 fusion transcript. This fusion protein is a highly expressed, long-lived protein with ERG transcription factor activity that is expressed by the androgen response element from the TMPRSS2 promoter. The fusion product, however, is missing the ERG N-terminus, which contains the degron for SPOP. The fusion protein is thus resistant to SPOP-mediated degradation. See, e.g., Leung and Sadar, “Non-genomic actions of the androgen receptor in prostate cancer,” Front Endocrinol. 2017; 8; Adamo and Ladomery, “The oncogene ERG: a key factor in prostate cancer,” Oncogene. 2016; 35(4):403-414; Weier et al, “Nucleotide resolution analysis of TMPRSS2 and ERG rearrangements in prostate cancer,” J Pathol. 2013; 230(2):174-183; and An et al., “Truncated ERG oncoproteins from tmprss2-erg fusions are resistant to spop-mediated proteasome degradation,” Mol Cell. 2015; 59(6):904-916, each of which is hereby incorporated herein by reference in its entirety.

A validation procedure was performed for a set of predicted class I MHC binders obtained from Gao et al., using an example bioinformatic approach comprising direct breakpoint detection in RNA-seq. Without being limited to any one theory of operation, in some implementations, the use of direct breakpoint detection in RNA-seq for detection of fusion proteins is predicated on the assumption that precise breakpoint sequences are conserved at the transcript level. For validation, a subset of 160 subjects was obtained from a prostate cancer cohort (TCGA-PRAD), of which 95 subjects were positive for TMPRSS2-ERG fusions (fusion-positive or fusion(+)) and 65 subjects were negative for TMPRSS2-ERG fusions (fusion-negative or fusion(−)).

Briefly, a set of 9 predicted class I MHC binders corresponding to unique neoantigen sequences of the TMPRSS2-ERG fusion protein were obtained for HLA allele contexts HLA-A0201, HLA-A3201, HLA-B1501, HLA-C0303, and HLA-C1402 from Gao et al., “Driver fusions and their implications in the development and treatment of human cancers,” Cell Rep. 2018; 23(1):227-238.e3, which is hereby incorporated herein by reference in its entirety. Using RNA-seq, robust expression of the breakpoint sequences for the set of predicted class I MHC binders was observed in RNA molecules obtained from tumor samples (tumor RNA) of fusion-positive subjects. As a control, DNA molecules from fusion-positive tumor samples and DNA molecules from fusion-positive normal samples did not yield breakpoint sequence expression when assayed using RNA-seq. For further comparison, a set of fusion-negative control samples were also tested, including RNA from fusion-negative tumor samples, DNA from fusion-negative tumor samples, and DNA from fusion-negative normal samples. Breakpoint sequence expression was also not observed in any of the fusion-negative control samples when assayed using RNA-seq.

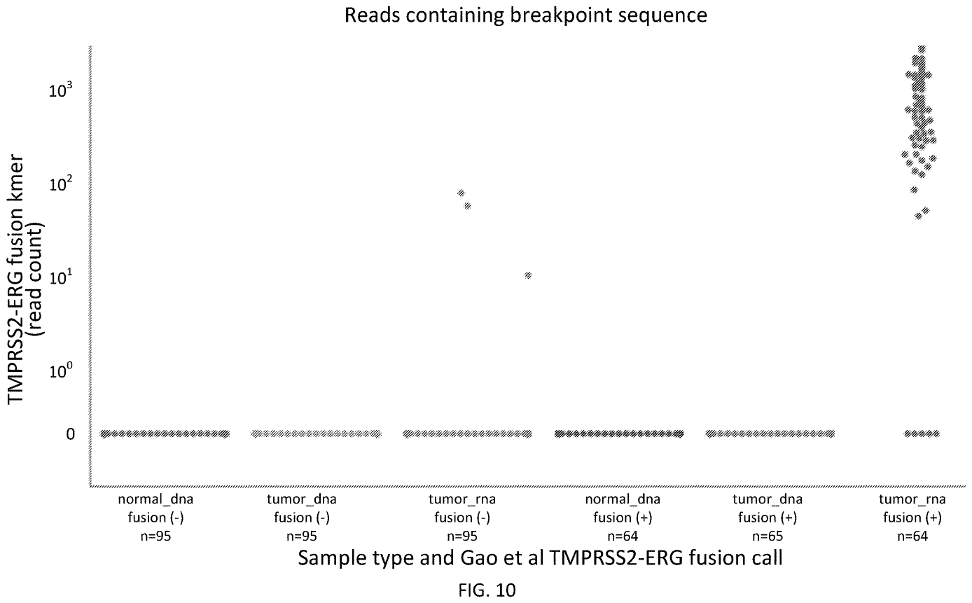

Next, the subset of 160 subjects was analyzed for presence of the most common TMPRSS2-ERG breakpoint sequence. The data points in FIG. 10 indicate the read count of sequence reads containing the target breakpoint sequence for each of the 160 subjects. Almost all subjects with TMPRSS2-ERG fusions identified in Gao et al. included transcripts with the target breakpoint (tumor_rna fusion (+)). Conversely, only 3 fusion-negative subjects exhibited the target breakpoint (tumor_rna fusion (−))

Accordingly, as described above, TMPRSS2-ERG fusions were observed to correspond with conserved transcript sequences. These results show that breakpoint searching for known fusions is likely to identify such fusions at a higher sensitivity than bioinformatics tools designed for de novo identification.

### Example 5

### Example 5. Priming of T Cells from Healthy Donors Against Gene Fusion Proteins Identified for Prostate Cancer

As noted elsewhere herein, in some embodiments, the present disclosure provides example fusion proteins found in cancers, including the TMPRSS2-ERG and EIF3B-FOXK2 gene fusions in prostate cancer.

Peptide sequences spanning gene fusion mutations for the TMPRSS2-ERG and EIF3B-FOXK2 fusions were determined. These included, for EIF3B-FOXK2: SDPEDFVDDVSEEAA (SEQ ID NO: 17), FVDDVSEEAAASPLH (SEQ ID NO: 18), and SEEAAASPLHMLATH (SEQ ID NO: 19); and for TMPRSS2-ERG: MALNSVIPEHRWEGT (SEQ ID NO: 20), RWEGTVQDDQGRLPE (SEQ ID NO: 21), NPVVCTQPKSPSGTV (SEQ ID NO: 22), VCTQPKSPSGTVCTS (SEQ ID NO: 23), and PKSPSGTVCTSRSLI (SEQ ID NO: 24).

T cells from healthy donors (n=6) were obtained and primed in vitro with peptide pools containing either the EIF3B-FOXK2 gene fusion sequences or the TMPRSS2-ERG gene fusion sequences. Positive controls were also performed in which T cells were primed with CEFT, a peptide pool of viral epitopes. Additionally, negative controls were performed in which T cells were primed with DMSO, a vehicle control used to measure any background signal.

Primed T cells were expanded for 8 days prior to re-stimulation with the peptide pools they were primed with. The frequency of antigen-specific T cells was evaluated by measuring effector cytokine production, IFN-γ and TNF-α, by intracellular cytokine staining by flow cytometry for CD4+ and CD8+ T cell subsets. Data was normalized by subtracting the background DMSO stimulation values from each of the test groups (EIF3B-FOXK2, TMPRSS2-ERG, and CEFT).

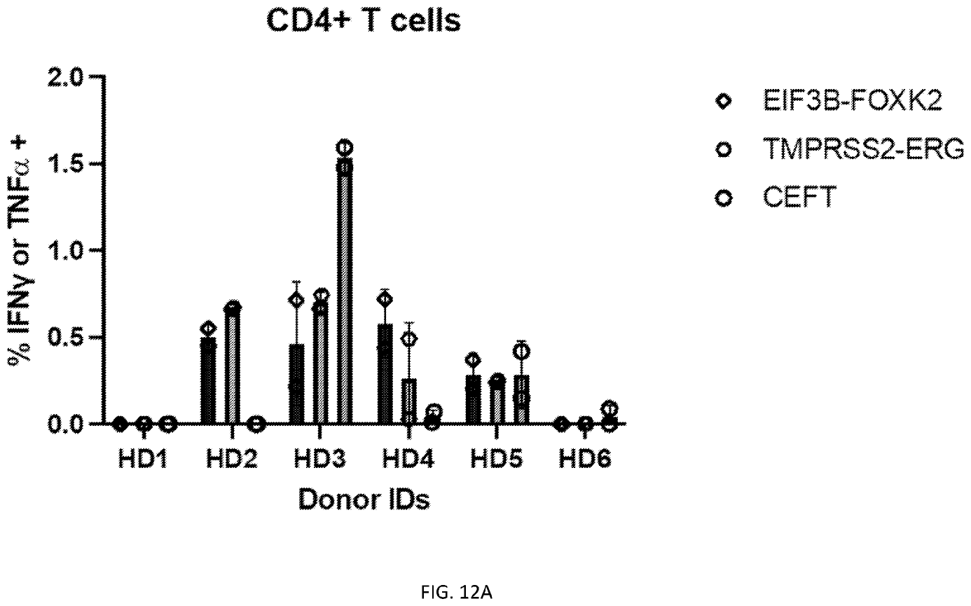

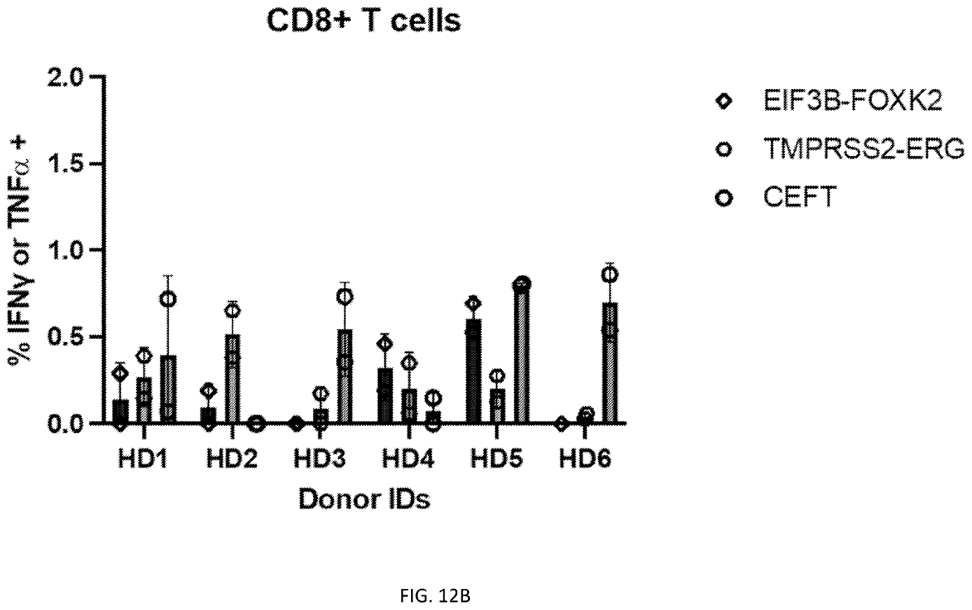

Data is shown in FIGS. 12A-B for each of the healthy donors (HD1-HD6). FIG. 12A illustrates percent IFN-γ and TNF-α in the CD4+ T cell subset after normalizing for background DMSO, for each of the test pools (EIF3B-FOXK2: diamonds; TMPRSS2-ERG: hexagons; and CEFT: circles). FIG. 12B similarly illustrates percent IFN-γ and TNF-α in the CD8+ T cell subset after normalizing for background DMSO, for each of the test pools.

These data illustrate that, in some embodiments, predicted fusion sequences for prostate cancer can be used to generate peptides that can be used to prime naïve T cells from healthy donors, thus inducing an immune response.

### CONCLUSION

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in any combination of FIG. 1 or 2A-B and/or described in FIGS. 3A-F. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method for identifying a tumor vaccine personalized to a human subject afflicted with a cancer, the method comprising:
(A) determining a first plurality of somatic variants of the subject;
(B) obtaining a first plurality of sequence reads from RNA molecules in a sample of a tumor obtained from the subject;
(C) determining, from the first plurality of sequence reads, one or more fusion proteins encoded by the first plurality of sequence reads, wherein each respective fusion protein in the one or more fusion proteins is a fusion of a portion of a respective first human protein and a portion of a respective second human protein;
(D) selecting a plurality of candidate neoantigens comprising a first subset of candidate neoantigens and a second subset of candidate neoantigens, wherein
each neoantigen in the first subset of candidate neoantigens encodes a somatic variant in the first plurality of somatic variants, and
each neoantigen in the second subset of candidate neoantigens encodes one or more residues from the portion of the respective first human protein and one or more residues from the portion of the respective second human protein;
(E) determining a respective score for each respective candidate neoantigen in the plurality of candidate neoantigens using a scoring function that includes a first scoring term that upweights respective candidate neoantigens having a higher class I major histocompatibility complex (MHC) affinity relative to respective candidate neoantigens having a lower class I MHC affinity, given a class I human leukocyte antigen (HLA) type of the human subject; and
(F) selecting, for the tumor vaccine, two or more candidate neoantigens in the plurality of candidate neoantigens as a final set of neoantigens based on the respective score of each candidate neoantigen in the plurality of candidate neoantigens.

2. The method of claim 1, the method further comprising:
determining a respective allele-specific expression of each somatic variant in the first plurality of variants using the first plurality of sequence reads; and
the scoring function further includes a second scoring term that upweights respective candidate neoantigens representing alleles with higher abundance in the first plurality of sequence reads relative to respective candidate neoantigens representing alleles with lower abundance in the first plurality of sequence reads.

3. The method of claim 1, wherein the determining the first plurality of somatic variants of the subject comprises:
obtaining a second plurality of sequence reads from DNA molecules in the tumor sample obtained from the subject;
obtaining a third plurality of sequence reads from DNA molecules in a normal sample obtained from the subject; and
using the second plurality of sequence reads and the third plurality of sequence reads to identify the first plurality of somatic variants of the subject by including in the first plurality of somatic variants those somatic variants observed in the second plurality of sequence reads that are not observed in the third plurality of sequence reads.

4. The method of claim 3, the method further comprising:
performing a first exome sequencing with at least 100× coverage to obtain the second plurality of sequence reads; and
performing a second exome sequencing with at least 100× coverage to obtain the second plurality of sequence reads.

5. The method of claim 3 or 4, wherein the normal sample is a tissue sample proximate to an original location of the tumor in the subject.

6. The method of claim 3 or 4, wherein the normal sample is a blood sample.

7. The method of any one of claims 1-6, wherein the cancer is glioblastoma or prostate cancer.

8. The method of any one of claims 1-7, wherein the cancer is a carcinoma, a melanoma, a lymphoma/leukemia, a sarcoma, or a neuro-glial tumor.

9. The method of any one of claims 1-8, wherein the cancer is lung cancer, pancreatic cancer, colon cancer, stomach or esophagus cancer, breast cancer, ovary cancer, prostate cancer, or liver cancer.

10. The method of any one of claims 1-9, wherein each somatic variant in the first plurality of somatic variants is a single nucleotide variant or indel mutation.

11. The method of any one of claims 1-10, wherein the sample of the tumor is a fresh frozen sample.

12. The method of any one of claims 1-11, wherein each respective candidate neoantigen in the plurality of candidate neoantigens has a length of N residues, wherein N is 8, 9, 10, 11, 12, 13, or 14.

13. The method of any one of claims 1-11, wherein each respective candidate neoantigen in the plurality of candidate neoantigens has a length of 9 residues.

14. The method of any one of claims 1-13, the method further comprising excluding from the plurality of candidate neoantigens, or from the final set of candidate neoantigens, those candidate neoantigens that include a mutation from wildtype sequence at the N-terminal residue position.

15. The method of any one of claims 1-14, the method further comprising determining the class I HLA type of the human subject using the first plurality of sequencing reads.

16. The method of any one of claims 1-14, the method further comprising determining the class I HLA type of the human subject using a polymerase chain reaction using a biological sample from the cancer subject.

17. The method of any one of claims 1-16, the method further comprising:
determining a hydrophobicity of each respective candidate neoantigen in the plurality of candidate neoantigens; and
the scoring function further includes a scoring term for hydrophobicity of the respective candidate neoantigen.

18. The method of claim 17, wherein the determining the respective hydrophobicity of each candidate neoantigen in the plurality of candidate neoantigens comprises assigning the respective candidate neoantigen the maximum hydrophobicity score of any 7-mer within the respective candidate neoantigen.

19. The method of claim 17 or 18, wherein the scoring term for hydrophobicity of the respective candidate neoantigen upweights more hydrophobic candidate neoantigens relative to less hydrophobic candidate neoantigens.

20. The method of any one of claims 1-19, wherein the scoring function further includes a term for class II MHC affinity of the respective candidate neoantigen, given a class II HLA type of the human subject, that upweights respective candidate neoantigens having higher class II MHC affinity than respective candidate neoantigens having lower class II MHC affinity.

21. The method of any one of claims 1-20, the method further comprising:
using the first plurality of sequence reads to validate a plurality of indel mutations present in the tumor sample; and wherein
the plurality of candidate neoantigens comprises a third subset of candidate neoantigens, and
each neoantigen in the third subset of the plurality of candidate neoantigens encodes all or a portion of an indel mutation in the plurality of indel mutations.

22. The method of any one of claims 1-21, wherein the first plurality of sequence reads comprises 1×10⁶ sequence reads.

23. The method of any one of claims 1-22, wherein the plurality of candidate neoantigens comprises 5 candidate neoantigens.

24. The method of any one of claims 1-23, wherein the final set of neoantigens consists of between two and twenty neoantigens in the plurality of candidate neoantigens.

25. The method of any one of claims 1-24, wherein the final set of neoantigens consists of one or more neoantigens from the first subset of the plurality of candidate neoantigens and one or more neoantigens from the second subset of the plurality of candidate neoantigens.

26. The method of claim 21, wherein the final set of neoantigens consists of one or more neoantigens from the first subset of the plurality of candidate neoantigens, one or more neoantigens from the second subset of the plurality of candidate neoantigens, and one or more neoantigens from the third subset of the plurality of candidate neoantigens.

27. The method of any one of claims 1-26, the method further comprising:
forming tumor vaccine by solubilizing the final set of neoantigens in a carrier; and
administering the vaccine to the human subject.

28. The method of claim 27, wherein the forming further comprises including poly-ICLC in the tumor vaccine.

29. The method of claim 27 or 28, wherein the administering is repeated a plurality of times over a plurality of months.

30. A system for identifying a tumor vaccine personalized to a human subject afflicted with a cancer, comprising:
a memory;
one or more processors; and
one or more modules stored in the memory and configured for execution by the one or more processors, the one or more modules comprising instructions for:
(A) determining a first plurality of somatic variants of the subject;
(B) obtaining, in electronic form, a first plurality of sequence reads from RNA molecules in a sample of a tumor obtained from the subject;
(C) determining, from the first plurality of sequence reads, one or more fusion proteins encoded by the first plurality of sequence reads, wherein each respective fusion protein in the one or more fusion proteins is a fusion of a portion of a respective first human protein and a portion of a respective second human protein;
(D) selecting a plurality of candidate neoantigens comprising a first subset of candidate neoantigens and a second subset of candidate neoantigens, wherein
each neoantigen in the first subset of candidate neoantigens encodes a somatic variant in the first plurality of somatic variants, and
each neoantigen in the second subset of candidate neoantigens encodes one or more residues from the portion of the respective first human protein and one or more residues from the portion of the respective second human protein;
(E) determining a respective score for each respective candidate neoantigen in the plurality of candidate neoantigens using a scoring function that includes a first scoring term that upweights respective candidate neoantigens having a higher class I major histocompatibility complex (MHC) affinity relative to respective candidate neoantigens having a lower class I MHC affinity, given a class I human leukocyte antigen (HLA) type of the human subject; and
(F) selecting, for the tumor vaccine, two or more candidate neoantigens in the plurality of candidate neoantigens as a final set of neoantigens based on the respective score of each candidate neoantigen in the plurality of candidate neoantigens.

31. A non-transitory computer readable storage medium for identifying a tumor vaccine personalized to a human subject afflicted with a cancer, the non-transitory computer readable storage medium storing one or more programs for execution by one or more processors of a computer system, the one or more computer programs comprising instructions for:
(A) determining a first plurality of somatic variants of the subject;
(B) obtaining, in electronic form, a first plurality of sequence reads from RNA molecules in a sample of a tumor obtained from the subject;
(C) determining, from the first plurality of sequence reads, one or more fusion proteins encoded by the first plurality of sequence reads, wherein each respective fusion protein in the one or more fusion proteins is a fusion of a portion of a respective first human protein and a portion of a respective second human protein;
(D) selecting a plurality of candidate neoantigens comprising a first subset of candidate neoantigens and a second subset of candidate neoantigens, wherein
each neoantigen in the first subset of candidate neoantigens encodes a somatic variant in the first plurality of somatic variants, and
each neoantigen in the second subset of candidate neoantigens encodes one or more residues from the portion of the respective first human protein and one or more residues from the portion of the respective second human protein;
(E) determining a respective score for each respective candidate neoantigen in the plurality of candidate neoantigens using a scoring function that includes a first scoring term that upweights respective candidate neoantigens having a higher class I major histocompatibility complex (MHC) affinity relative to respective candidate neoantigens having a lower class I MHC affinity, given a class I human leukocyte antigen (HLA) type of the human subject; and
(F) selecting, for the tumor vaccine, two or more candidate neoantigens in the plurality of candidate neoantigens as a final set of neoantigens based on the respective score of each candidate neoantigen in the plurality of candidate neoantigens.

32. A tumor vaccine comprising a plurality of neoantigenic peptides and an adjuvant personalized for a subject afflicted with a cancer, wherein
a first neoantigen in the tumor vaccine encodes a single nucleotide polymorphism present in RNA molecules in a tumor biopsy obtained from the subject, and
a second neoantigen in the tumor vaccine encodes one or more residues from a portion of a first human protein and one or more residues from a portion of a second human protein, wherein the tumor biopsy includes RNA molecules that are a fusion of the first human protein and the second human protein.

33. The tumor vaccine of claim 32, wherein the cancer is prostate cancer and the second neoantigen encodes a fusion represented in Table 1.

34. The tumor vaccine of claim 32 or 33, wherein the cancer is prostate cancer and wherein the single nucleotide polymorphism is in SPOP, TP53, FOXA1 or PTEN.

35. The tumor vaccine of any one of claims 32-34, wherein the cancer is a carcinoma, a melanoma, a lymphoma/leukemia, a sarcoma, or a neuro-glial tumor.

36. The tumor vaccine of any one of claims 32-35, wherein the cancer is lung cancer, pancreatic cancer, colon cancer, stomach or esophagus cancer, breast cancer, ovary cancer, prostate cancer, or liver cancer.

37. The tumor vaccine of any one of claims 32-36, wherein the tumor biopsy is a fresh frozen sample.

38. The tumor vaccine of any one of claims 32-37, wherein each respective neoantigenic peptide in the plurality of neoantigenic peptides has a length of N residues, wherein N is 8, 9, 10, 11, 12, 13, or 14.

39. The tumor vaccine of any one of claims 32-38, wherein each respective neoantigenic peptide in the plurality of neoantigenic peptides has a length of 9 residues.

40. The tumor vaccine of any one of claims 32-39, wherein each neoantigenic peptide in the plurality of neoantigenic peptides does not include a mutation from wildtype sequence at the N-terminal residue position.

41. The tumor vaccine of any one of claims 32-40, wherein the plurality of neoantigenic peptides is solubilized in a carrier.

42. The tumor vaccine of any one of claims 32-41, wherein the adjuvant is poly-ICLC.

43. A method, comprising administering the tumor vaccine of any one of claims 32-42 to the subject.

44. The method of claim 43, wherein the administering is repeated a plurality of times over a plurality of months.

45. A method for identifying a tumor vaccine personalized to a human subject afflicted with a cancer, the method comprising:
(A) determining a first plurality of somatic variants of the subject;
(B) selecting a plurality of candidate neoantigens wherein each neoantigen in at least a subset of candidate neoantigens in the plurality of candidate neoantigens encodes a somatic variant in the first plurality of somatic variants;
(C) determining a respective score for each respective candidate neoantigen in the plurality of candidate neoantigens using a scoring function that comprises:
a first scoring term that upweights candidate neoantigens having a higher class I major histocompatibility complex (MHC) affinity relative to candidate neoantigens having a lower class I MHC affinity, given a class I human leukocyte antigen (HLA) type of the human subject, and
a second scoring term that upweights candidate neoantigens having higher hydrophobicity relative to candidate neoantigens having lower hydrophobicity; and
(D) selecting, for the tumor vaccine, two or more candidate neoantigens in the plurality of candidate neoantigens as a final set of neoantigens based on the respective score of each candidate neoantigen in the plurality of candidate neoantigens.

46. The method of claim 45, further comprising, after the determining (A):
obtaining a first plurality of sequence reads from RNA molecules in a sample of a tumor obtained from the subject; and
determining a respective allele-specific expression of each somatic variant in the first plurality of variants using the first plurality of sequence reads, wherein
the scoring function further includes a third scoring term that upweights respective candidate neoantigens representing alleles with higher abundance in the first plurality of sequence reads relative to respective candidate neoantigens representing alleles with lower abundance in the first plurality of sequence reads.

47. The method of claim 46, further comprising:
determining, from the first plurality of sequence reads, one or more fusion proteins encoded by the first plurality of sequence reads, wherein each respective fusion protein in the one or more fusion proteins is a fusion of a portion of a respective first human protein and a portion of a respective second human protein, wherein:
the plurality of candidate neoantigens further comprises a second subset of candidate neoantigens, wherein each neoantigen in the second subset of candidate neoantigens encodes one or more residues from the portion of the respective first human protein and one or more residues from the portion of the respective second human protein.

48. The method of any one of claims 45-47, wherein the cancer is glioblastoma or prostate cancer.

49. The method of any one of claims 45-48, wherein the cancer is a carcinoma, a melanoma, a lymphoma/leukemia, a sarcoma, or a neuro-glial tumor.

50. The method of any one of claims 45-49, wherein the cancer is lung cancer, pancreatic cancer, colon cancer, stomach or esophagus cancer, breast cancer, ovary cancer, prostate cancer, or liver cancer.

51. The method of any one of claims 45-50, wherein each somatic variant in the first plurality of somatic variants is a single nucleotide variant or indel mutation.

52. The method of claim 46 or 47, wherein the tumor biopsy is a fresh frozen sample.

53. The method of any one of claims 45-52, wherein each candidate neoantigen in the plurality of candidate neoantigens has a length of N residues, wherein N is 8, 9, 10, 11, 12, 13, or 14.

54. The method of any one of claims 45-53, wherein each candidate neoantigen in the plurality of candidate neoantigens has a length of 9 residues.

55. The method of any one of claims 45-54, the method further comprising excluding from the plurality of candidate neoantigens, or from the final set of candidate neoantigens, those candidate neoantigens that include a mutation from wildtype sequence at the N-terminal residue position.

56. The method of any one of claims 45-55, the method further comprising:
forming tumor vaccine by solubilizing the final set of neoantigens in a carrier; and
administering the vaccine to the human subject.

57. The method of claim 56, wherein the forming further comprises including poly-ICLC in the tumor vaccine.

58. The method of claim 56 or 57, wherein the administering is repeated a plurality of times over a plurality of months.

59. A method for identifying a tumor vaccine personalized to a human subject afflicted with a cancer, the method comprising:
(A) obtaining a first plurality of sequence reads from RNA molecules in a sample of a tumor obtained from the subject;
(B) obtaining a second plurality of sequence reads from DNA molecules in the tumor sample obtained from the subject;
(C) obtaining a third plurality of sequence reads from DNA molecules in a normal tissue sample proximate to an original location of the tumor in the subject; and
(D) using the second plurality of sequence reads and the third plurality of sequence reads to identify a plurality of somatic variants of the subject by including in the plurality of somatic variants those somatic variants observed in the second plurality of sequence reads that are not observed in the third plurality of sequence reads;
(E) selecting a plurality of candidate neoantigens wherein each neoantigen in at least a subset of candidate neoantigens in the plurality of candidate neoantigens encodes a somatic variant in the plurality of somatic variants;
(F) determining a respective score for each respective candidate neoantigen in the plurality of candidate neoantigens using a scoring function that comprises a first scoring term that upweights candidate neoantigens having a higher class I major histocompatibility complex (MHC) affinity relative to candidate neoantigens having a lower class I MHC affinity, given a class I human leukocyte antigen (HLA) type of the human subject; and
(G) selecting, for the tumor vaccine, two or more candidate neoantigens in the plurality of candidate neoantigens as a final set of neoantigens based on the respective score of each candidate neoantigen in the plurality of candidate neoantigens.

60. The method of claim 59, further comprising:
determining, from the first plurality of sequence reads, one or more fusion proteins encoded by the first plurality of sequence reads, wherein each respective fusion protein in the one or more fusion proteins is a fusion of a portion of a respective first human protein and a portion of a respective second human protein, wherein:
the plurality of candidate neoantigens further comprises a second subset of candidate neoantigens, wherein each neoantigen in the second subset of candidate neoantigens encodes one or more residues from the portion of the respective first human protein and one or more residues from the portion of the respective second human protein.

61. The method of claim 59 or 60, further comprising:
determining a respective allele-specific expression of each somatic variant in the first plurality of variants using the first plurality of sequence reads, wherein
the scoring function further includes a second scoring term that upweights respective candidate neoantigens representing alleles with higher abundance in the first plurality of sequence reads relative to respective candidate neoantigens representing alleles with lower abundance in the first plurality of sequence reads.

62. The method of any one of claims 59-61, wherein the cancer is glioblastoma or prostate cancer.

63. The method of any one of claims 59-62, wherein the cancer is a carcinoma, a melanoma, a lymphoma/leukemia, a sarcoma, or a neuro-glial tumor.

64. The method of any one of claims 59-63, wherein the cancer is lung cancer, pancreatic cancer, colon cancer, stomach or esophagus cancer, breast cancer, ovary cancer, prostate cancer, or liver cancer.

65. The method of any one of claims 59-64, wherein each somatic variant in the first plurality of somatic variants is a single nucleotide variant or indel mutation.

66. The method of any one of claims 59-65, wherein the sample of the tumor is a fresh frozen sample.

67. The method of any one of claims 59-66, wherein each candidate neoantigen in the plurality of candidate neoantigens has a length of N residues, wherein N is 8, 9, 10, 11, 12, 13, or 14.

68. The method of any one of claims 59-67, wherein each candidate neoantigen in the plurality of candidate neoantigens has a length of 9 residues.

69. The method of any one of claims 59-68, the method further comprising excluding from the plurality of candidate neoantigens, or from the final set of candidate neoantigens, those candidate neoantigens that include a mutation from wildtype sequence at the N-terminal residue position.

70. The method of any one of claims 59-69, the method further comprising:
forming tumor vaccine by solubilizing the final set of neoantigens in a carrier; and
administering the vaccine to the human subject.

71. The method of claim 70, wherein the forming further comprises including poly-ICLC in the tumor vaccine.

72. The method of claim 70 or 71, wherein the administering is repeated a plurality of times over a plurality of months.

73. A method for identifying a tumor vaccine personalized to a human subject afflicted with a cancer, the method comprising:
(A) determining a first plurality of somatic variants of the subject;
(B) selecting a plurality of candidate neoantigens wherein each neoantigen in at least a subset of candidate neoantigens in the plurality of candidate neoantigens encodes a somatic variant in the plurality of somatic variants;
(C) determining a respective score for each respective candidate neoantigen in the plurality of candidate neoantigens using a scoring function that comprises:
a first scoring term that upweights respective candidate neoantigens having a higher class I major histocompatibility complex (MHC) affinity relative to respective candidate neoantigens having a lower class I MHC affinity, given a class I human leukocyte antigen (HLA) type of the human subject, and
a second scoring term that upweights respective candidate neoantigens having a higher class II MHC affinity relative to respective candidate neoantigens having a lower class II MHC affinity, given a class II HLA type of the human subject; and
(D) selecting, for the tumor vaccine, two or more candidate neoantigens in the plurality of candidate neoantigens as a final set of neoantigens based on the respective score of each candidate neoantigen in the plurality of candidate neoantigens.

74. The method of claim 73, further comprising, after the determining (A):
obtaining a first plurality of sequence reads from RNA molecules in a sample of a tumor obtained from the subject; and
determining a respective allele-specific expression of each somatic variant in the first plurality of variants using the first plurality of sequence reads, wherein
the scoring function further includes a third scoring term that upweights respective candidate neoantigens representing alleles with higher abundance in the first plurality of sequence reads relative to respective candidate neoantigens representing alleles with lower abundance in the first plurality of sequence reads.

75. The method of claim 74, further comprising:
determining, from the first plurality of sequence reads, one or more fusion proteins encoded by the first plurality of sequence reads, wherein each respective fusion protein in the one or more fusion proteins is a fusion of a portion of a respective first human protein and a portion of a respective second human protein, wherein:
the plurality of candidate neoantigens further comprises a second subset of candidate neoantigens, wherein each neoantigen in the second subset of candidate neoantigens encodes one or more residues from the portion of the respective first human protein and one or more residues from the portion of the respective second human protein.

76. The method of any one of claims 73-75, wherein the cancer is glioblastoma or prostate cancer.

77. The method of any one of claims 73-76, wherein the cancer is a carcinoma, a melanoma, a lymphoma/leukemia, a sarcoma, or a neuro-glial tumor.

78. The method of any one of claims 73-77, wherein the cancer is lung cancer, pancreatic cancer, colon cancer, stomach or esophagus cancer, breast cancer, ovary cancer, prostate cancer, or liver cancer.

79. The method of any one of claims 73-78, wherein each somatic variant in the first plurality of somatic variants is a single nucleotide variant or indel mutation.

80. The method of claim 74 or 75, wherein the sample of the tumor is a fresh frozen sample.

81. The method of any one of claims 73-80, wherein each candidate neoantigen in the plurality of candidate neoantigens has a length of N residues, wherein N is 8, 9, 10, 11, 12, 13, or 14.

82. The method of any one of claims 73-81, wherein each candidate neoantigen in the plurality of candidate neoantigens has a length of 9 residues.

83. The method of any one of claims 73-82, the method further comprising excluding from the plurality of candidate neoantigens, or from the final set of candidate neoantigens, those candidate neoantigens that include a mutation from wildtype sequence at the N-terminal residue position.

84. The method of any one of claims 73-83, the method further comprising:
forming tumor vaccine by solubilizing the final set of neoantigens in a carrier; and
administering the vaccine to the human subject.

85. The method of claim 84, wherein the forming further comprises including poly-ICLC in the tumor vaccine.

86. The method of claim 84 or 85, wherein the administering is repeated a plurality of times over a plurality of months.


SEQUENCE LISTING (24 sequences)

SEQ ID NO: 1 - protein, 14 aa, synthetic construct
SDPEDFVDDVSEEE

SEQ ID NO: 2 - protein, 10 aa, Synthetic construct
AAASPLHMLA

SEQ ID NO: 3 - protein, 21 aa, synthetic construct
PEDFVDDVSEEAAASPLHMAT

SEQ ID NO: 4 - protein, 9 aa, synthetic construct
FVDDVSEEA

SEQ ID NO: 5 - protein, 10 aa, synthetic construct
EEAAASPLHM

SEQ ID NO: 6 - protein, 21 aa, synthetic construct
RAYRFVQGKDGGFKKFIRRDF

SEQ ID NO: 7 - protein, 21 aa, synthetic construct
YRFVQGKDWGLKKFIRRDFLL

SEQ ID NO: 8 - protein, 21 aa, synthetic construct
YRFVQGKDWGVKKFIRRDFLL

SEQ ID NO: 9 - protein, 21 aa, synthetic construct
CNSSCMGGMNQRPILTIITLE

SEQ ID NO: 10 - protein, 21 aa, synthetic construct
SCPKSEVRAKVKFSILNAKGE

SEQ ID NO: 11 - protein, 21 aa, synthetic construct
YRFVQGKDWGCKKFIRRDFLL

SEQ ID NO: 12 - protein, 21 aa, synthetic construct
NYMCNSSCMGSMNRRPILTII

SEQ ID NO: 13 - protein, 21 aa, synthetic construct
PGTRVRAMAIHKQSQHMTEVV

SEQ ID NO: 14 - protein, 21 aa, synthetic construct
KAISTRDPLSKITEQEKDFLW

SEQ ID NO: 15 - protein, 21 aa, synthetic construct
CLLDILDTAGREEYSAMRDQY

SEQ ID NO: 16 - protein, 22 aa, synthetic construct
WTLHPDSGNMYGCYLRRQKRFK

SEQ ID NO: 17 - protein, 15 aa, synthetic construct
SDPEDFVDDVSEEAA

SEQ ID NO: 18 - protein, 15 aa, synthetic construct
SEEAAASPLHMLATH

SEQ ID NO: 19 - protein, 15 aa, synthetic construct
FVDDVSEEAAASPLH

SEQ ID NO: 20 - protein, 24 aa, synthetic construct
TMPRSSERGMALNSVIPEHRWEGT

SEQ ID NO: 21 - protein, 15 aa, synthetic construct
RWEGTVQDDQGRLPE

SEQ ID NO: 22 - protein, 15 aa, synthetic construct
NPVVCTQPKSPSGTV

SEQ ID NO: 23 - protein, 15 aa, synthetic construct
VCTQPKSPSGTVCTS

SEQ ID NO: 24 - protein, 15 aa, synthetic construct
PKSPSGTVCTSRSLI